(19) United States
(12) Patent Application Publication
(10) Pub. No.: US 2025/0368979 A1
(43) Pub. Date: Dec. 4, 2025

(54) BEADS COMPRISING CELLULOSE FOR DNA EXTRACTION AND NORMALIZATION

(71) Applicant: ILLUMINA, INC., San Diego, CA (US)

(72) Inventors: Jian-Sen Li, San Diego, CA (US); Xin Sheng, San Diego, CA (US); Yuan Ding, San Diego, CA (US); Huei-Yu Wang Ko, San Francisco, CA (US); Jessica Cifelli, San Diego, CA (US); Johnathan Parker, Wheatley (GB); Chia-Ling Hsieh, San Diego, CA (US); Anupama Khanna, Madison, WI (US)

(21) Appl. No.: 18/722,105

(22) PCT Filed: Dec. 19, 2022
(86) PCT No.: PCT/US2022/053379

§ 371 Date: Jun. 20, 2024

Related U.S. Application Data
(60) Provisional application No. 63/292,174, filed on Dec. 21, 2021.
(60) Provisional application No. 63/328,569, filed on Apr. 7, 2022.

(51) Int. Cl. C12N 15/10

(52) U.S. Cl. CPC C12N 15/1006; C12N 15/1093

(57) ABSTRACT

Described herein are methods of treating beads comprising cellulose. In some embodiments, the treatment may be with a relatively low concentration of NaOH. The treated beads may be used in methods of nucleic acid extraction from a sample and/or normalization of a library. Also described herein are untreated beads comprising cellulose for library normalization. Normalization with beads comprising cellulose may yield libraries with greater GC bias.

### CROSS-REFERENCE TO RELATED APPLICATION

This application is a 371 national stage application of PCT/2022/053379, filed Dec. 19, 2022, which claims the benefit of priority of U.S. Provisional Application No. 63/328,569, filed Apr. 7, 2022, and the benefit of U.S. Provisional Application No. 63/292,174, filed Dec. 21, 2021, the contents of which are each incorporated by reference herein in their entireties for any purpose.

### DESCRIPTION

### Field

Described herein are methods of treating beads comprising cellulose. The treatment may be with a relatively low concentration of NaOH. These treated beads may be used in methods of nucleic acid extraction from a sample and/or normalization of a library. Also described herein are untreated beads comprising cellulose for library normalization.

### Background

Circulating cell free DNA (cfDNA) comprises degraded DNA fragments released into the blood plasma. cfDNA can be used to describe various forms of DNA freely circulating the bloodstream, including circulating tumor DNA (ctDNA) and cell-free fetal DNA (cffDNA). cfDNA has been shown to be a useful biomarker for a range of diagnostics, including those for cancer and fetal medicine. The use of cfDNA diagnostics includes but is not limited to trauma, sepsis, aseptic inflammation, myocardial infarction, stroke, transplantation, diabetes, and sickle cell disease. Generating sequencing data from ctDNA or other cfDNA requires a number of different processes. For example, a protocol for sequencing ctDNA may include extraction of ctDNA from plasma, normalization of extracted ctDNA, and library preparation before sequencing. Further, library preparation before sequencing may require purifying ctDNA (i.e., separating the library from reagents used for library preparation), size selection to enrich for appropriately sized fragments, and normalization.

cfDNA comprises double-stranded DNA fragments that are generally short (less than 200 base pairs (bp)) and normally at a low concentration in the blood or plasma (10-100 ng/mL). In healthy non-pregnant individuals, plasma cfDNA is believed to be derived primarily from apoptosis of normal cells of the hematopoietic lineage, with minimal contributions from other tissues.

The small DNA fragments comprised in cfDNA provide useful genetic information, which can potentially be unlocked quickly and accurately with next generation sequencing technologies. For example, non-invasive prenatal testing (NIPT) uses cfDNA derived from a pregnant woman to evaluate possible chromosomal conditions in the fetus. NIPT can be used as a prenatal screening test that can be performed as early as 10 weeks of pregnancy using a single blood draw. Presently, NIPT kits often use filter-based cfDNA extraction (see, for example, purification of cfDNA from plasma by binding onto a binding plate as described in VeriSeq NIPT Solution v2 Package Insert, Illumina, Document #1000000078751v06, August 2021). However, bead-based extraction of cfDNA could provide benefits, such as allowing greater ease of use and potential for automation of assays with magnetic beads.

Liquid biopsies are another diagnostic that uses cfDNA. Liquid biopsies are noninvasive tests that detect fragments of DNA or cells in blood or, occasionally, other bodily fluids. cfDNA derived from apoptotic and necrotic cells may be present in the bodily fluid. For example, liquid biopsies may be used to measure circulating tumor DNA (ctDNA) or other cfDNA in the blood of a patient (see, for example, Maltoni et al., Oncotarget 8 (10): 16642-16649 (2017)).

An efficient and simple way to isolate cfDNA without genomic DNA or other nucleic acid contamination is needed. Generally, achieving a desired yield of cfDNA extraction with commercial beads is difficult because suitable beads for cfDNA extraction must both have high binding and easy elution of the cfDNA. For example, a particular bead may tightly bind cfDNA, but a user may have difficulty in eluting the cfDNA off the bead without damaging the cfDNA (such as generating smaller fragments that are undesired during elution). Thus, many beads that can tightly bind cfDNA are not suitable for cfDNA extraction, as these beads do not allow for elution of the cfDNA under gentle enough conditions to maintain cfDNA integrity and fragment size.

Described herein are treated beads comprising cellulose, such as cellulose-coated beads, that may be magnetic. Treated cellulose-coated magnetic beads described herein have use in isolating cfDNA for NIPT, liquid biopsy, and other methodologies that use cfDNA. In embodiments with magnetic beads, the present beads also have advantages due to the compatibility of magnetic beads with automation.

The present treated cellulose-coated beads may also be used for improved methods of normalization of libraries before sequencing. The size of the reaction and the small number of nucleic acids in many libraries often make it difficult to capture and normalize. For example, a user may need to normalize DNA down to a very small amount of less than 100 pg/μL of material. Most bead-based solutions for capture and normalization have far too many beads and require large amounts of DNA to be successful in such situations. Additionally, a portion of DNA is frequently denatured during capture and release for present methods of manual normalization, which may negatively influence downstream methodologies.

Many DNA-binding beads, such streptavidin beads or carboxylate (solid phase reversible immobilization, SPRI) beads, have a binding capacity for DNA that is too high to be used for library normalization. For example, such low concentrations of streptavidin or carboxylate beads would be necessary such that normalization is difficult to perform and may require bead dilution steps. Some protocols with streptavidin beads may also require that “dummy beads” that do not bind DNA be included in normalization protocols for acceptable results. Further, binding of DNA to streptavidin beads and certain other DNA-binding beads may be so strong (such as with a covalent bond) that denaturation is required for elution of the DNA from the bead, and this elution can cause DNA damage.

In summary, normalization of high-molecular weight DNA in a small sample volume has been difficult and may require manual normalization steps. However, manual normalization can lead to time delays and user-intensive methods requiring calculations. Cellulose-coated magnetic beads, as described herein, can improve and simplify library normalization workflows. As described herein, cellulose-coated beads, either untreated or treated with low concentrations of NaOH, can improve library normalization by avoiding degradation of the library, allowing easier processing, and improving results for libraries with low DNA input levels.

### SUMMARY

In accordance with the description, described herein are treated and untreated cellulose-coated beads and their methods of use. In some embodiments, the treatment is with NaOH. In some embodiments, the methods of use are for normalizing a nucleic acid library or extracting cell-free DNA (cfDNA).

Embodiment 1. A method of extracting cell-free DNA (cfDNA) from a sample comprising the steps of:
a. combining:
   i. the sample;
   ii. a proteinase; and
   iii. treated cellulose-coated beads, wherein the treated cellulose-coated beads are prepared by a treatment with NaOH;
b. binding the cfDNA to the cellulose-coated beads in a binding buffer;
c. washing the bound cfDNA with a wash buffer; and
d. eluting the bound cfDNA with a resuspension buffer.
   Embodiment 2. The method of embodiment 1, further comprising washing the bound cfDNA with an ethanol wash after the washing step and before the eluting step.
   Embodiment 3. The method of embodiment 1 or embodiment 2, wherein the sample is blood.
   Embodiment 4. The method of embodiment 1 or embodiment 2, wherein the sample is plasma.
   Embodiment 5. The method of embodiment 1, further comprising performing size selection after step (d) comprising the steps of:
e. binding the eluted cfDNA to carboxylate beads; and
f. eluting the bound cfDNA from the carboxylate beads with a resuspension buffer.
   Embodiment 6. The method of any one of embodiments 1-5, wherein the proteinase and the treated cellulose-coated beads are combined in the binding buffer and the sample is applied to the binding buffer.
   Embodiment 7. The method of any one of embodiments 1-5, wherein the proteinase is combined with the sample before the treated cellulose-coated beads are combined with the sample.
   Embodiment 8. The method of any one of embodiments 1-7, wherein the proteinase is proteinase K.
   Embodiment 9. The method of any one of embodiments 1-8, wherein the method further comprises treating cellulose-coated beads with NaOH before step (a).
   Embodiment 10. The method of embodiment 1-9, wherein the treated cellulose-coated beads have an increased binding capacity when compared to untreated cellulose-coated beads.
   Embodiment 11. The method of embodiment 10, wherein the binding capacity of the treated cellulose-coated beads is 290-330 ng/μL.
   Embodiment 12. The method of embodiment 10 or embodiment 11, wherein the binding capacity of the treated cellular-coated beads is 1.5-2.5 times higher when compared to untreated cellulose-coated beads.
   Embodiment 13. The method of any one of embodiments 1-12, wherein the increased binding capacity of the treated cellular-coated beads is irreversible.
   Embodiment 14. The method of any one of embodiments 1-13, wherein the treatment with NaOH is selected to produce a desired extraction efficiency and/or a desired fragment size of cfDNA.
   Embodiment 15. The method of embodiment 14, wherein the desired extraction efficiency is 85% or more, 90% or more, or 95% or more of the amount of cfDNA in the sample.
   Embodiment 16. The method of embodiment 15, wherein the desired fragment size is 50-500 base pairs.
   Embodiment 17. The method of embodiment 16, wherein the desired fragment size is 140-180 base pairs.
   Embodiment 18. The method of any one of embodiments 5-17, wherein the performing size selection step comprises removing genomic DNA from the sample and retaining cfDNA.
   Embodiment 19. The method of any one of embodiments 5-18, wherein the performing size selection step comprises two rounds of binding to carboxylate beads, wherein the first round of binding is with carboxylate beads in a first buffer and the second round of binding is with carboxylate beads in a second buffer, wherein the first buffer has a higher concentration of PEG than the second buffer, wherein the first buffer preferentially binds genomic DNA; and wherein the second buffer preferentially binds cfDNA.
   Embodiment 20. A method of normalizing extracted cfDNA comprising:
a. combining a solution comprising cfDNA with cellulose-coated beads, optionally wherein the cellulose-coated beads are prepared by a treatment with NaOH;
b. binding the cfDNA to the cellulose-coated beads in a binding buffer;
c. washing the bound cfDNA with a wash buffer; and
d. eluting the bound cfDNA with a resuspension buffer to prepare normalized cfDNA.
   Embodiment 21. The method of embodiment 20, wherein the normalizing is performed with end-to-end automation.
   Embodiment 22. The method of embodiment 20 or 21, wherein the normalizing produces a desired level of yield of eluted cfDNA and/or a desired fragment size.
   Embodiment 23. The method of embodiment 22, wherein the desired fragment size is 75-300 base pairs.
   Embodiment 24. The method of embodiment 22, wherein the desired yield of eluted cfDNA is 5-50 ng.
   Embodiment 25. The method of any one of embodiments 20-24, wherein the extracted cfDNA is prepared using a method of any one of embodiments 1-19.
   Embodiment 26. The method of any one of embodiments 20-25, further comprising preparing a nucleic acid library from the eluted cfDNA.
   Embodiment 27. The method of any one of embodiments 1-26, wherein the cfDNA comprises fetal cfDNA and/or circulating tumor DNA.
   Embodiment 28. A method of normalizing a nucleic acid library comprising the steps of:
a. combining the library with cellulose-coated beads, optionally wherein the cellulose-coated beads are prepared by a treatment with NaOH;
b. binding the nucleic acid to the cellulose-coated beads in a binding buffer;
c. washing the bound nucleic acid with a wash buffer; and
d. eluting the bound nucleic acid with a resuspension buffer to prepare a normalized library.
   Embodiment 29. The method of embodiment 28, further comprising washing the bound nucleic acid with an ethanol wash after the washing step and before the eluting step.
   Embodiment 30. The method of embodiment 28 or 29, wherein the cellulose-coated beads are untreated and have a binding capacity of 150-180 ng/u L.
   Embodiment 31. The method of any one of embodiments 28-30, wherein the method further comprises treating cellulose-coated beads with NaOH before step (a).
   Embodiment 32. The method of embodiment 31, wherein the treated cellulose-coated beads have an increased binding capacity when compared to untreated cellulose-coated beads.
   Embodiment 33. The method of embodiment 31 or 32, wherein the binding capacity of the treated cellulose-coated beads is 290-330 ng/μL.
   Embodiment 34. The method of embodiment 32 or embodiment 33, wherein the binding capacity of the treated cellulose-coated beads is 1.5-2.5 times higher when compared to untreated cellulose-coated beads.
   Embodiment 35. The method of any one of embodiments 32-34, wherein the increased binding capacity of the cellulose-coated beads is irreversible.
   Embodiment 36. The method of any one of embodiments 28-35, wherein the treatment with NaOH is selected to produce a desired level of yield of eluted nucleic acid and/or a desired fragment size of the normalized library.
   Embodiment 37. The method of any one of embodiments 36, wherein the desired level of yield of eluted nucleic acid is 40% or more, 50% or more, 60% or more, 70% or more, or 80% or more of the nucleic acid library.
   Embodiment 38. The method of any one of embodiments 28-37, wherein the normalizing separates reagents used for library preparation from the library.
   Embodiment 39. The method of any one of embodiments 28-38, wherein the normalizing produces a desired level of yield of eluted cfDNA and/or a desired fragment size.
   Embodiment 40. The method of embodiment 39, wherein the desired fragment size is 75-300 base pairs.
   Embodiment 41. The method of embodiment 39, wherein the desired yield of eluted cfDNA is 5-50 ng.
   Embodiment 42. The method of embodiment 28-41, wherein the method does not use carboxylate or streptavidin beads.
   Embodiment 43. The method of embodiment 42, wherein the method uses only cellulose-coated beads.
   Embodiment 44. The method of any one of embodiments 40-43, wherein the desired fragment size is 50-10000 base pairs.
   Embodiment 45. The method of embodiment 44, wherein the desired fragment size is 150-1000 base pairs.
   Embodiment 46. The method of any one of embodiments 28-45, wherein the normalizing is performed with treated cellulose-coated beads and the average fragment size comprised in the normalized library differs from the average fragment size comprised in the nucleic acid library before the normalizing by 15% or less, 10% or less, 5% or less, 4% or less, 3% or less, 2% or less, or 1% or less.
   Embodiment 47. The method of any one of embodiments 28-46, wherein the normalizing is performed with untreated cellulose-coated beads and the average fragment size comprised in the normalized library is 5% or more, 10% or more, 15% or more, 20% or more, 25% or more, or 30% or more larger than the average fragment size comprised in the nucleic acid library before the normalizing.
   Embodiment 48. The method of any one of embodiments 28-47, wherein the normalized library has a lower coefficient of variance compared to the library before the method of normalizing.
   Embodiment 49. The method of any one of embodiments 28-48, wherein the method of normalizing can be performed with end-to-end automation.
   Embodiment 50. The method of any one of embodiments 28-49, wherein the method of normalizing does not require calculation of the library concentration.
   Embodiment 51. The method of any one of embodiments 28-50, further comprising sequencing the normalized library.
   Embodiment 52. The method of embodiment 51, wherein the normalized library is amplified before the sequencing.
   Embodiment 53. The method of embodiment 51 or embodiment 52, wherein the normalized library is amplified and/or sequenced without determining the nucleic acid concentration of the normalized library.
   Embodiment 54. The method of embodiment 53, wherein the nucleic acid library is amplified before combining the library with cellulose-coated beads.
   Embodiment 55. The method of any one of embodiments 51-54, wherein the number of usable sequencing reads is higher for the normalized library as compared to the same nucleic acid library before the method of normalizing.
   Embodiment 56. The method of any one of embodiments 51-55, wherein the relative sequencing coverage of fragments with a GC bias of 62% or more or with a GC bias of 60%-70% is higher for the normalized library as compared to the same nucleic acid library before the method of normalizing.
   Embodiment 57. The method of any one of embodiments 51-56, wherein the normalized library has 10% or more, 20% or more, 30% or more, 40% or more, 50% or more, 60% or more, 70% or more, or 80% or more greater sequencing coverage of fragments with a GC bias of 62% or more or with a GC bias of 60%-70% as compared to the same nucleic acid library before the method of normalizing.
   Embodiment 58. The method of any one of embodiments 28-57, wherein the nucleic acid library comprises concatenated sequencing templates.
   Embodiment 59. The method of any one of embodiments 28-58, wherein the binding step is performed with mixing.
   Embodiment 60. The method of embodiment 59, wherein the mixing is performed using a bioshaker.
   Embodiment 61. The method of embodiment 59 or embodiment 60, wherein the mixing is performed for 30 minutes or more.
   Embodiment 62. The method of any one of embodiments 28-61, wherein the nucleic acid library comprises 10 ng/μL or less, 1 ng/μL or less, 500 pg/μL or less, 200 pg/μL or less, or 100 pg/μL or less of nucleic acid.
   Embodiment 63. The method of any one of embodiments 28-62, wherein the nucleic acid library has a volume of 100 μL or less, 50 μL or less, or 20 μL or less.
   Embodiment 64. The method of any one of embodiments 28-63, wherein the yield of eluted nucleic acid is similar over a range of pH of the binding buffer from pH 5-9.
   Embodiment 65. The method of any one of embodiments 28-64, wherein the library is prepared from cfDNA.
   Embodiment 66. The method of embodiment 65, wherein the cfDNA comprises fetal cfDNA and/or circulating tumor DNA.
   Embodiment 67. The method of any one of embodiments 28-66, wherein the library is a shotgun library.
   Embodiment 68. The method of any one of embodiments 28-66, wherein the nucleic acid library is prepared via the method of embodiment 26.
   Embodiment 69. A kit for extracting cfDNA from a sample or normalizing a nucleic acid library comprising:
a. cellulose-coated beads, optionally wherein the cellulose-coated beads are prepared by treating with NaOH;
b. a binding buffer;
c. a wash buffer; and
d. a resuspension buffer.
   Embodiment 70. The kit of embodiment 69, wherein the cellulose-coated beads are untreated and have a binding capacity of 150-180 ng/μL.
   Embodiment 71. The kit of embodiment 69, wherein the cellulose-coated beads are treated with NaOH and have an increased binding capacity when compared to untreated cellulose-coated beads.
   Embodiment 72. The kit of embodiment 71, wherein the binding capacity of the treated cellulose-coated beads is 290-330 ng/μL.
   Embodiment 73. The kit of embodiment 71 or embodiment 72, wherein the binding capacity of the treated cellulose-coated beads is 1.5-2.5 times higher when compared to untreated cellulose-coated beads.
   Embodiment 74. The kit of any one of embodiments 71-73, wherein the increased binding capacity of the treated cellulose-coated beads is irreversible.
   Embodiment 75. The kit of any one of embodiments 69-74, wherein the kit is for extracting cfDNA from a sample and further comprises proteinase K and/or ethanol.
   Embodiment 76. The kit of any one of embodiments 69-75, wherein the kit is for normalizing a nucleic acid library and further comprises ethanol and/or reagents for preparing the library.
   Embodiment 77. The kit of embodiment 76, wherein the reagents for preparing the library comprise tagmentation or ligation reagents.
   Embodiment 78. The kit of embodiment 77, wherein the tagmentation reagents comprise bead-linked transposomes.
   Embodiment 79. The method or kit of any one of embodiments 1-78, wherein the treatment with NaOH is treatment with 0.2 to 1.0M NaOH for 6 to 48 hours.
   Embodiment 80. The method or kit of any one of embodiments 1-79, wherein the treatment with NaOH is treatment with 0.3 to 0.8M NaOH for 6 to 24 hours.
   Embodiment 81. The method or kit of any one of embodiments 1-80, wherein the treatment with NaOH is treatment with 0.5M NaOH for 6 to 8 hours.
   Embodiment 82. The method or kit of any one of embodiments 1-81, wherein the cellulose-coated beads have a diameter of 1 μm to 10 μm.
   Embodiment 83. The method or kit of any one of embodiments 1-82, wherein the cellulose-coated beads are magnetic.
   Embodiment 84. The method or kit of any one of embodiments 1-83, wherein the cellulose-coated beads comprise iron oxide.
   Embodiment 85. The method or kit of embodiment 84, wherein the cellulose-coated beads are 45%-55% iron oxide.
   Embodiment 86. The method or kit of any one of embodiments 1-85, wherein the particle suspension mass of the cellulose-coated beads is 100-150 mg/mL.
   Embodiment 87. The method or kit of any one of embodiments 1-86, wherein the particle suspension mass of the cellulose-coated beads is 110-140 mg/mL and/or the average density of the cellulose-coated beads is 3-4 g/cm3.
   Embodiment 88. The method or kit of any one of embodiments 1-87, wherein the binding buffer comprises a chaotropic agent.
   Embodiment 89. The method or kit of embodiment 88, wherein the chaotropic agent is guanidinium thiocyanate (GuSCN).
   Embodiment 90. The method or kit of embodiment 89, wherein the concentration of GuSCN is less than or equal to 7M.
   Embodiment 91. The method or kit of embodiment 90, wherein the concentration of GuSCN is 2-5M.
   Embodiment 92. The method or kit of any one of embodiments 1-91, wherein the binding buffer comprises PEG.
   Embodiment 93. The method or kit of embodiment 92, wherein the PEG comprises PEG200, PEG300, and/or PEG400.
   Embodiment 94. The method or kit of embodiment 92 or embodiment 93, wherein the PEG concentration is 30%-40% (weight/volume).
   Embodiment 95. The method or kit of embodiment 92 or embodiment 93, wherein the PEG concentration is 30% or less (weight/volume).
   Embodiment 96. The method or kit of embodiment 95, wherein the PEG concentration is 10%-30% (weight/volume).
   Embodiment 97. The method or kit of any one of embodiments 1-96, wherein the binding buffer comprises Tris and/or a detergent.
   Embodiment 98. The method or kit of embodiment 97, wherein the Tris concentration is 15-25 mM.
   Embodiment 99. The method or kit of embodiment 96 or embodiment 97, wherein the Tris is pH 6.5 to pH 7.
   Embodiment 100. The method or kit of any one of embodiments 97-99, wherein the detergent is Tween-20.
   Embodiment 101. The method or kit of embodiment 100, wherein the Tween-20 concentration is 0.1%-0.2% (weight/volume).
   Embodiment 102. The method or kit of any one of embodiments 1-101, wherein the binding buffer comprises PEG and GuSCN.
   Embodiment 103. The method or kit of embodiment 102, wherein the binding buffer comprises:
a. 30%-40% (weight/volume) of PEG200, PEG300, and/or PEG400; and
b. 2-5M GuSCN.
   Embodiment 104. The method or kit of embodiment 102, wherein the binding buffer comprises:
a. 30% or less (weight/volume) of PEG200, PEG300, and/or PEG400; and
b. 2-5M GuSCN.
   Embodiment 105. The method or kit of embodiment 104, wherein the binding buffer comprises:
a. 10%-30% (weight/volume) of PEG200, PEG300, and/or PEG400; and
b. 2-5M GuSCN.
   Embodiment 106. The method or kit of any one of embodiments 102-105, wherein the binding buffer comprises 3M or more GuSCN.
   Embodiment 107. The method of any one of embodiments 28-68, wherein the binding buffer is used for normalizing a nucleic acid library and the normalized library has a greater number of fragments with a GC bias of 62% or more or with a GC bias of 60%-70% in comparison to a normalized library wherein the binding buffer comprises more than 30% (weight/volume) of PEG200, PEG300, and/or PEG400.
   Embodiment 108. The method of embodiment 28-68, wherein the binding buffer is used for normalizing a nucleic acid library and the normalized library has 10% or more, 20% or more, 30% or more, 40% or more, 50% or more, 60% or more, 70% or more, or 80% or more fragments having a GC bias of 62% or more or with a GC bias of 60%-70% in comparison to a normalized library wherein the binding buffer comprises more than 30% (weight/volume) of PEG200, PEG300, and/or PEG400.
   Embodiment 109. The method or kit of any one of embodiments 1-108, wherein the binding buffer comprises PEG, Tris, GuSCN, and Tween-20.
   Embodiment 110. The method or kit of embodiment 109, wherein the binding buffer comprises:
a. 30%-40% (weight/volume) of PEG200, PEG300, and/or PEG400;
b. 15-25 mM Tris pH 6.5 to pH 7;
c. 2-5M GuSCN; and
d. 0.1%-0.2% (weight/volume) Tween-20.
   Embodiment 111. The method or kit of any one of embodiments 1-110, wherein the binding buffer has a viscosity of 8.0-8.5 mPa*s at 25° C.
   Embodiment 112. The method or kit of any one of embodiments 1-111, wherein the binding buffer has a conductivity of 49-51 ms/cm at 25° C.
   Embodiment 113. The method or kit of any one of embodiments 1-112, wherein the binding buffer has a refractive index of 70%-80% Brix at 25° C.
   Embodiment 114. The method or kit of any one of embodiments 1-113, wherein the wash buffer comprises a chaotropic agent.
   Embodiment 115. The method or kit of embodiment 114, wherein the chaotropic agent is GuSCN.
   Embodiment 116. The method or kit of embodiment 115, wherein the concentration of GuSCN is less than 3.5M.
   Embodiment 117. The method or kit of embodiment 116, wherein the concentration of GuSCN is 1-3M.
   Embodiment 118. The method or kit of any one of embodiments 1-117, wherein the wash buffer comprises PEG.
   Embodiment 119. The method or kit of embodiment 118, wherein the PEG comprises PEG200, PEG300, and/or PEG400.
   Embodiment 120. The method or kit of embodiment 118 or 119, wherein the PEG concentration is 15%-20% (weight/volume).
   Embodiment 121. The method or kit of any one of embodiments 1-120, wherein the wash buffer comprises Tris and/or a detergent.
   Embodiment 122. The method or kit of any one of embodiments 1-121, wherein the Tris concentration is 8-12 mM.
   Embodiment 123. The method or kit of embodiment 121 or embodiment 122, wherein the Tris is pH 6.5 to pH 7.
   Embodiment 124. The method or kit of any one of embodiments 121-123, wherein the detergent is Tween-20.
   Embodiment 125. The method or kit of embodiment 124, wherein the Tween-20 concentration is 0.05%-0.1% (weight/volume).
   Embodiment 126. The method or kit of any one of embodiments 1-125, wherein the wash buffer comprises PEG and GuSCN.
   Embodiment 127. The method or kit of embodiment 126, wherein the wash buffer comprises:
a. 15%-20% (weight/volume) PEG200, PEG300, and/or PEG400; and
b. 1-3M GuSCN.
   Embodiment 128. The method or kit of embodiment 126 or 127, wherein the wash buffer comprises PEG, Tris, GuSCN, and Tween-20.
   Embodiment 129. The method or kit of any one of embodiments 1-128, wherein the wash buffer comprises:
a. 15%-20% (weight/volume) PEG200, PEG300, and/or PEG400;
b. 8-12 mM Tris;
c. 1-3M GuSCN; and
d. 0.05%-0.1% (weight/volume) Tween-20.
   Embodiment 130. The method or kit of any one of embodiments 1-129, wherein the wash buffer has a viscosity of 1.8-2.0 mPa*s at 25° C.
   Embodiment 131. The method or kit of any one of embodiments 1-130, wherein the wash buffer has a conductivity of 85-90 ms/cm at 25° C.
   Embodiment 132. The method or kit of any one of embodiments 1-131, wherein the wash buffer has a refractive index of 40-45% Brix at 25° C.
   Embodiment 133. A method of preparing cellulose-coated beads for binding nucleic acid comprising:
a. incubating the beads with a NaOH solution; and
b. washing the beads with a wash buffer.
   Embodiment 134. The method of embodiment 133, wherein the treated cellulose-coated beads have an increased binding capacity when compared to untreated cellulose-coated beads.
   Embodiment 135. The method of embodiment 133 or embodiment 134, wherein the binding capacity of the treated cellulose-coated beads is 290-330 ng/μL.
   Embodiment 136. The method of embodiment 134 or 135, wherein the binding capacity of the treated cellulose-coated beads is 1.5-2.5 times higher when compared to untreated cellulose-coated beads.
   Embodiment 137. The method of any one of embodiments 133-136, wherein the increased binding capacity of the treated cellulose-coated beads is irreversible.
   Embodiment 138. The method of any one of embodiments 133-137, wherein the NaOH solution comprises 0.2 to 1.0M NaOH.
   Embodiment 139. The method of embodiment 138, wherein the NaOH solution comprises 0.3 to 0.8M NaOH.
   Embodiment 140. The method of embodiment 139, wherein the NaOH solution comprises 0.5M NaOH.
   Embodiment 141. The method of any one of embodiments 133-140, wherein the incubating step is at least 60 minutes.
   Embodiment 142. The method of embodiment 141, wherein the incubating step is from 6 to 48 hours.
   Embodiment 143. The method of embodiment 142, wherein the incubating step is from 8 to 24 hours.
   Embodiment 144. The method of embodiment 141, wherein the incubating step is from 1 to 12 hours.
   Embodiment 145. The method of embodiment 143, wherein the NaOH solution comprises 0.5M NaOH and the incubating step is 8 hours.
   Embodiment 146. The method of any one of embodiments 133-145, wherein the washing step is performed with 2 or more rounds of wash buffer.
   Embodiment 147. The method of any one of embodiments 133-146, further comprising a step of storing the beads in a storage buffer.
   Embodiment 148. The method of any one of embodiments 133-147, wherein the cellulose-coated beads have a diameter of 1 μm to 10 μm.
   Embodiment 149. The method of any one of embodiments 133-148, wherein the cellulose-coated beads are magnetic.
   Embodiment 150. The method of any one of embodiments 133-149, wherein the cellulose-coated beads comprise iron oxide.
   Embodiment 151. The method of any one of embodiments 133-150, wherein the cellulose-coated beads are 45%-55% iron oxide.
   Embodiment 152. The method of any one of embodiments 133-151, wherein the particle suspension mass of the cellulose-coated beads is from 100-150 mg/mL.
   Embodiment 153. The method of embodiment 152, wherein the particle suspension mass of the cellulose-coated beads is from 110-140 mg/mL.

Additional objects and advantages will be set forth in part in the description which follows, and in part will be obvious from the description, or may be learned by practice. The objects and advantages will be realized and attained by means of the elements and combinations particularly pointed out in the appended embodiments.

It is to be understood that both the foregoing general description and the following detailed description are exemplary and explanatory only and are not restrictive of the claims.

The accompanying drawings, which are incorporated in and constitute a part of this specification, illustrate one (several) embodiment(s) and together with the description, serve to explain the principles described herein.

### BRIEF DESCRIPTION OF THE DRAWINGS

FIGS. 1A and 1B show cell-free DNA (cfDNA) extraction yield with cellulose-coated beads versus comparator beads (Bioo Scientific beads comprised in NextPrep-Mag Kit 3825-01). Using untreated cellulose-coated beads (A), the extraction yield was lower than comparator beads. After NaOH treatment of cellulose-coated beads (B), the extraction of cfDNA was equivalent to comparator beads. In this preliminary experiment, the NaOH treatment was 1M NaOH for 60 hours.

FIGS. 2A and 2B shows cfDNA fragment size after extraction with cellulose-coated beads versus comparator beads. Using untreated cellulose-coated beads (A), the fragment size extracted was larger than comparator beads. After NaOH treatment of cellulose-coated beads (B), the fragment size of extracted cfDNA was equivalent to comparator beads. In this preliminary experiment, the NaOH treatment was 1M NaOH for 60 hours.

FIG. 3 shows results of cfDNA extraction from artificial plasma for cellulose-coated beads treated with 0.5M or 1M NaOH for various times. Shaded boxes indicate similar results to comparator beads. NES/μL=usable sequencing reads per volume of library input (TransferVol); FF=fetal fraction; NCV_Y=normalized chromosome_Y, i.e., Y sequencing count; Cov=coverage; FragSizeDist=fragment size distribution; Bin_MAD=metric for sequencing noise compared to the known baseline with a larger value indicating more noise, in this case the known baseline was established using residual plasma aliquots collected from Illumina VeriSeq NIPT.

FIG. 4 shows that cellulose-coated beads treated for 6 hours with 0.5M NaOH or 1M NaOH generally showed comparable results to comparator beads, with shaded boxes indicating similar results to comparator beads.

FIGS. 5A-5D show non-excluded sites (A), fetal fraction (B), extracted DNA fragment size distribution (FragSizeDist, C), and usable sequencing reads per volume of library input (NES/μL, D) were all comparable for comparator beads or beads treated with 1M or 0.5M NaOH.

FIG. 6 shows that normalized chromosome Y values are comparable after extraction with either cellulose-coated beads treated with 0.5M NaOH or comparator beads, with results being less consistent to the comparator for beads treated with 1M NaOH.

FIG. 7 shows that and non-excluded sites2Tags (a measure of reads useful for NIPT analysis per the total reads) are comparable after extraction with either cellulose-coated beads treated with 0.5M NaOH or comparator beads, with results being less consistent to the comparator for beads treated with 1M NaOH.

FIG. 8 shows cfDNA extraction yield relative to comparator beads for cellulose-coated beads treated with different concentrations of NaOH for different time periods. These data highlight that the relative yield can be adjusted by using beads that were subjected to different treatments.

FIG. 9 shows cfDNA extraction yield relative to comparator beads for cellulose-coated beads treated with 0.5M NaOH for various treatment times.

FIG. 10 shows an overview of a standard workflow to extract cfDNA (such as fetal cfDNA or circulating tumor DNA, ctDNA). The current standard protocol includes plasma preparation with a Streck tube. After library preparation, gene mutations can be detected by next generation sequencing (NGS). The icon with the number 1 inside a star shape notes where magnetic bead extraction of cfDNA is performed. The icon with the number 2 inside a star shape notes the step of manual plasma preparation that may be removed from a protocol if cfDNA is directly extracted from blood without first preparing plasma.

FIG. 11 shows protocol steps for cfDNA extraction from plasma or directly from blood (with a first version of a direct blood extraction). While plasma preparation is performed manually, the other steps in the workflow (WF) can be automated. As such, the protocol to prepare cfDNA directly from blood can be fully automated.

FIGS. 12A and 12B show that cfDNA extraction with treated cellulose-coated beads (A) was generally comparable to results with a comparator of Qiagen QIAmp Circulating Nucleic Kit (CNA) (B), with similar peak fragment sizes.

FIGS. 13A and 13B show that spiked cfDNA can be extracted using a bead-based protocol for both high and low DNA inputs (A, 100 ng and 10 ng) and from both blood and water (B) for a 10 ng/ml sample.

FIGS. 14A and 14B show that direct extraction of spiked recombinant DNA from blood (A) shows similar results to cfDNA extraction from plasma (B).

FIGS. 15A and 15B show a first version of a fully automated workflow (WF) for cfDNA extraction from blood that includes size selection using Illumina Tune Beads (ITB, A) and a shortened fully automated workflow (B).

FIGS. 16A and 16B show steps for cfDNA extraction from blood (A) or plasma (B) using NaOH-treated cellulose-coated beads. ProK=proteinase K; Sup=supernatant.

FIG. 17 shows a representative size selection protocol with carboxylate beads (e.g., Illumina Tune Beads, ITB) that may be used with cfDNA extraction from blood. gDNA=genomic DNA; HMW=high molecular weight DNA.

FIG. 18 shows that bead-based normalization recovery efficiency and yield (ng of DNA recovered) were constant over a range of DNA inputs.

FIG. 19 shows improved recovery efficiency when normalizing in a smaller volume (20 μL versus 100 μL).

FIG. 20 shows that repeated mixing (e.g., “repeatedly” mixing with a bioshaker) improves recovery efficiency over a single mix with a pipettor (“once”).

FIG. 21 shows the effect of decreasing mixing time on cfDNA extraction.

FIG. 22 shows that as the number of beads per reaction is lowered, the DNA captured levels off at a proportionally lower amount. For example, more DNA can be captured with 0.1 μL beads as compared to 0.025 μL beads.

FIG. 23 shows the effect of time of mixing on total DNA capture for the bead-based extraction protocol.

FIG. 24 presents results of coefficient of variance (CV) for input DNA versus output DNA after bead-based normalization with NaOH-treated beads.

FIG. 25 shows that fragment size was similar for manual normalization and bead-based normalization with NaOH-treated beads.

FIG. 26 presents a comparison of protocols for manual normalization and bead-based normalization.

FIG. 27 shows an overview of the present method for bead-based normalization with treated cellulose-coated beads. In some methods, two washes with wash buffer may be used before an ethanol wash. Alternatively, a large wash volume (such as 200 μL for a 96-well plate) may be used

FIG. 28 shows results for control (manual) normalization versus the present bead-based normalization with NaOH-treated beads, with a comparison between results for two different users. As shown in the figure, different aliquots of the same sample (shown in dots) as well as spread of data were similar for the two users (i.e., the comparison of the values for the two users were close to 1).

FIG. 29 shows the amount of DNA recovered with different types of beads. Treated cellulose-coated beads refer to beads treated with 0.5M NaOH for 6 hours. “HTC beads” refer to comparator Maxwell® HT Cellulose beads (Promega). Untreated cellulose-coated beads showed lower DNA recovery than treated cellulose-coated beads, and the recovery with untreated beads could be increased with a greater number of beads.

FIG. 30 shows that the pg/μL output of a normalization protocol with untreated cellulose-coated beads following 1 or 2 washes with wash buffer.

FIG. 31 shows quantification of yield from a normalization protocol with untreated cellulose-coated beads and a DNA input of 25 μg or 100 μg with a single wash of 0, 100, 150, or 200 μL. Qubit Quants=quantifications using the Qubit™ dsDNA HS Assay according to manufacturer protocol.

FIG. 32 shows that relatively small changes in pipetting volume can impact DNA quantification when untreated cellulose-coated beads are used for normalization of 25 ng or 100 ng samples. The bead/buffer volume unit is μL.

FIG. 33 shows the effect of PEG content on normalization results with untreated cellulose-coated beads.

FIG. 34 shows normalization results with untreated cellulose-coated beads when different types of PEG are used.

FIGS. 35A and 35B show effect of bead amount on normalization recovery. Different percent solutions of untreated cellulose-coated beads had relatively linear relationship to normalization yield (A) with changes in bead amount aligning with changes in normalization yield (B).

FIG. 36 shows results with different mixing times (from 10 minutes to 30 minutes) for 25 and 100 ng DNA sample inputs and untreated cellulose-coated beads. “Mixing” results shown are with a biomixer. “Mix Once,” “Mix Twice,” and “Mix Thrice” results are with manual mixing with a pipetter.

FIG. 37 shows data on successful normalization with untreated cellulose-coated beads across a range of different samples with a DNA target size from 5 kb-10 kb or 25 kb-60 kb.

FIG. 38 shows data on successful elution of DNA from untreated cellulose-coated beads, with little increase in elution efficiency with longer elution times.

FIG. 39 shows fragment size of unnormalized libraries (raw sample) or libraries after normalization with either NaOH-treated beads or untreated beads. Two libraries were compared, one with a relatively smaller size (6330 base pairs) and one with a relatively larger size (8030 base pairs). For both libraries, the fragment size of the library was larger after normalization with untreated beads.

FIG. 40 shows fragment size after PCR amplification of a library with relatively smaller fragment size, showing that normalization with untreated beads led to a larger fragment size than normalization with NaOH-treated beads.

FIG. 41 shows fragment size after PCR amplification of a library with relatively smaller fragment size, showing that the fragment size of the normalized library with untreated beads was larger than the raw sample.

FIG. 42 shows fragment size after PCR amplification of a library with relatively smaller fragment size, showing that normalization with untreated beads led to fragment size similar to that of the raw sample.

FIG. 43 shows fragment size after PCR amplification of a library with relatively large fragment size, showing that normalization with untreated beads led to a larger fragment size than normalization with NaOH-treated beads.

FIG. 44 shows fragment size after PCR amplification of a library with relatively larger fragment size, showing that the fragment size of the normalized library with untreated beads was larger than the raw sample.

FIG. 45 shows fragment size after PCR amplification of a library with relatively larger fragment size, showing that normalization with untreated beads led to fragment size similar to that of the raw sample.

FIG. 46 shows differences in sequencing data between a library normalized using the present methods versus a raw (unnormalized) library. As shown in the top box, normalized library samples had a significant number of reads with high GC content (i.e., GC bias). However, many samples with high GC content were missing from the raw library as shown in the bottom box. The raw input library has a relatively flat X-axis for GC bias, indicating that it is not biased to include samples with high GC content.

FIGS. 47A and 47B show effects of normalization with NaOH-treated beads in a binding buffer with 3M GuSCN. As used herein, “overtagged” refers to a library comprising fragments of relatively smaller size, which can be achieved with more tagging reactions by transposase enzymes on genomic DNA (i.e., higher number of transposases or longer reaction time). In this experiment, an overtagged library has an average fragment size of approximately 6 kb in comparison to standard library fragment size under these conditions of approximately 8 kb. (A) In the presence of 3M GuSCN, the fragment size of the libraries after normalization by manual method or by using NaOH-treated cellulose-coated beads was similar. (B) GC bias was significantly higher for a library after normalization with treated beads and binding buffer with 3M GuSCN as compared the same library after manual normalization.

FIGS. 48A and 48B show effects of normalization with untreated beads in a binding buffer with 3M GuSCN. (A) In the presence of 3M GuSCN, the fragment size of the libraries post-PCR after normalization by manual method or by using untreated cellulose-coated beads had more overlap (i.e., more similarly sized fragments) than when lower GuSCN concentrations were used. (B) GC bias was relatively high for libraries normalized with untreated beads whether binding buffer was LRB-control (4M GuSCN, 35% PEG300 (w/v), Tris 20 mM, pH6.8, Tween 20 (w/v) 0.02%) or LRB-more-PEG-Guan-2 (7M GuSCN, 35% PEG300 (w/v), Tris 20 mM, pH6.8, Tween 20 (w/v) 0.02%), with the higher GuSCN concentration causing a small shift towards more GC bias.

FIGS. 49A and 49B show effects of normalization with NaOH-treated beads in a binding buffer with less than 3M GuSCN. (A) With a binding buffer comprising less than 3M GuSCN, the fragment size of the libraries after normalization by manual method or by using NaOH-treated cellulose-coated beads was more similar (i.e., more overlay in the fragment size curves). (B) GC bias was retained under these conditions.

FIG. 50 shows that DNA capture increased with higher percentage of PEG8000 (weight/volume) in the binding buffer, while GC bias decreased with higher concentrations of PEG8000. “Control” refers to a library with a fragment size of approximately 8 Kb, and “overtagged” refers to a library with a fragment size of approximately 6 Kb. For both library types, binding buffers with PEG8000 concentrations of 7.5% or greater captured more DNA but had decreased GC bias. Similar trends were seen for untreated and NaOH-treated cellulose-coated beads (data not shown).

FIG. 51 shows that higher GuSCN content in the binding buffer did not significantly change DNA capture amount but did increase GC bias for both untreated and NaOH-treated beads. This effect of GuSCN on GC bias was not seen with manual normalization.

FIG. 52 shows that DNA capture with untreated beads increased with higher percentage of PEG300 (weight/volume) in the binding buffer, while GC bias decreased with higher concentrations of PEG300. Most GC bias differences from manual normalization were seen with binding buffer having 10%-30% PEG (weight/volume) with much less GC bias with binding buffer having 30-35% PEG.

FIG. 53 shows changes in GC bias of untreated beads using different binding buffers, with large differences shown in the box highlighting that GC heavy regions normally have extremely low coverage. Thus increased GC bias can have a large effect on sequencing coverage of GC-heavy regions.

FIGS. 54A and 54B show issues may arise with library preparation from non-normalized ctDNA (A), while normalization prior to library preparation can help to improve success (B).

FIG. 55 shows representative results of DNA yield after binding to a mixture of carboxylated magnetic beads (ITB beads) and silica magnetic beads. DNA binding is always linear and never reaches a plateau.

FIG. 56 shows DNA binding to silica magnetic beads or carboxyl beads reached a plateau that was higher than the desired yield. AMO beads are AMO-Mag™ beads from AMO LifeScience, and Apostle beads are Apostle MiniEnrich Carboxyl Beads from Apostle.

FIGS. 57A and 57B show cfDNA binding to cellulose-coated beads, as presently described, reached a plateau at the desired level of DNA yield (A) with a desired insert size (B).

FIGS. 58A-58C show results of cfDNA normalization using an automated bead-based normalization (BBN) protocol, such as one described in Table 9. The desired yield levels of less than 35 ng (A) and fragment size (B) were seen with the automated protocol. Also shown are results with spike-ins of high molecular-weight DNA, which is similar to known impurities in plasma samples (C). Lanes B1 and C1 of panel C show control samples without any extraction and a % cfDNA of 55% (shown at bottom of lanes). In contrast, the other lanes of panel C show results after extraction with the presently described cellulose coated beads, which reduced the yield of high-molecular DNA and increased the % cfDNA to 77% or greater based on this reduction in impurities.

FIGS. 59A and 59B show issues may arise with sequencing from non-normalized libraries as sequencing data may be biased towards certain samples (A), while normalized libraries can provide better sequencing results with more consistent sequencing data across samples (B).

FIG. 60 shows workflows for purification, size selection, and normalization of libraries, such as shotgun libraries. Such shotgun libraries may be prepared from cfDNA, such as ctDNA. The current standard for purification, size selection, and normalization of shotgun libraries using multiple beads (process 1). In contrast, the presently described cellulose-coated beads allow for a single bead to perform purification, size selection, and normalization (process 2). Also shown is that carboxylated beads (process 3) cannot be used to perform a combined method of purification, size selection, and normalization, as discussed above for FIG. 55. LNA1=library normalization buffer; LNB1=library normalization beads (streptavidin magnetic beads with pre-attached biotinylated DNA); LNW1=library normalization wash; SPRI=solid phase reversible immobilization.

FIGS. 61A and 61B show results from library normalization of a shotgun library. Representative cfDNA library yield with normalization across a range of input DNA amounts are shown (A). Similar post-normalization output peak fragment sizes were seen for 50 ng-350 ng DNA inputs, indicating uniform normalization performance over a range of DNA inputs (B).

FIGS. 62A and 62B show eluted library concentrations with purification/size selection and normalization of a library with cellulose-coated beads (A) or with a 2-bead method of purification/size selection with SPRI beads followed by normalization with LNB1 beads (B).

FIGS. 63A-63C show sequencing metrics for purification/size selection and normalization with cellulose-coated beads as compared to a 2-bead method of purification/size selection with SPRI beads followed by normalization with LNB1 beads. Sequencing results include gene scaled MAD (A), mean family depth (B), and percent duplex family measurements (C). MAD=median of absolute deviations; pooled cfDNA 145=cfDNA extracted from a human plasma pool designated as #145; SeraCare 0.5%=Seraseq ctDNA Complete Mutation Mix AF 0.5% (SeraCare Life Sciences material number 0710-0531). More details on measurements used in FIGS. 63A-64B are provided in Table 10 below.

FIGS. 64A and 64B show additional sequencing metrics for purification/size selection and normalization with cellulose-coated beads as compared to a 2-bead method of purification/size selection with SPRI beads followed by normalization with LNB1 beads. Sequencing results include median exon coverage (A) and PCT read enrichment (B).

FIGS. 65A and 65B show that no GC bias difference was observed for sequencing data from cellulose-coated beads as compared to the 2-bead method of purification/size selection with SPRI beads followed by normalization with LNB1 beads for pooled cfDNA samples (A) and SeraCare samples (B).

### DESCRIPTION OF THE EMBODIMENTS

### I. Treated Beads

As described herein, cellulose-coated beads may be treated, such as with NaOH. In some embodiments, these treatments may alter the cellulose-coated beads, such as by increasing binding area or binding capacity. In some embodiments, cellulose-coated beads may be treated under relatively mild conditions, such as with concentrations of 1M NaOH or less. These treated beads may be used for methods comprising one or more step of binding of nucleic acids, such as DNA, to the beads.

### A. Beads Comprising Cellulose

In some embodiments, beads comprise cellulose. As used herein, “cellulose beads” or “cellulose-coated beads” may comprise other components, such as a magnetic substance. In some embodiments, the beads comprise a cellulose coating on top of a bead core that is not cellulose. In some embodiments, a bead core comprises a magnetic substance, such as iron, iron oxide, or copper.

In some embodiments, the beads comprise microporous cellulose. In some embodiments, the cellulose is a resin or matrix on the surface of a bead comprising a magnetic substance. In some embodiments, a solid bead is encapsulated with cellulose.

In some embodiments, nucleic acids, such as DNA, can bind to cellulose comprised in a bead. As described in Tan and Yiap Journal of Biomedicine and Biotechnology 2009: Article ID 574398 (2009), nucleic acids bound to a cellulose matrix or resin can be washed with a wash buffer and then contacted with a suitable elution buffer to separate the desired nucleic acid from the cellulose. In some embodiments, these characteristics of cellulose binding to nucleic acids is utilized in methods of normalizing a library or extracting cfDNA.

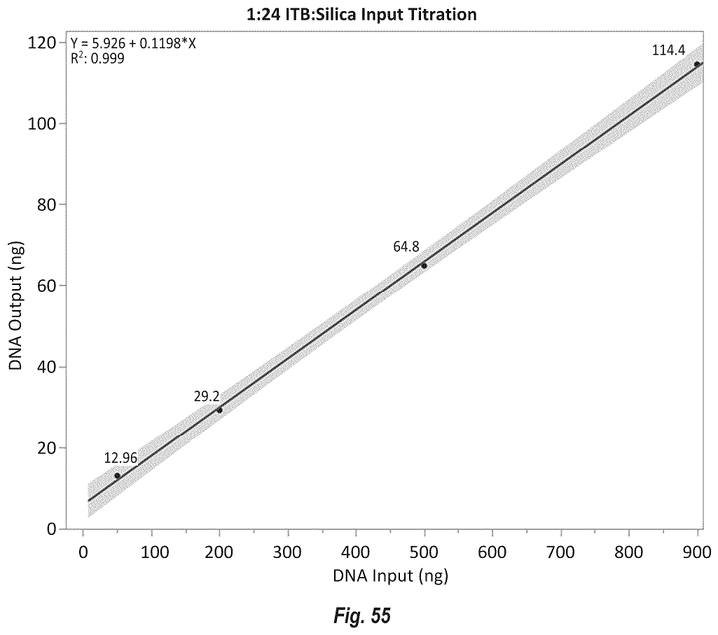

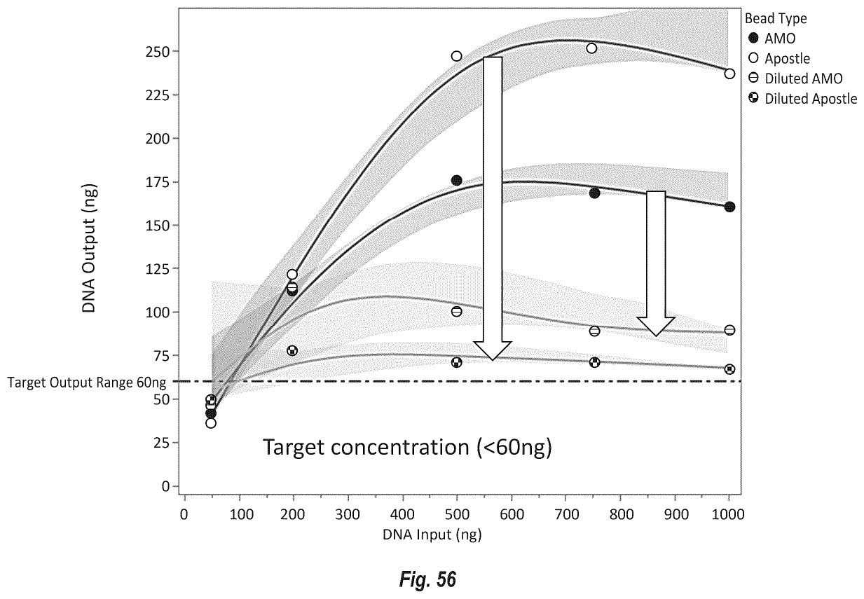

In some embodiments, the characteristics of beads comprising cellulose improve results as compared to other types of beads with DNA-binding characteristics. For example, FIG. 55 shows results with a mixture of carboxylated magnetic beads (such as SPRI beads) and silica magnetic beads (wherein the silica beads are used as “dummy beads” for operational benefits and do not bind DNA in the buffer used). However, DNA binding is always linear and never reaches a plateau. Further, FIG. 56 shows that silica magnetic beads (AMO beads and Apostle beads) showed a plateau for DNA binding, but the high binding capacity and small bead size kept the amount of DNA yield from binding too high (i.e., above the target concentration of less than 60 ng of DNA).

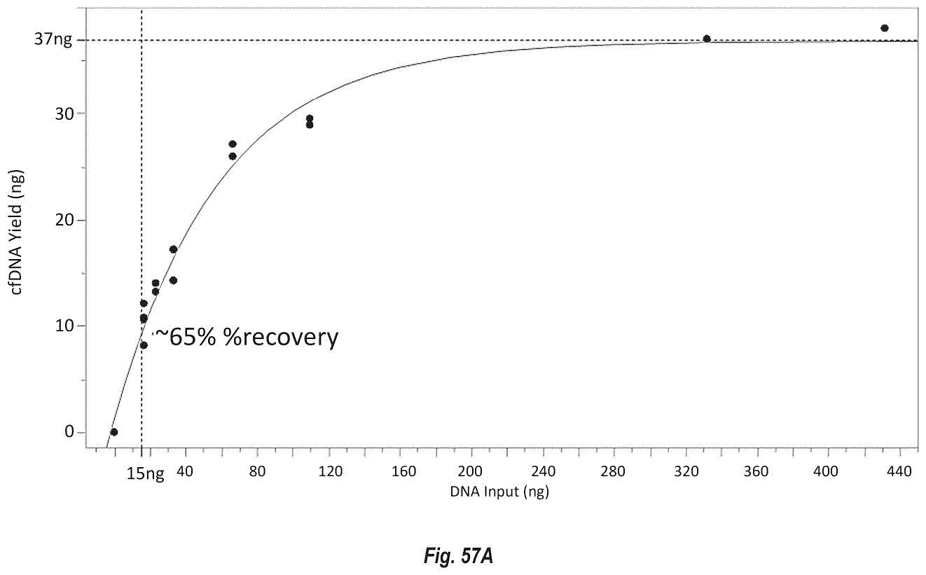

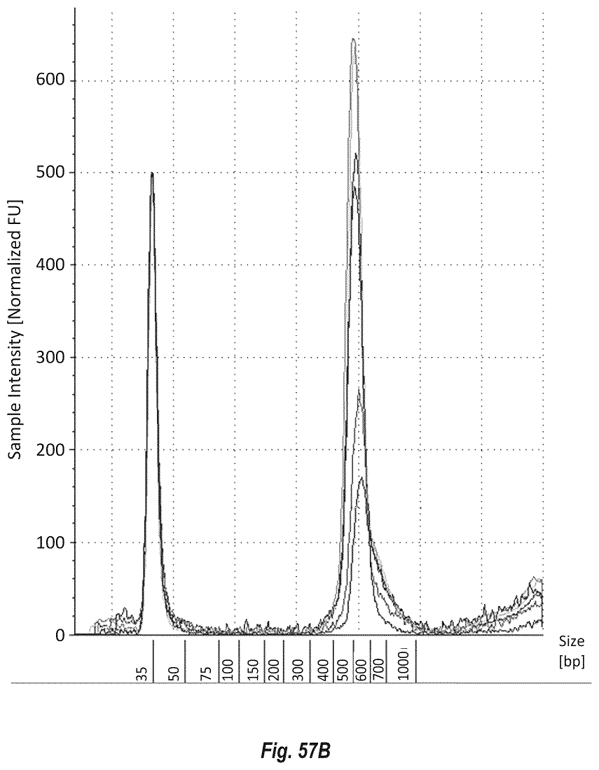

In contrast, the presently described cellulose-coated beads with a relatively large size (approximately 20 μm) and lower binding capacity allows for cfDNA binding yield in the desired range (FIG. 57A) with the desired insert size (FIG. 57B). These data on cellulose-coated beads and comparators are described in greater detail in Example 11.

In some embodiments, the cellulose-coated beads are 1 μm to 10 μm in diameter. In some embodiments, the cellulose-coated beads are 1 μm or greater in diameter, 5 μm or greater in diameter, 6 μm or greater in diameter, 7 μm or greater in diameter, 8 μm or greater in diameter, 9 μm or greater in diameter, or 10 μm or greater in diameter. As such, these beads may be generally larger than other types of beads used for DNA capture.

In some embodiments, the large size of the present cellulose-coated beads helps the user to visualize a pellet of the beads. As would be expected, the same number of large beads are much easier to visualize as compared to the same number of small beads. In this way, the present beads allow for an easy visualization of the bead pellet, even when only a relatively small number of beads are used.

In contrast, other commercial beads (such as SPRI beads) have a diameter of 0.9-1.05 μm. Users can have difficulty employing small beads, as they may not generate a visible pellet (such as after pelleting with centrifugation or with a magnet), especially when a small number of beads are used for normalization.

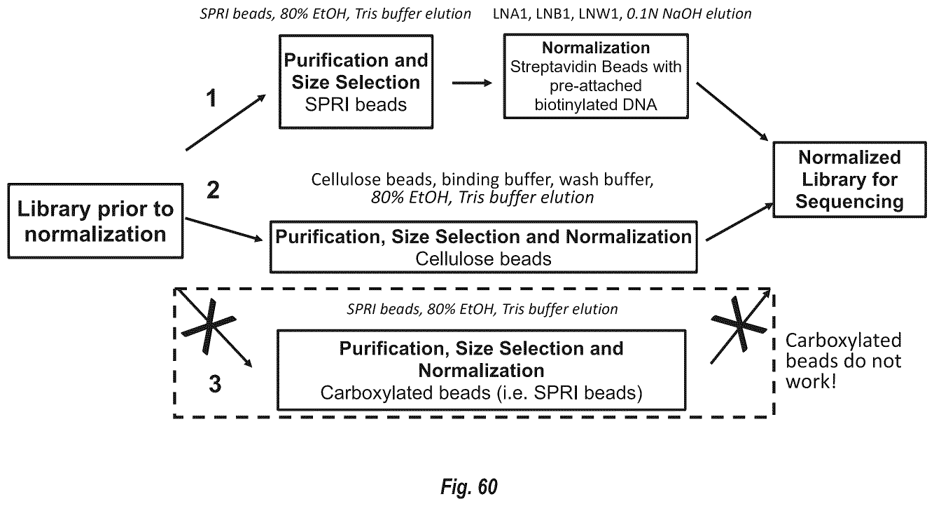

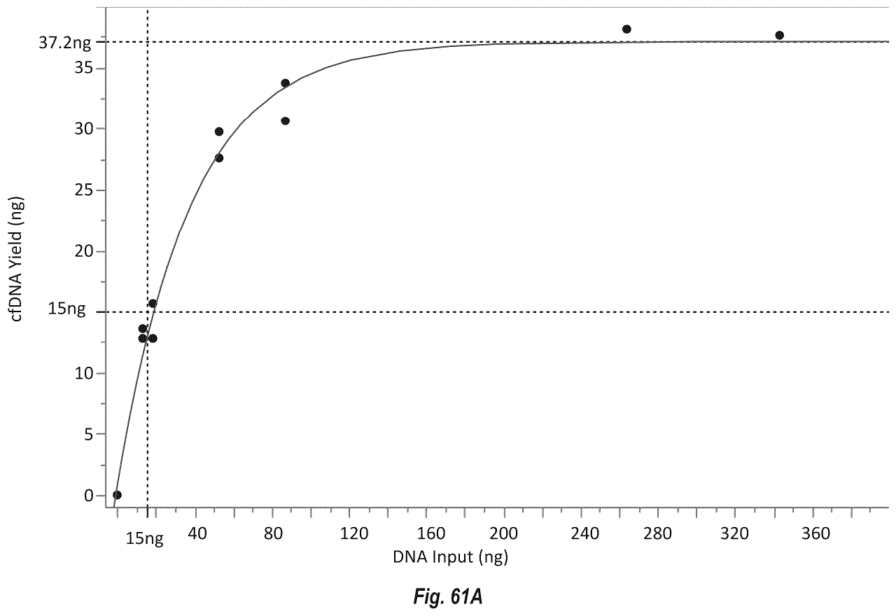

SPRI beads also have an extremely high binding capacity. For example, at least 7 g of nucleic acid can be bound by commercial AMPure XP reagents comprising SPRI beads (see, for example, Instructions for Use, Agencourt AMPure XP, Beckman Coulter, Document B37419AB, August 2016). Since SPRI beads have a very high DNA binding capacity along with a small size, this means an enormous dilution of beads may be necessary to try to normalize libraries with SPRI beads, and a user could have difficulty accurately estimating the proper dilution ratio to achieve such a small number of beads. Further, such a small number of SPRI beads may not produce a visible pellet, and a user cannot feel secure that they are pipetting a supernatant and not disturbing the beads. Further, SPRI beads alone were not appropriate for a method of purification, size selection, and normalization of shotgun libraries, while cellulose-coated described herein could be used in such a method. These results are described in Example 13 and FIGS. 60-65B.

The large size of the present cellulose-coated beads may have advantages as outlined herein, such as to promote a visible bead pellet for ease of use during wash steps. In addition, certain normalization methods described herein use untreated cellulose-coated beads with relatively low binding capacity, wherein more beads are used for a normalization method. Using more beads can help to allow for visualization on a pellet of the beads after preparing a bead pellet with centrifugation or use of a magnet.

In some embodiments, the untreated cellulose-coated beads (i.e. without treatment with NaOH) have a binding capacity of 150-180 ng/μL. In some embodiments, the binding capacity is measured with treated cellulose-coated beads in a solution of 113-138 mg of beads/mL.

Use of a higher number of large beads can significantly improve the ability of the user to visualize a bead pellet, such as when samples in a PCR plate are placed on a magnet.

In some embodiments, beads comprise silica. In some embodiments, beads are magnetic. In some embodiments, the beads comprise iron or iron oxide. In some embodiments, beads comprise copper. In some embodiments, the beads comprise iron or copper encapsulated by cellulose.

In some embodiments, the cellulose-coated beads comprise iron oxide. In some embodiments, the cellulose-coated beads are 45%-55% iron oxide.

In some embodiments, the properties of magnetic beads allow for rapid and efficient capture of beads for wash or elution steps. In some embodiments, magnetic beads are captured using a magnetic stand or plate magnet. A wide variety of magnetic stands are commercially available, such as MagneSphere® Technology Magnetic Separation Stand, PolyATtract® System 1000 Magnetic Separation Stand, and Deep-Well MagnaBot® 96 Magnetic Separation Device (Promega).

In some embodiments, beads are provided in a slurry comprising ethanol. Beads may be kept in suspension during methods of use with tube shakers or end-over-end mixers.

In some embodiments, the cellulose-coated beads are relatively heavy, as measured by their particle suspension mass. This heaviness can improve mixing protocols used within methods. In some embodiments, the particle suspension mass of the cellulose-coated beads is 100-150 mg/mL. In some embodiments, the particle suspension mass of the cellulose-coated beads is 110-140 mg/mL. In some embodiments, the average density of the beads is 3-4 g/cm3.

### B. Methods of Preparing Cellulose-Coated Beads

In some embodiments, beads comprising cellulose are treated. Cellulose resins have been previously described to be subject to swelling and dissolution in solvents, such as NaOH (see, for example, Budtova and Navard Cellulose 23 (1): 5-55 (2016)).

Further, other studies have shown that cellulose may fully dissolve in certain concentrations of NaOH, which destroys the nucleic acid binding capacity of a bead comprising cellulose (see, for example, Swensson et al., Cellulose 27:101-112 (2020)). Preliminary experiments with NaOH treatment as described herein showed the 2M NaOH dissolved the cellulose matrix on cellulose-coated beads (data not shown). Similarly, Swensson et al. found dissolution of microcrystalline cellulose in 2.3 M NaOH for 5 minutes, and Budtova and Navard described a range of studies inducing dissolution of various types of cellulose at concentrations above 2M. Thus, concentrations of 2M or above may induce dissolution of cellulose and reduce or eliminate the ability of treated beads to bind to nucleic acids.

In some embodiments, a method of preparing cellulose-coated beads for binding nucleic acid comprises incubating the beads with an NaOH solution and washing the beads with wash buffer. In some embodiments, the washing is performed with 2 or more rounds of wash buffer.

In some embodiments, the NaOH used for treatment of beads comprising cellulose is 1M (equivalent to 1N) or less.

In some embodiments, treated cellulose-coated beads have favorable characteristics for nucleic acid binding and elution, allowing both strong nucleic acid binding and elution that will not damage the nucleic acid. For example, the yield of eluted nucleic acid from cellulose-coated beads may be similar over a range of pH of the binding buffer from pH 5-9.

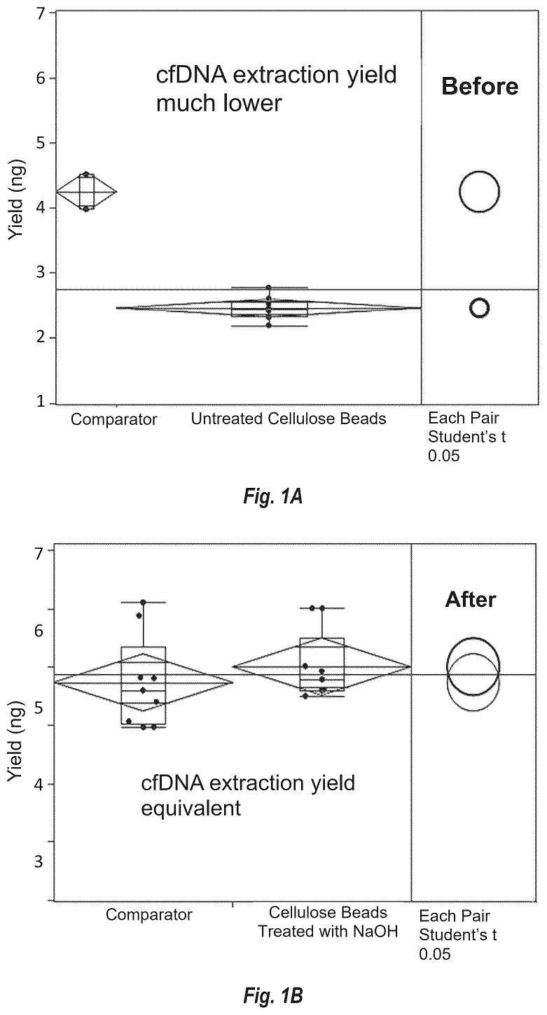

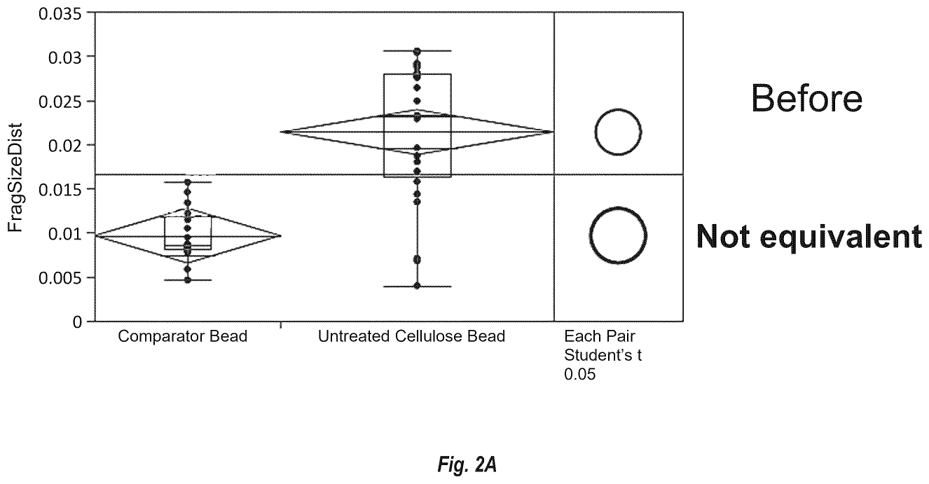

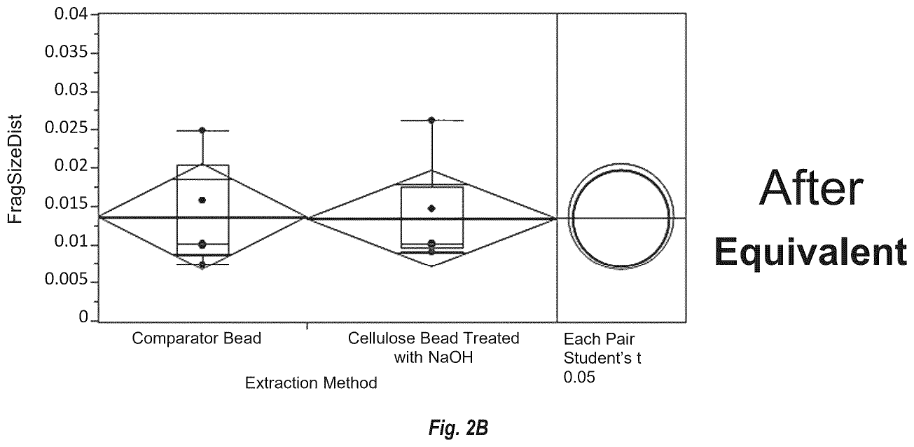

The effects of NaOH treatment on cellulose-coated beads are shown in FIGS. 1B and 2B, which show the similarity of treated cellulose-coated beads to comparator beads (Bioo Scientific beads comprised in NextPrep-Mag Kit 3825-01) for yield and fragment size of cfDNA extraction. In contrast, FIGS. 1A and 2A show the lower yield and larger fragment size of cfDNA extraction, in relation to comparator beads, for untreated cellulose-coated beads.

### C. NaOH Concentration and Treatment Times

In some embodiments, a relatively low concentration of NaOH may be used to treat beads. In some embodiments, concentration of less than 1.5M avoid unwanted dissolution of cellulose and reduction in the ability of treated beads to bind to nucleic acids.

In some embodiments, the NaOH solution comprises 0.2 to 1.0M NaOH. In some embodiments, the NaOH solution comprises 0.3 to 0.8M NaOH. In some embodiments, the NaOH solution comprises 0.5M NaOH.

In some embodiments, the incubating step is at least 60 minutes. In some embodiments, the incubating step is from 6 to 48 hours. In some embodiments, the incubating step is from 8 to 24 hours. In some embodiments, the incubating step is from 1 to 12 hours. In some embodiments, the NaOH solution comprises 0.5M NaOH and the incubating step is 8 hours. In some embodiments, the incubating step is 1, 2, 3, 4, 5, 6, 7, 8, 9, 10, 11, 12, 13, 14, 15, 16, 17, 18, 24, 36, 42, or 48 hours.

In some embodiments, the treatment with NaOH is treatment with 0.2 to 1.0M NaOH for 6 to 48 hours. In some embodiments, the treatment with NaOH is treatment with 0.3 to 0.8M NaOH for 6 to 24 hours. In some embodiments, the treatment with NaOH is treatment with 0.5M NaOH for 6 to 8 hours.

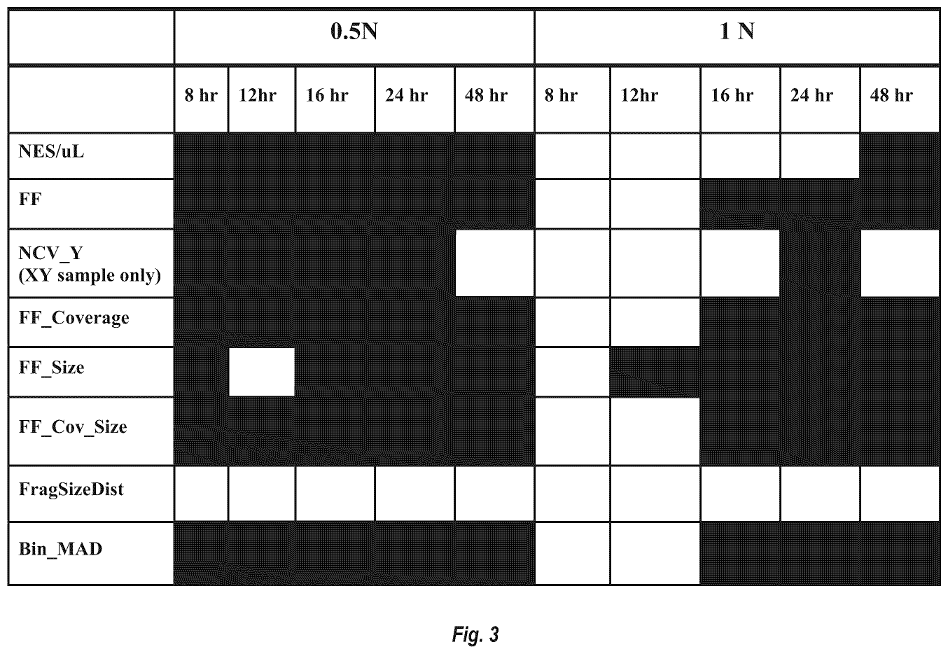

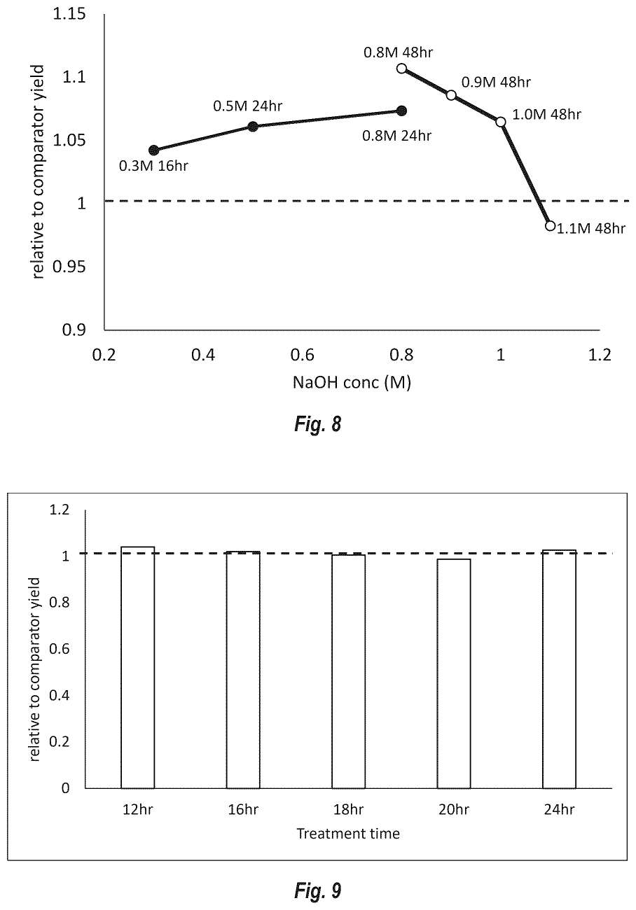

FIGS. 3-7 and 9 show results on experiments with different treatment conditions, and FIG. 8 highlights that relatively small changes in time or NaOH concentration could impact the yield of cfDNA extraction in relation to comparator beads. Since downstream assays may be dependent upon similarities with presently used extraction methods, the ability to “tune” the NaOH treatment to maintain similarity to comparators is important, as described below.

### D. Effect of Treatment on Binding Area and Binding Capacity

In some embodiments, NaOH treatment may change the structural characteristics of the cellulose-coated beads. In some embodiments, the binding area of the cellulose coating increases after treating with NaOH. If the binding area of the cellulose is increased, this allows for more surface area to bind nucleic acids and can increase the binding capacity of the beads.

In some embodiments, the binding capacity of the beads increases after treating with NaOH. In some embodiments, the binding capacity of the treated cellulose-coated beads for DNA is 1.5-2.5 times higher when compared to untreated cellulose-coated beads, optionally such as 1.5, 1.6, 1.7, 1.8, 1.9. 2, 2.1, 2.2, 2.3, 2.4, or 2.5 times higher and any range assembled from any of these numbers. In some embodiments, the binding capacity of the treated cellulose-coated beads is 290-330 ng/μL. In some embodiments, the binding capacity is measured with treated cellulose-coated beads in a solution of 113-138 mg of beads/mL.

In some embodiments, the increased binding capacity of the treated cellulose-coated beads is irreversible. In other words, the change to the cellulose-coated bead induced by the NaOH treatment may be permanent. As such, the treated cellulose-coated beads can be stored and retain their characteristics. Such stability of treated cellulose-coated beads can allow for preparation of kits described herein.

II. Methods of Extraction of cfDNA Using Treated Cellulose-Coated Beads

In some embodiments, treated cellulose-coated beads are used in methods of nucleic acid extraction. In such methods, treated cellulose-coated beads may be used to bind cfDNA from a sample, and then the bound cfDNA may be eluted.

In some embodiments, the characteristics of cfDNA binding of the treated cellulose-coated beads allows for tuning of the extraction to achieve results similar to comparator beads.

Representative conditions for preparing treated beads that allow for successfully extracting cfDNA are shown in FIGS. 1-9.

In some embodiments, a method of extracting cfDNA from a sample comprises combining (i) the sample; (ii) a proteinase; and (iii) treated cellulose-coated beads, wherein the cellulose-coated beads are prepared by a treatment with NaOH and binding the cfDNA to the cellulose-coated beads. As will be described herein, the composition of a binding buffer for the binding step may improve the efficiency of extracting cfDNA.

In some embodiments, the method comprises allowing binding of the cfDNA to the beads after the combining and washing the bound cfDNA with a wash buffer. In some embodiments, the method comprises eluting the bound cfDNA with a resuspension buffer after the washing. In some embodiments, the method further comprises washing the bound cfDNA with an ethanol wash after the washing step and before the eluting step. In other words, an ethanol wash may be performed after the washing step with a wash buffer and before the eluting step.

The sample may be any sample that comprises cfDNA. In some embodiments, the sample is from a human patient. In some embodiments, the sample comprises cfDNA of interest, such as fetal cfDNA in a sample from a pregnant patient. In some embodiments, the sample is blood. In some embodiments, the sample is plasma.

Proteinase treatment is a common step in isolation of nucleic acid, such as extraction from blood or plasma. For example, cfDNA may be associated with histones that interfere with cfDNA isolation, and a proteinase may serve to remove the histones. In some embodiments, a proteinase is comprised in the binding buffer and the sample is applied to the binding buffer. In some embodiments, a proteinase is combined with the sample before the treated cellulose-coated beads are combined with the sample. In some embodiments, the proteinase is proteinase K.

In some embodiments, the method further comprises treating cellulose-coated beads with NaOH before the combining. The treating may be a treatment as described herein, such as with a relatively low concentration of NaOH.

a. Cell-Free DNA and Diagnostics with Cell-Free DNA

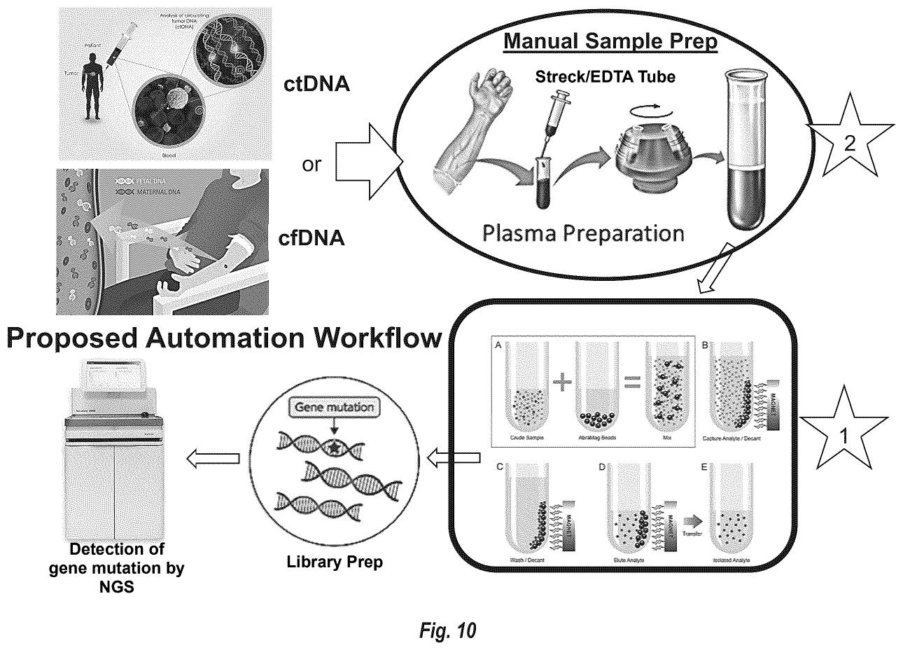

FIG. 10 provides an overview of cfDNA extraction using magnetic beads and how it might be used in a diagnostic assay to assess for gene mutations using plasma from a patient by sequencing. In some embodiments, the cfDNA is comprised in blood or plasma. In some embodiments, a sample comprising cfDNA is added to treated magnetic beads (as described herein) and mixed. In some embodiments, washes (such as with wash buffer and then ethanol) can be performed using a magnet to collect beads separate from the supernatant (such as with a magnetic stand or plate magnet). In some embodiments, after washes, the “isolated analyte” can be eluted, which would comprise cfDNA.

In some embodiments, the cfDNA comprises fetal cfDNA and/or circulating tumor DNA.

In some embodiments, the cfDNA is circulating tumor DNA (ctDNA) from blood or plasma. Such measurements of ctDNA from blood or plasma may be termed “a liquid biopsy.” Analysis of ctDNA provides a range of diagnostic information, such as measuring tumor heterogeneity, determining treatment efficacy, assessing remission or progression, or initial screening for presence of disease.

In some embodiments, the cfDNA of interest comprises fetal cfDNA from the blood or plasma of a pregnant woman. Uses of fetal cfDNA are well-known for non-invasive prenatal testing (NIPT), which is early genetic screening (as early as 10 weeks into a pregnancy) for chromosomal conditions using just one tube of blood from a pregnant mother. NIPT provides high detection rates, low false-positive results, and little risk to mother and baby.

### B. Tunable Extraction

Since numerous clinical diagnostics are based on cfDNA analysis, a user may wish for a new method of cfDNA extraction to parallel an existing method.

In some embodiments, the fragment size bound (and released) by treated cellulose-coated beads can be modified by varying the NaOH treatment (such as the time of treatment or the concentration of NaOH). In this way, a user can tune a method of cfDNA extraction by using treated beads that bind the desired ranges of fragment sizes and produce the desired yield relative to currently used extraction methods.

In some embodiments, the treatment with NaOH is selected to produce a desired extraction efficiency and/or a desired fragment size of cfDNA.

FIGS. 1-9 show data on how one skilled in the art could treat beads to have desired properties. For example, as shown in FIG. 8, the relative yield of cfDNA extraction in relation to that of comparator beads could be tuned by different concentrations of NaOH and different time-periods of treatment.

In some embodiments, the desired extraction efficiency is 85% or more, 90% or more, or 95% or more of the amount of cfDNA comprised in the sample.

In some embodiments, the desired fragment size is 50-500 base pairs. In some embodiments, the desired fragment size is 140-180 base pairs.

C. Extraction of cfDNA from Plasma

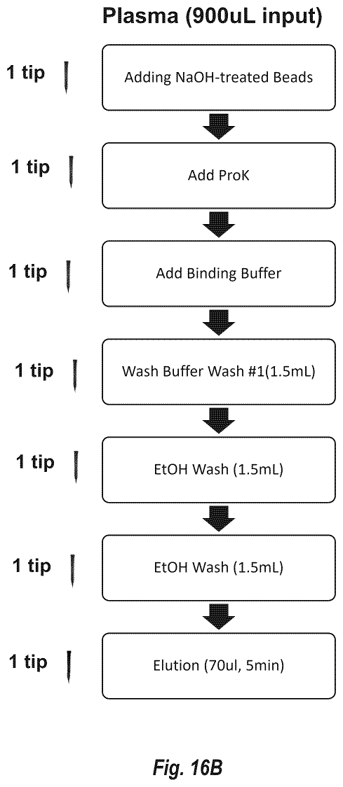

FIG. 10 shows a representative method of cfDNA extraction and analysis that includes preparation of plasma from patient's blood, and such standard methods of cfDNA analysis include a step of plasma preparation. Representative steps of a method for extracting DNA from plasma after plasma preparation are shown in FIG. 16B.

Plasma preparation includes adding patient blood to a Streck tube (or other tube with EDTA), spinning the sample in a centrifuge, and collecting the plasma that has been separated from the blood. For examples, barcoded samples may be centrifuged at 1600×g for 10 minutes at 4° C. with the lowest brake setting. When the centrifuge comes to a complete stop, the sample tubes are removed and plasma isolation should begin within 15 minutes. If more than 15 minutes elapse before plasma isolation, the samples should be centrifuged again. Thus, plasma isolation often requires two centrifugations, a first slow centrifugation to separate plasma and a second fast to remove residual cells and cell debris. If a user is running in a 96-well format, a centrifuge capable of holding 96 samples or multiple centrifuges are required in order to start the plasma isolation within 15 minutes.

The step of preparing plasma cannot be automated, as it requires centrifugation and pipetting off of the plasma from the blood after the centrifugation. The manual step of preparing plasma from blood before beginning cfDNA thus can be a roadblock in attempts to simplify and automate cfDNA extraction protocols. While plasma can be used to extract cfDNA (and is generally the starting material for present commercial protocols), methods that avoid a requirement to prepare plasma could simplify workflows and allow for greater automation.

In some embodiments, a method of cfDNA from plasma can be automated. Further, as described herein, normalization of extracted cfDNA, library preparation, and library normalization can also be automated allowing for an automated workflow from plasma to sequencing results.

D. Extraction of cfDNA Directly from Blood

In some embodiments, the method does not require preparation of plasma from blood before combining the sample with the proteinase.

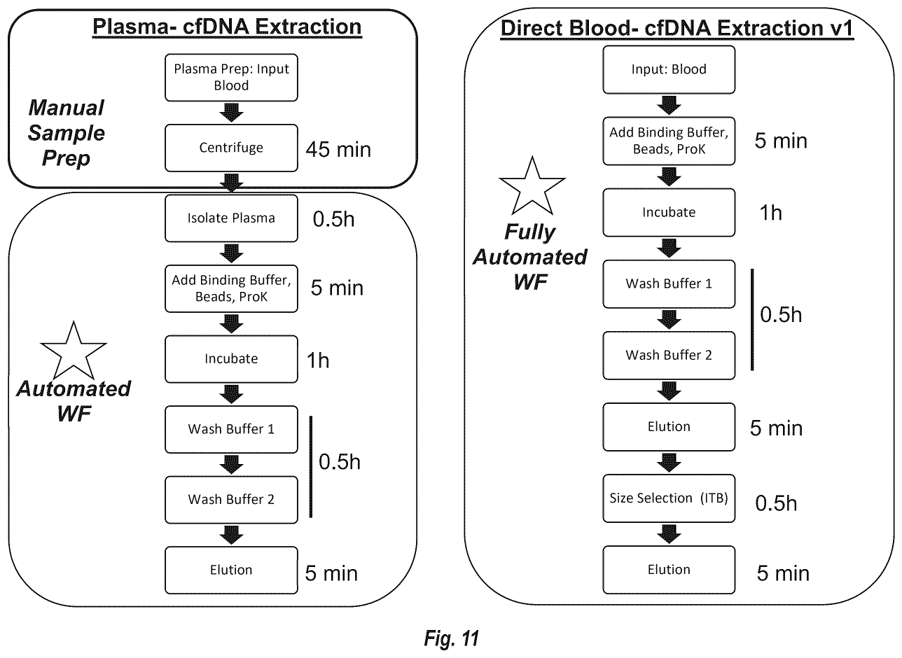

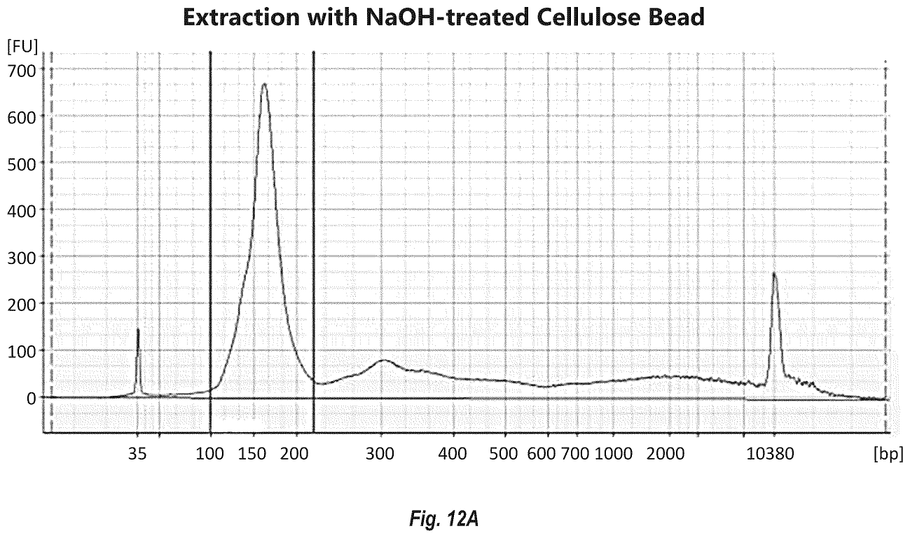

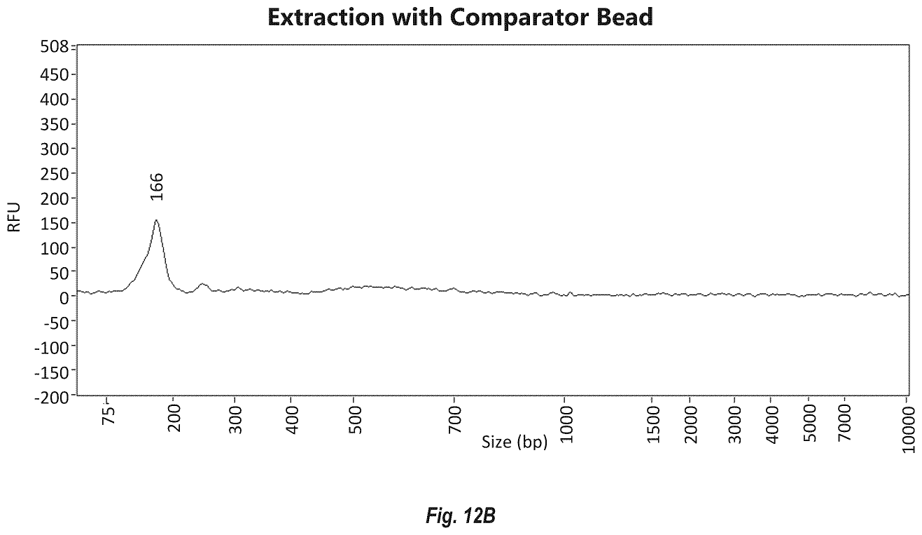

In some embodiments, cfDNA is extracted from blood without preparing plasma from the blood. As used herein, extracting cfDNA “directly from blood” means that plasma was not prepared from the blood before extracting cfDNA. As shown in FIG. 10, current methods of cfDNA preparation generally include a step of preparing plasma from blood before extracting cfDNA. FIGS. 11, 15A, 15B, and 16A present some exemplary protocols that may be used to extraction cfDNA directly from blood, and FIG. 12-14 show data on successful DNA extraction from blood.

### E. Size Selection for Removing Genomic DNA

When cfDNA is extracted directly from blood, there may be inherent risk of extracting significant amounts of genomic DNA as well. This is because beads for extraction of cfDNA would also be capable of binding genomic DNA. Without a step of preparing plasma from blood before beginning extraction, genomic DNA from the nuclei of blood cells could therefore potentially contaminate cfDNA extractions made directly from blood. For example, if a user wants to sequence fetal cfDNA from a mother's blood sample, the user would not want maternal genomic DNA to overwhelm the sequencing results.

cfDNA generally comprises relatively small fragments. For example Shi et al., Theranostics 10 (11): 4737-4748 (2020) have described that a dominant peak of cfDNA fragment size across different population is often near 166 base pairs. In contrast, genomic DNA would be expected to be a larger size. Therefore, size selection to enrich cfDNA may improve results for cfDNA extraction directly from blood.

In some embodiments, size selection of an initial extraction of nucleic acid is performed to increase the percentage of cfDNA in the extracted nucleic acid. In some embodiments, the size selection is performed using carboxylate beads. Such carboxylate beads may be Solid Phase Reversible Immobilization beads (SPRI beads, Beckman Coulter) or Illumina Tune Beads (ITB). In some embodiments, the carboxylate beads comprise a polystyrene core surrounded by a layer of magnetite, which is coated with carboxyl molecules. In some embodiments, the carboxylate beads are paramagnetic (i.e., magnetic only in a magnetic field).

In some embodiments, a method comprises, after eluting bound cfDNA from treated cellulose-coated beads, binding the eluted cfDNA to carboxylate beads and performing size selection, and eluting the bound cfDNA from the carboxylate beads with a resuspension buffer.

In some embodiments, performing size selection comprises removing genomic DNA from the sample and retaining cfDNA. In this way, the eluted nucleic acids after size selection with carboxylate beads are enriched for cfDNA.

In some embodiments, carboxylate beads in different concentrations of PEG are used for size selection. In some embodiments, performing size selection comprises two rounds of binding to carboxylate beads, wherein the first round of binding is performed with buffer having a higher concentration of PEG and the second round of binding is performed with a buffer having a lower concentration of PEG. In other words, the higher concentration of PEG in the buffer for the first round of binding for size selection is relative to the concentration of PEG in the buffer for the second round of binding, and the lower concentration of PEG in the buffer for the second round of binding for size selection is relative to the concentration of PEG in the buffer for the first round of binding. In some embodiments, carboxylate beads in the buffer with a higher concentration of PEG preferentially bind genomic DNA in the sample, and the genomic DNA is removed from the sample. In some embodiments, carboxylate beads in the buffer with a lower concentration of PEG preferentially bind cfDNA.

In some embodiments, the carboxylate beads are Illumina Tune Beads, as described in, for example, Illumina COVIDSeq Test Reference Guide, Illumina, Document #1000000126053 v04 (2021). In some embodiments, the carboxylate beads are SPRI beads (Beckman Coulter).

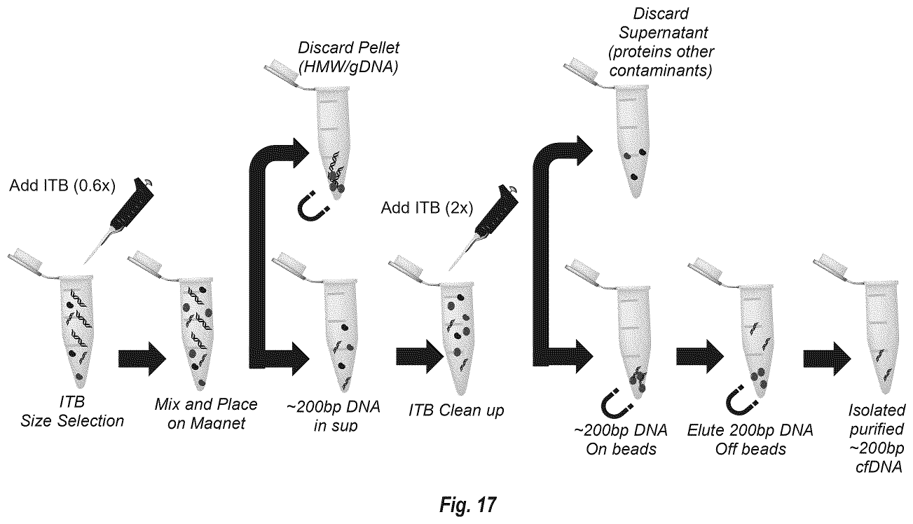

An exemplary method of size selection to remove high molecular weight DNA (HMW DNA) and genomic DNA (gDNA) with different concentrations of ITB is shown in FIG. 17.

### F. Binding Buffer

In some embodiments, the components of a binding buffer improve the binding of nucleic acid to the treated beads.

In some embodiments, the binding buffer comprises a chaotropic agent. The chaotropic agent may work to improve nucleic acid binding and disrupt hydrogen bonds to allow for washing away of contaminants. In some embodiments, the chaotropic agent is guanidinium thiocyanate (GuSCN). In some embodiments, the concentration of GuSCN is less than or equal to 7M. In some embodiments, the concentration of GuSCN is 2-5M.

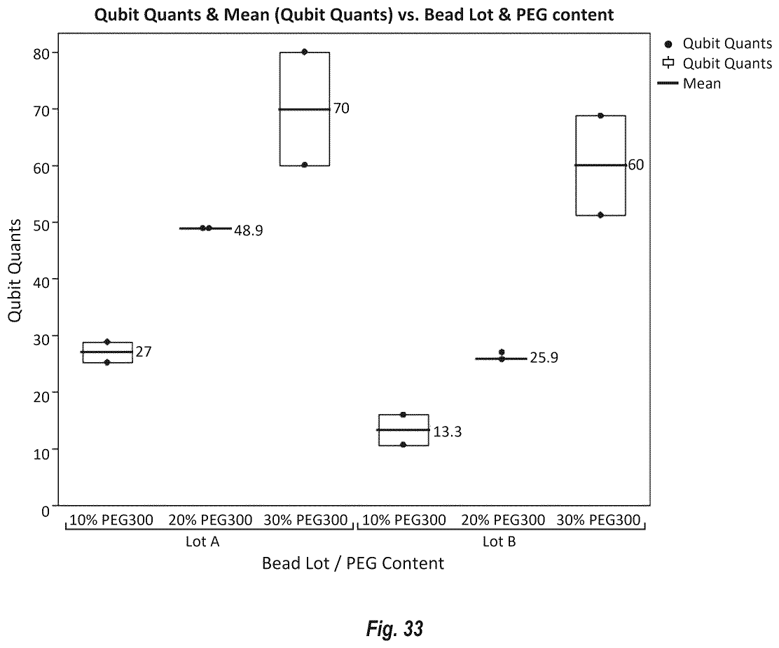

In some embodiments, the binding buffer comprises PEG, which may work to improve the rate of DNA hybridization. In some embodiments, the PEG comprises PEG200, PEG300, and/or PEG400. In some embodiments, a higher percentage of PEG (weight/volume) leads to a greater capture of DNA by cellulose-coated beads. In some embodiments, the PEG concentration of a binding buffer is 30%-40% (weight/volume). As shown for normalization methods described below, an increase in PEG300 concentration from 10% to 30% increases the capture of DNA by beads (see, for example, data in FIG. 33 on capture with untreated cellulose-coated beads for normalization methods).

As described in Example 2, use of lower-molecular weight PEG (as compared to, for example, PEG8000) in the binding buffer may help achieve a desired cfDNA extraction yield. In some embodiments, improved binding characteristics of DNA binding to cellulose-coated beads (whether for cfDNA extraction or library normalization) is seen with a binding buffer comprising PEG200, PEG300, and/or PEG400 as compared to a binding buffer comprising PEG3000, PEG4000, PEG5000, PEG6000, PEG7000, and/or PEG8000. Thus, by lower-molecular weight PEG it is meant PEG200, PEG300, and/or PEG400.

PEG 8000 is commonly used as an additive for bead-based DNA capture. For example, binding of DNA to carboxylated magnetic beads is often performed in a binding buffer comprising PEG8000 to precipitate fewer contaminants (see, for example, Mayjonade et al., Biotechniques 61 (4): 203-205 (2016)). Further, commercially available AMPure XP beads (Beckman) are generally used for purifying DNA fragments with a buffer containing PEG8000 (Reynoso et al. Plant Physiol. 176:270-281 (2018) including Appendix I). However, as described herein, users may desire to limit DNA capture using a binding buffer that comprises a lower-molecular wight PEG, such as PEG200, PEG300, and/or PEG400.

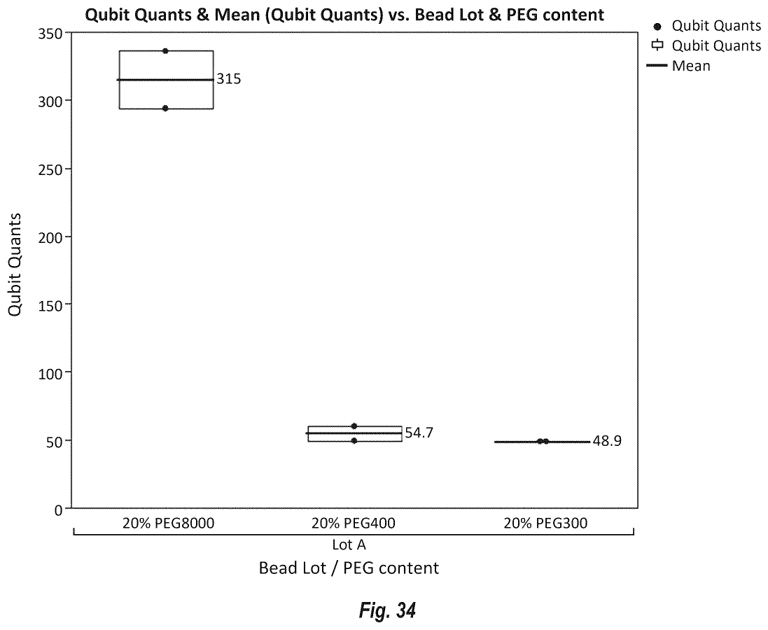

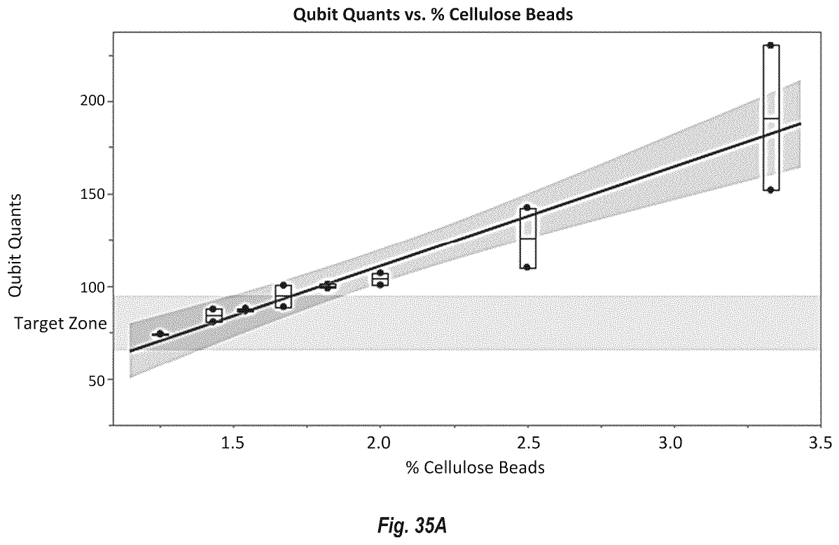

For normalization protocols described below, use of PEG8000 in binding buffer led to approximately 6-fold greater DNA capture as compared to PEG300 or PEG400, as shown in FIG. 34. For both cfDNA extraction and normalization, a user may wish to limit DNA capture (such as to achieve a desired concentration of extracted cfDNA or of normalized library) and accordingly will use a binding buffer comprising a lower-molecular weight PEG instead of PEG8000 or other higher-molecular weight PEG.

In some embodiments, the binding buffer comprises a buffering agent, such as Tris. In some embodiments, the Tris concentration is 15-25 mM. In some embodiments, the Tris is pH 6.5 to pH 7.

In some embodiments, the binding buffer comprises a detergent. In some embodiments, a detergent in the binding buffer promotes lysis and degradation of lipid bilayers. In some embodiments, the detergent is Tween-20. In some embodiments, the Tween-20 concentration is 0.1%-0.2% (weight/volume).

In some embodiments, the binding buffer comprises PEG and GuSCN. In some embodiments, the binding buffer comprises 30%-40% (weight/volume) of PEG200, PEG300, and/or PEG400 and 2-5M GuSCN.

In some embodiments, the binding buffer comprises PEG, Tris, GuSCN, and Tween-20. In some embodiments, the binding buffer comprises (a) 30%-40% (weight/volume) of PEG200, PEG300, and/or PEG400; (b) 15-25 mM Tris pH 6.5 to pH 7; (c) 2-5M GuSCN; and (d) 0.1%-0.2% (weight/volume) Tween-20.

In some embodiments, the binding buffer has a viscosity of 8.0-8.5 mPa*s at 25° C. Proper viscosity of the binding buffer may improve mixing and binding of nucleic acids to the treated beads.

In some embodiments, the binding buffer has a conductivity of 49-51 ms/cm at 25° C. Proper conductivity of the binding buffer can improve electrostatic interactions between beads and nucleic acids.

In some embodiments, the binding buffer has a refractive index of 70%-80% Brix at 25° C.

### G. Wash Buffers

In some embodiments, the characteristics of the wash buffer may improve the ability of nucleic acid to remove bound to treated beads during washing and/or improve the ability to wash away contaminants.

In some embodiments, the wash buffer comprises a chaotropic agent. In some embodiments, the chaotropic agent is GuSCN. In some embodiments, the concentration of GuSCN is less than 3.5M. In some embodiments, the concentration of GuSCN is 1-3M.

In some embodiments, the wash buffer comprises PEG. In some embodiments, the PEG comprises PEG200, PEG300, and/or PEG400. In some embodiments, the PEG concentration is 15%-20% (weight/volume).

In some embodiments, the wash buffer comprises Tris and/or a detergent. In some embodiments, the Tris concentration is 8-12 mM. In some embodiments, the Tris is pH 6.5 to pH 7. In some embodiments, the detergent is Tween-20. In some embodiments, the Tween-20 concentration is 0.05%-0.1% (weight/volume).

In some embodiments, the wash buffer comprises PEG and GuSCN. In some embodiments, the wash buffer comprises 15%-20% (weight/volume) PEG200, PEG300, and/or PEG400 and 1-3M GuSCN.

In some embodiments, the wash buffer comprises PEG, Tris, GuSCN, and Tween-20. In some embodiments, the wash buffer comprises (a) 15%-20% (weight/volume) PEG200, PEG300, and/or PEG400; (b) 8-12 mM Tris; (c) 1-3M GuSCN; and (d) 0.05%-0.1% (weight/volume) Tween-20.

In some embodiments, the wash buffer has a viscosity of 1.8-2.0 mPa*s at 25° C. In some embodiments, the wash buffer has a conductivity of 85-90 ms/cm at 25° C. In some embodiments, wherein the wash buffer has a refractive index of 40-45% Brix at 25° C.

### III. Methods of Normalization Using Cellulose-Coated Beads

The presently described cellulose-coated beads may be used for normalization, such as normalization of a library after its preparation. “Normalization” or “library normalization,” as used herein, refers to the process of diluting libraries of variable concentration to the same or a similar concentration. A user may wish for all samples to have similar amount of DNA as starting materials (see, for example, Userguide for Normalization of DNA/RNA Samples using the DNA/RNA Normalization Calculator, Eppendorff, epMotion No. 015). Normalization may occur either before or after amplification.

In some embodiments, a normalization is performed after DNA extraction, such as ctDNA extraction from plasma. This step may be performed immediately after extraction. A normalization step after DNA extraction promotes successful library generation by normalizing a wide concentration range of ctDNA to a narrower range. Without such a normalization step, an automation workflow may have to pause to allow for manual ctDNA quantification and manual concentration adjustment in each well. Such manual steps are automation-unfriendly and slow down the overall time for generating data from extracted ctDNA.

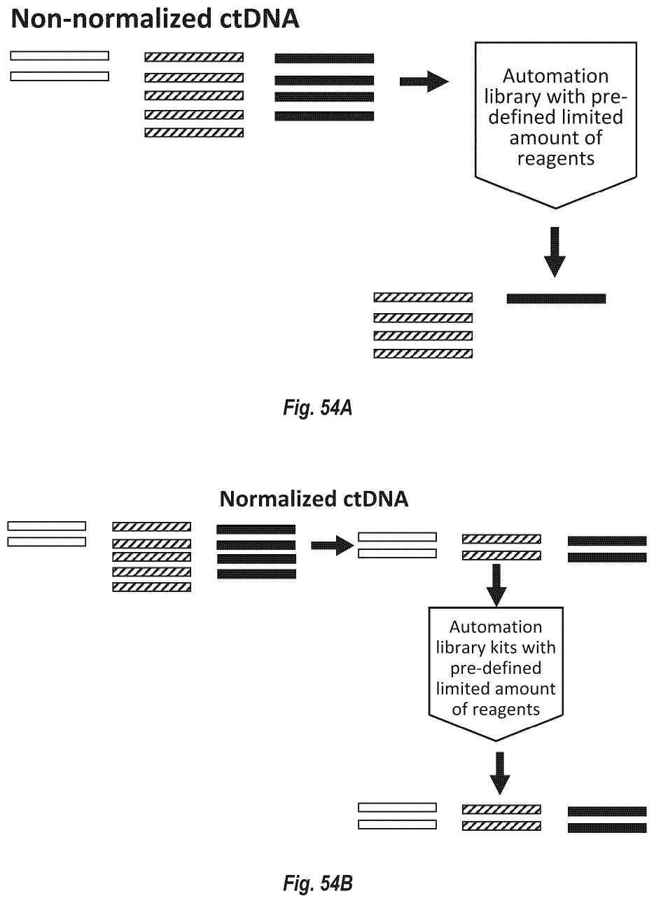

FIGS. 54A and 54B outline the advantages of normalization of ctDNA before library preparation. As shown in FIG. 54A, if ctDNA is not normalized before library preparation, there can be a range of concentrations of ctDNA samples, some with high concentrations and some with low concentration. However, the pre-defined amount of reagents in automation library kits may not be proper for samples with either low or high concentrations. For example, the library kit reagents may be insufficient for ctDNA samples with higher concentrations, and lower concentration ctDNA samples may be lost in processing. In contrast, FIG. 54B shows that library conversion can have a higher success rate if ctDNA is normalized before library preparation, such that a greater percentages of ctDNAs can be successfully converted into libraries.

In some embodiments, a normalization step is performed after DNA extraction and before library preparation.

In addition, a normalization step is often performed immediately prior to loading samples for sequencing. In some embodiments, normalization is performed after a PCR amplification step and before sequencing the normalized library. In some embodiments, a normalization step just before sequencing ensures that all library samples have similar concentrations that allow for successful sequencing runs.

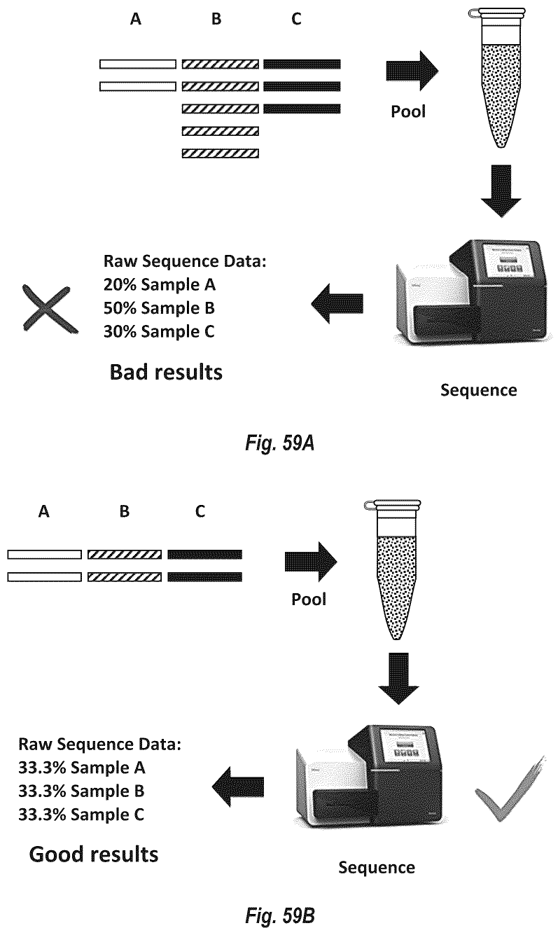

FIGS. 59A and 59B shows the advantages of normalizing libraries before sequencing. As shown in FIG. 59A, non-normalized libraries will have different concentrations. Thus, when libraries are pooled for sequencing, there will be a wide range of results for different libraries. For example, Sample B in FIG. 59A would account for 50% of the raw sequence data, as it had a greater number of libraries before the pooling without any normalization step. In contrast, FIG. 59B shows the effect of normalizing libraries before pooling and sequencing. This normalization allows for the different library samples (i.e., Samples A, B, and C) to have similar representation in the sequencing results. In this way, normalization of libraries before sequencing allows for more robust analysis of sequencing data by reducing bias from high-concentration libraries.

In some embodiments, the nucleic acid library is amplified before combining the library with cellulose-coated beads. In some embodiments, amplification before normalization is performed for libraries comprising large fragments.

In some embodiments, normalization helps to ensure an even read distribution for all samples during sequencing. In other words, normalizing libraries can help to ensure even representation in the final sequencing data.

In some embodiments, the normalization uses cellulose-coated beads. In some embodiments, the cellulose-coated beads are treated with NaOH (as described above for cfDNA extraction). In some embodiments, the cellulose-coated beads are untreated. The cellulose-coated beads may be used with binding and wash buffers as described for cfDNA extraction. In some embodiments, the large size of the present beads (1 μm to 10 μm) also helps for a user to visualize the small number of beads that may be used for normalization.

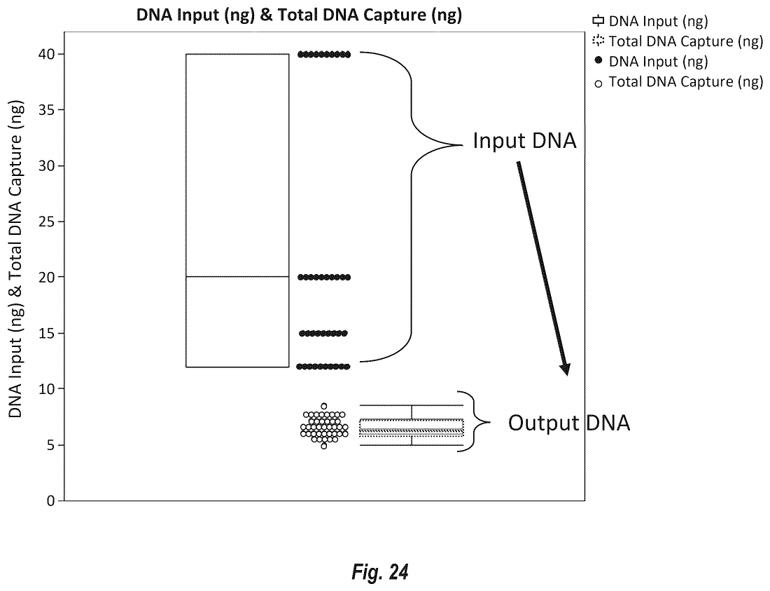

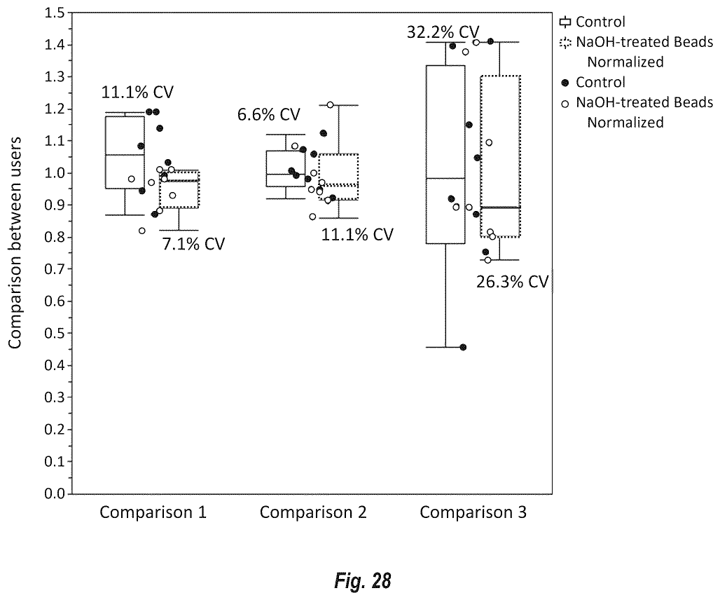

In some embodiments, normalizing a library improves the quality of downstream results (such as sequencing). For example, a normalized library may have a lower coefficient of variance (CV) compared to the library before the method of normalizing, as shown in FIG. 24. FIG. 28 shows that the change in CV with normalization was similar to that for manual normalization across users.

In some embodiments, a method of normalizing a nucleic acid library comprises the steps of combining the library with cellulose-coated beads prepared by treatment with NaOH and allowing binding of the nucleic acid to the beads in a binding buffer. Then, the method may comprise washing the bound nucleic acid with one or more rounds of a wash buffer and eluting the bound nucleic acid with a resuspension buffer. In some embodiments, the eluted nucleic acid comprises a normalized library.

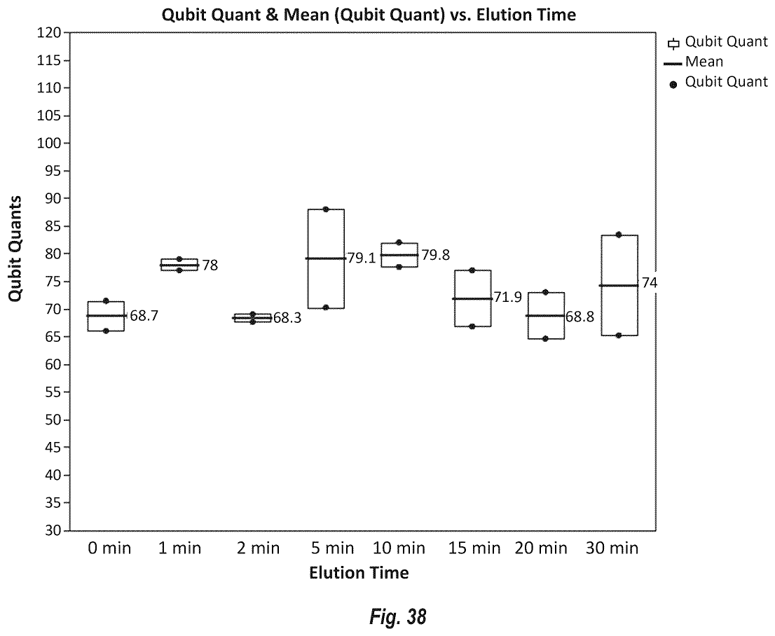

In some embodiments, the method further comprises washing the bound nucleic acid with ethanol wash after the washing and before the eluting step. As shown in FIG. 38 for untreated cellulose-coated beads, elution of the bound normalized library may proceed rapidly with an incubation of 1-5 minutes with a resuspension buffer (RSB, which may be a pH 8.5, Tris buffered solution, with 10-100 mM NaCl) being sufficient.

In some embodiments, the normalization is performed with treated cellulose-coated beads. In some embodiments, the normalization is performed with untreated cellulose-coated beads.

In some embodiments, the method further comprises treating cellulose-coated beads with NaOH before the combining. Exemplary methods of treating cellulose-coated beads with NaOH are described herein.

In some embodiments, the method uses untreated cellulose-coated beads. As used herein, “untreated cellulose-coated beads” or “non-treated beads” refers to cellulose-coated beads that are not subjected to a treatment that increases their binding capacity (i.e., untreated beads are not treated with NaOH). However, untreated beads may be subject to washing with a wash buffer or incubation with another agent that does not impact the binding capacity of the cellulose-coated beads.

While cellulose is known to bind to nucleic acids, the specific conditions of the present normalization protocol may improve results with these beads (whether treated or untreated) in normalization. For example, a binding buffer with PEG400 improved results compared to a binding buffer comprising PEG8000 (data not shown, similar to the effect described for binding buffer for cfDNA extraction in Example 2).

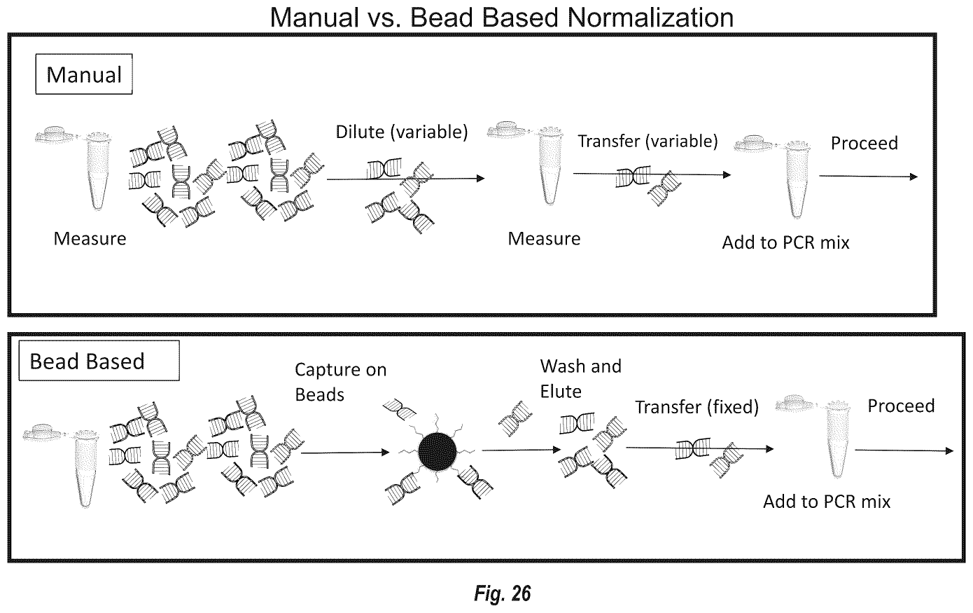

As shown in FIG. 26, an advantage of bead-based normalization is that it uses capture on beads to normalize, as opposed to manual normalization methods that require measure of library concentration and dilutions. The requirements for manually normalizing library concentrations are well-known in the art (see, for example Best Practices for Manually Normalizing Library Concentrations, Illumina, Apr. 22, 2021 and Userguide for Normalization of DNA/RNA Samples using the DNA/RNA Normalization Calculator, Eppendorff, epMotion No. 015). In some embodiments, a method of normalizing does not require calculation of the library concentration. In this way, a user may avoid time-consuming and cumbersome calculations and dilutions during normalization.

To manually normalize cfDNA (such as ctDNA), the user must stop the automation workflow and then manually sample, quantify, and dilute the cfDNA in the each well. Thus, manual normalization is time and user intensive (taking approximately 1-hour for an experienced user), though the manual process may reduce yield loss. In contrast, a bead-based normalization (BBN) does not require touchpoints by the user, allowing a short turnaround time (TAT) and a fully automated end-to-end (ETE) workflow. With a fully automated ETE workflow, the user can put plasma samples onto a robot, walk away, and return 4-5 hours later, and the entire process has automatically finished with the final library samples ready for sequencing. Such an fully automated ETE workflow thus eliminates human hand touch points during the workflow.

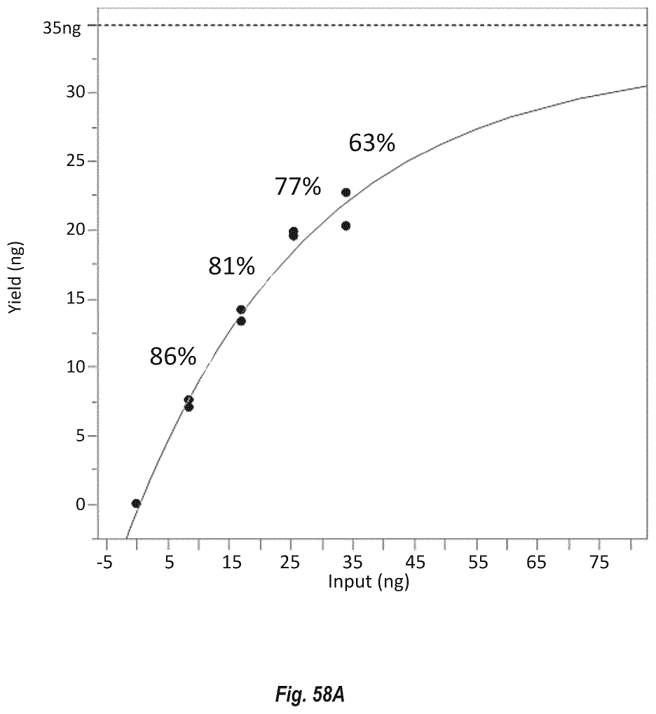

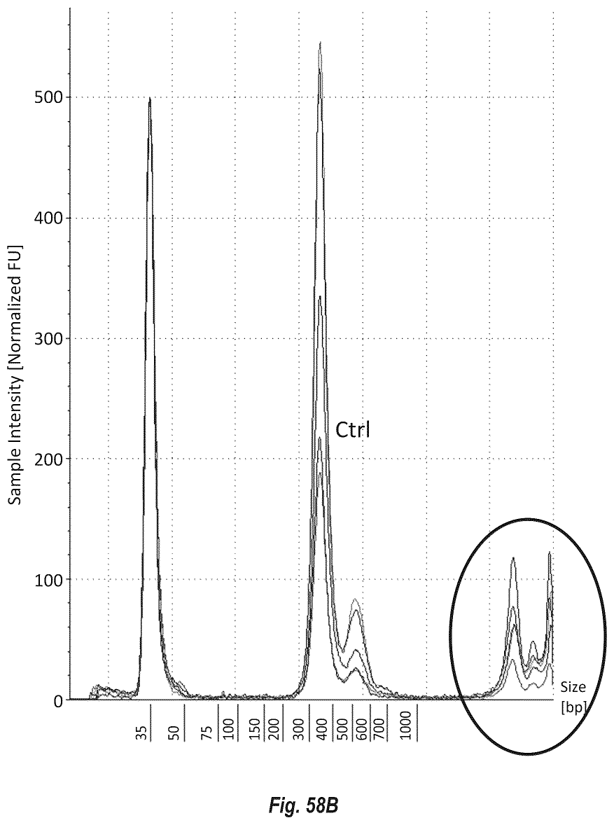

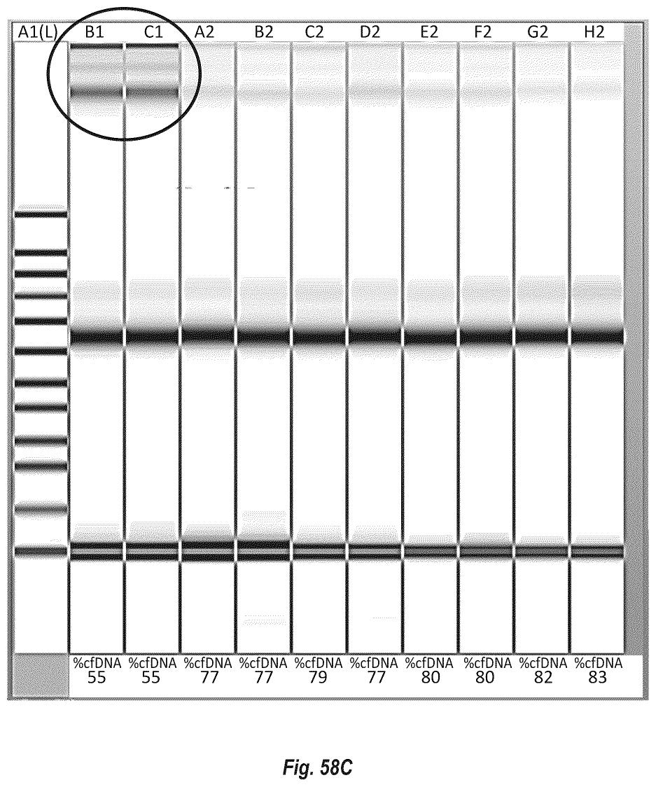

BBN with the presently described cellulose-coated beads also minimizes yield loss, such that yield is generally sufficient for sequencing. Example 12 provides details on an automated BBN protocol with the presently described cellulose-coated beads, with representative data presented in FIGS. 58A-58C. Such an automated BBN process can reduce time and effort in normalizing extracted cfDNA.

In some embodiments, a method of normalizing can be performed with end-to-end automation. Such ability to use automation can decrease the amount of additional time needed for normalization of a larger number of samples, because standard automated equipment may be used.

While some bead-linked transposome (BLT) methodologies are known to allow for bead-based normalization, such as Nextera™ XT and Illumina® DNA Prep, (M) Tagmentation (formerly known as Nextera DNA Flex), these methods require that the library be prepared by fragmentation by transposomes (e.g., tagmentation to incorporate adapters for amplification and/or sequencing of the library). However, a user may wish to avoid tagmentation using bead-linked transposomes for some library preparations. For example, a user may wish to avoid addition of adapter sequences, covalent binding of the DNA, and denaturation or PCR that may be required in methods using bead-linked transposomes.

Thus, in some embodiments the present beads are not linked to transposomes. Use of bead-based normalization protocols with cellulose-coated beads may be less expensive than normalization with bead-linked transposomes. In some embodiments, cellulose-coated beads may be diluted many folds prior to use for normalization methods, allowing cost savings. In some embodiments, cell-coated beads are also less expensive than other DNA-binding beads, such as streptavidin beads.

In some embodiments, methods with treated cellulose-coated beads allows for preparation of libraries with lower CV values in comparison to other bead-based methods.

As described herein, an advantage of the present bead-based methods for normalizing libraries with treated magnetic cellulose-coated beads is that they can be used with libraries generated by a variety of different means, such as library preparation with ligation of adapters (such as representative library preparation described for VeriSeq NIPT as described in VeriSeq NIPT Solution v2 Package Insert, Illumina, Document #1000000078751v06, August 2021). Further, the present method is compatible with different steps of library preparation, such as normalizing library preparation intermediates and/or normalizing library preparation end products.

In some embodiments, no modifications to library fragments are needed for normalizing. For example, library intermediates can be normalized before the addition of adapters (such as by ligation) that is standard in many library preparation protocols.

In some embodiments, the present beads allow for normalization of libraries with low DNA concentrations (such as pg/μL levels), while other normalization protocols may have difficulty when used with libraries with such low concentrations. In some embodiments, a method of normalization with cellulose-coated beads is flexible in determining targeted concentration and/or has better linearity for normalization yield over a range of concentrations of library products.

In one example, extracted cfDNA may already comprise relatively small fragments, and the user may not want to further fragment the cfDNA by using tagmentation with BLTs to prepare the library. In such an example, a user may utilize well-known techniques to ligate appropriate adapters to the ends of cfDNA fragments and then normalize the library with a bead-based approach described herein. In other words, the present methods can allow a user to utilize the advantages of bead-based normalization, without a requirement that the user prepare the library with BLTs.

In another example, the present methods of bead-based normalization may be used with long-read sequencing technologies or other uses wherein a user wants to maintain long library fragments within the library. For example, the desired library fragments may comprise 6000-8000 base pairs each. Long-read sequencing of such large fragments can be used for sequencing stretches of highly repetitive elements and for generating long reads for de novo assembly, and library normalization with BLTs may be undesired in this method as it would lead to further fragmentation of long library fragments. In other words, the present cellulose-coated beads can allow for bead-based normalization while retaining large library fragments.

In some embodiments, the present methods of normalization using cellulose-coated beads can also allow for certain advantages of bead-based normalization over manual normalization (such as ease of use and shorter protocol time), without requiring use of BLTs in library preparation. An overview of bead-based normalization as presently described and standard manual normalization is presented in FIG. 26. In some embodiments, the present methods of bead-based normalization also avoid requirements of manual normalization for specific equipment to evaluate libraries. Exemplary cost saving of bead-based normalization to avoid additional equipment and kits is summarized, for example, in Best Practices for Standard and Bead-Based Normalization in Nextera® XT DNA Library Preparation Kits, Illumina, Pub. No. 470-2016-007-B, 2017.

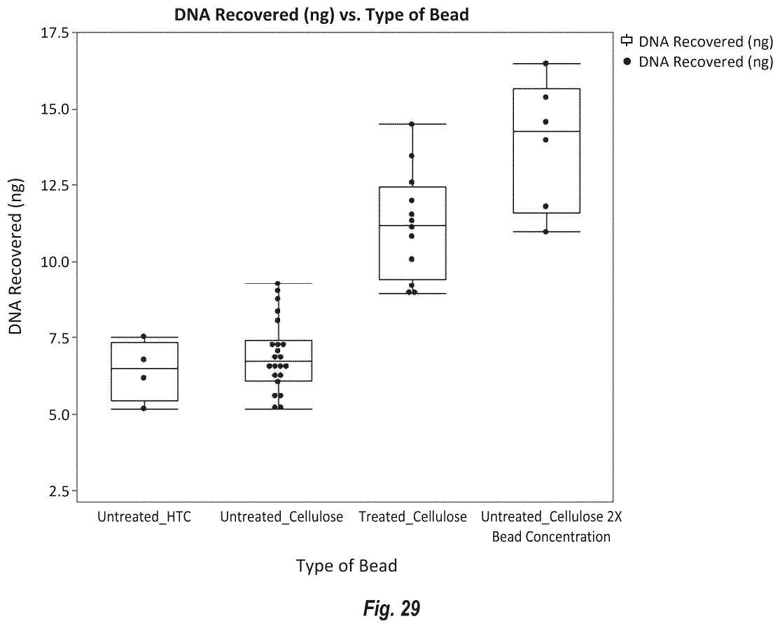

In some embodiments, NaOH-treated cellulose-coated beads have 80-120% higher binding capacity and allow for higher yield from a normalization procedure (see, for example, the comparison in FIG. 29 between DNA recovery with untreated beads versus NaOH-treated beads). Such treated beads may be especially useful for methods where a user has a large amount of library input.

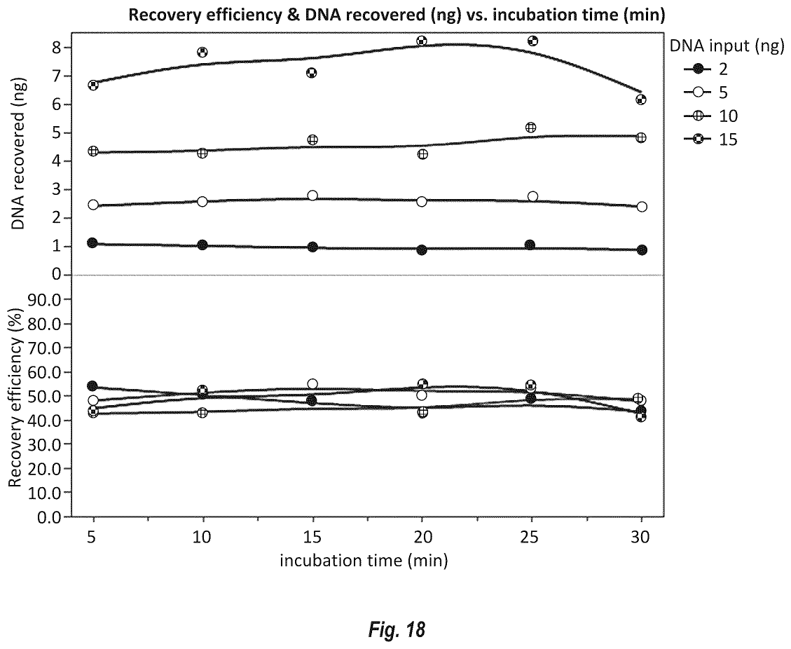

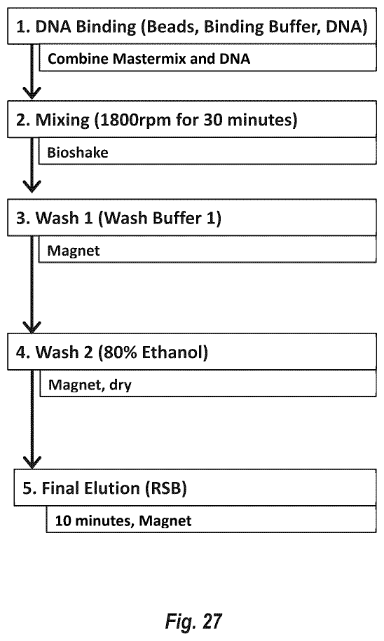

In some embodiments, NaOH-treated cellulose-coated beads are used for normalization. FIG. 26 provides an overview of the present bead-based normalization protocol with NaOH-treated cellulose-coated beads versus a manual normalization protocol, and FIG. 27 provides more details on the steps of the bead-based protocol. FIGS. 18-23 show steps to evaluate the present bead-based normalization protocol with NaOH-treated cellulose beads, and FIGS. 24, 25, and 28 show the results with successful bead-based normalization using the presently described NaOH-treated cellulose-coated beads. Tables 4-7 below also provide further information on steps and equipment needed for both manual normalization and for bead-based normalization as described herein.

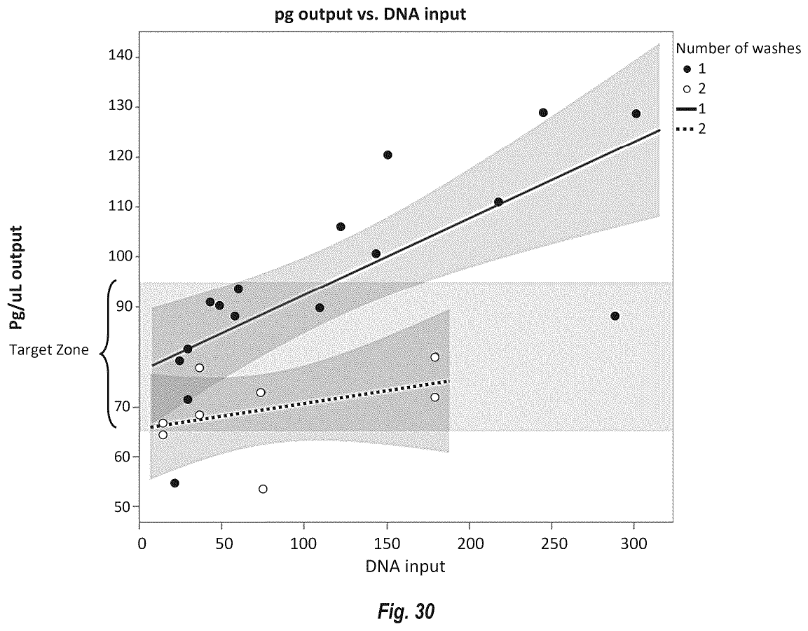

In some embodiments, untreated cellulose-coated beads are used for normalization. FIGS. 30-38 provide data on normalization with untreated cellulose-coated beads.

For applications with a small library input for normalization, untreated beads may be desired because a relatively larger number of beads for normalization may be useful so that the user can easily visualize the bead pellet. A higher number of beads will impact the reaction kinetics and beads, and DNA will collide more frequently when more beads are present. In other words, since the number of beads used for normalization will likely be higher when untreated beads are used, this can allow for quicker saturation of library binding to the beads. This faster saturation may be desired for a small-input library. Further, faster saturation means that the assay time may be shorter with less time needed for DNA binding to cellulose-coated beads.

In some embodiments, a method further comprises sequencing the normalized library. In some embodiments, the normalized library is amplified before the sequencing. In some embodiments, the normalized library is amplified and sequenced without determining the nucleic acid concentration of the normalized library. In some embodiments, the nucleic acid library is amplified before the normalizing is performed.

In some embodiments, normalizing the library improves the sequencing results. For example, the number of usable sequencing reads is higher for the normalized library as compared to the same library before the method of normalizing.

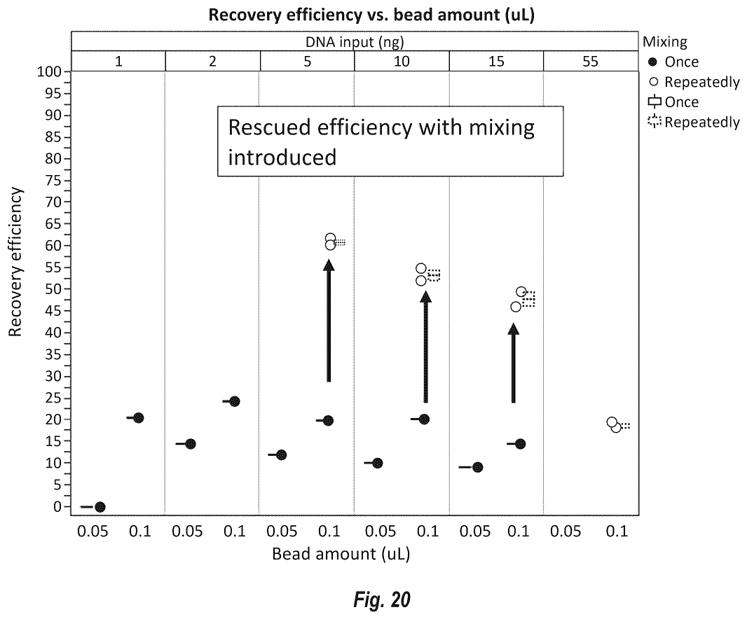

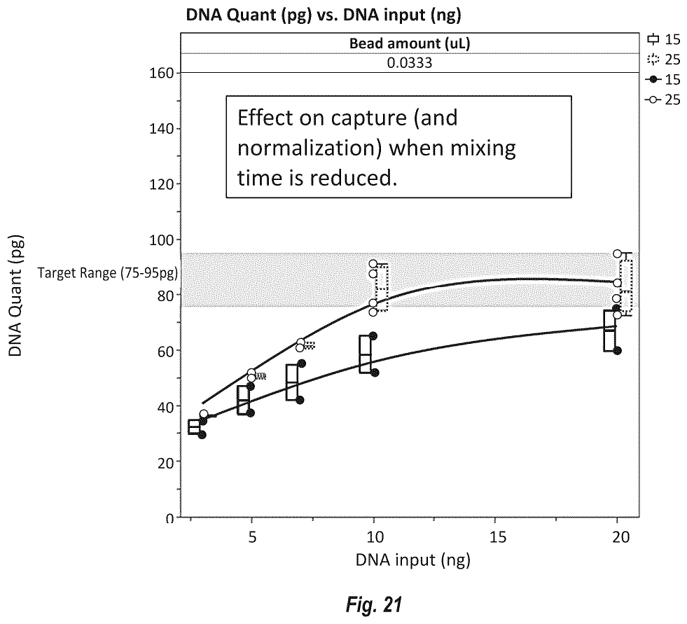

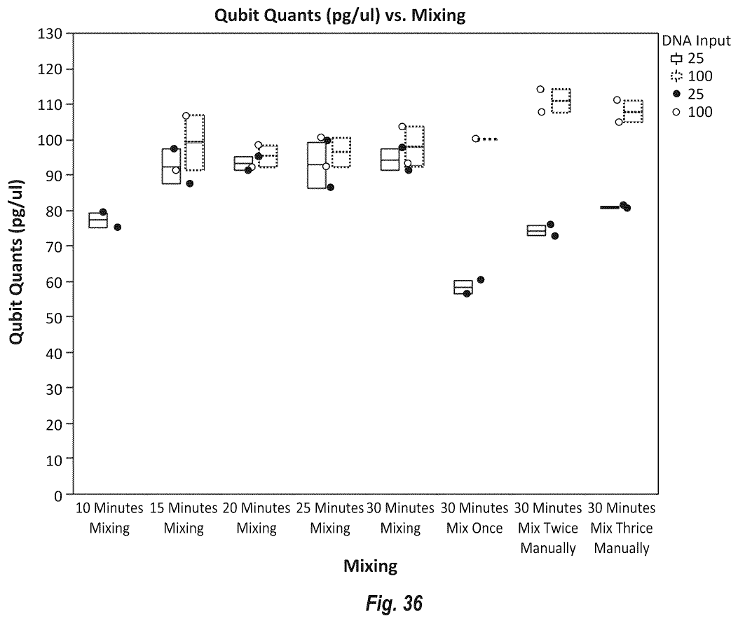

In some embodiments, the binding of the nucleic acid to cellulose-coated beads is performed with mixing. FIG. 20 shows improved efficiency of recovery with repeated mixing using treated cellulose-coated beads. In some embodiments, the mixing is performed using a bioshaker. Representative bioshakers include vortexers or mixers, such as an Eppendorf thermomixer. In some embodiments, the mixing is performed for 30 minutes or more. As shown in FIG. 21, longer mixing times may improve DNA capture during normalization. In some embodiments, for samples with small DNA input (such as 25 ng), incubation times in excess of 15 minutes may not be necessary for bead saturation. For example, FIG. 36 shows data on different mixing times with untreated cellulose-coated beads indicating that greater than 15 minutes of mixing with a bioshaker did not increase capture.

In some embodiments, the kinetics of DNA binding to cellulose-coated beads and the normalization efficiency of the reaction is based on the combination of variables of bead concentration, DNA amount, and reaction size. For example, more starting DNA likely speed up the reaction and ensure DNA binding saturation.

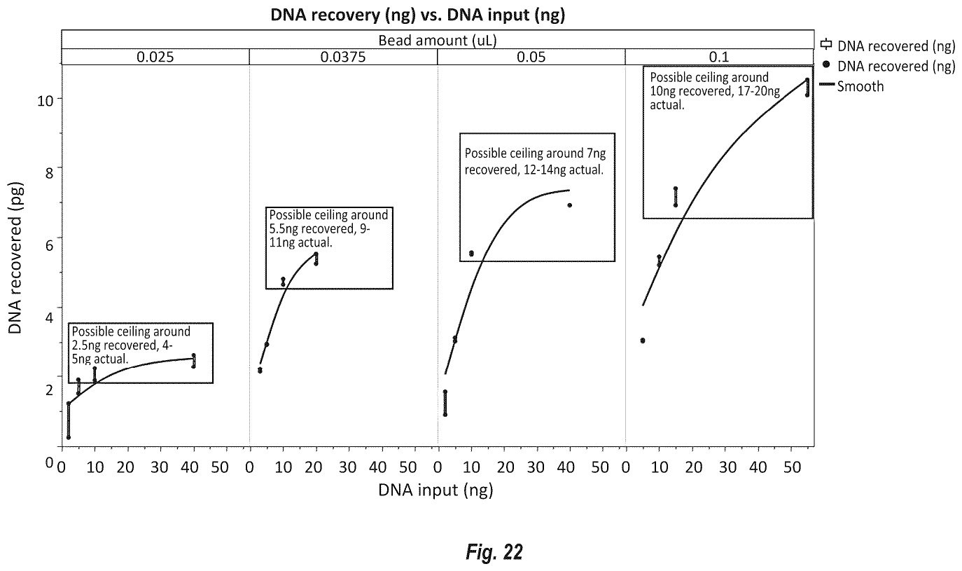

The present normalization method avoids sample loss that may be associated with dilution steps and may be especially helpful for samples with low amounts of nucleic acid. In some embodiments, the present methods have a stable recovery efficiency when normalizing over a range of DNA inputs (as shown in FIG. 18). In some embodiments, the nucleic acid library comprises 10 ng/μL or less, 1 ng/μL or less, 500 pg/μL or less, 200 pg/μL or less, or 100 pg/μL or less of nucleic acid. As shown in FIG. 22, if the sample comprises larger amounts of DNA, the amount of DNA recovered may be increased by using a larger number of beads.

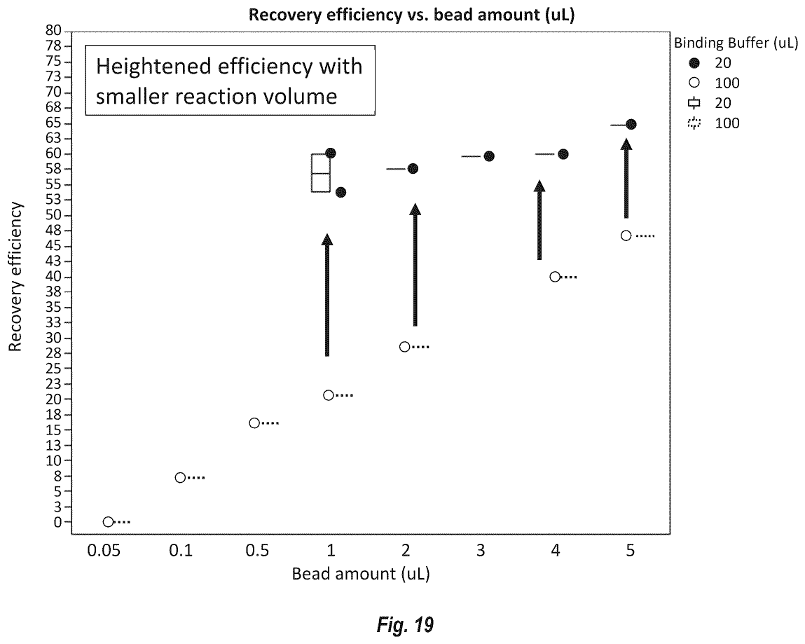

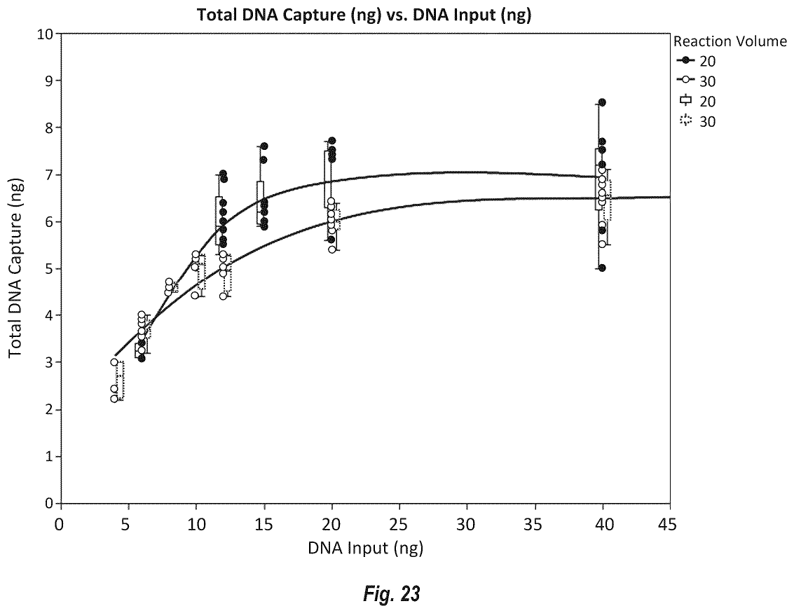

In some embodiments, the nucleic acid library for normalizing has a small volume. The ability to normalize a library with a small volume is presented in FIG. 19. In some embodiments, the library has a volume of 100 μL or less, 50 μL or less, or 20 μL or less. With the present methods, reactions volumes of 20 and 30 μL can show similar results (FIG. 23).

a. Fragment Size Distribution after Library Normalization

The size of library fragments comprised in a library after normalization may be influenced by the type of cellulose-coated bead described herein that is used. As used herein, an input library is a library before normalization, and the normalized library is a library that has subjected to normalization.

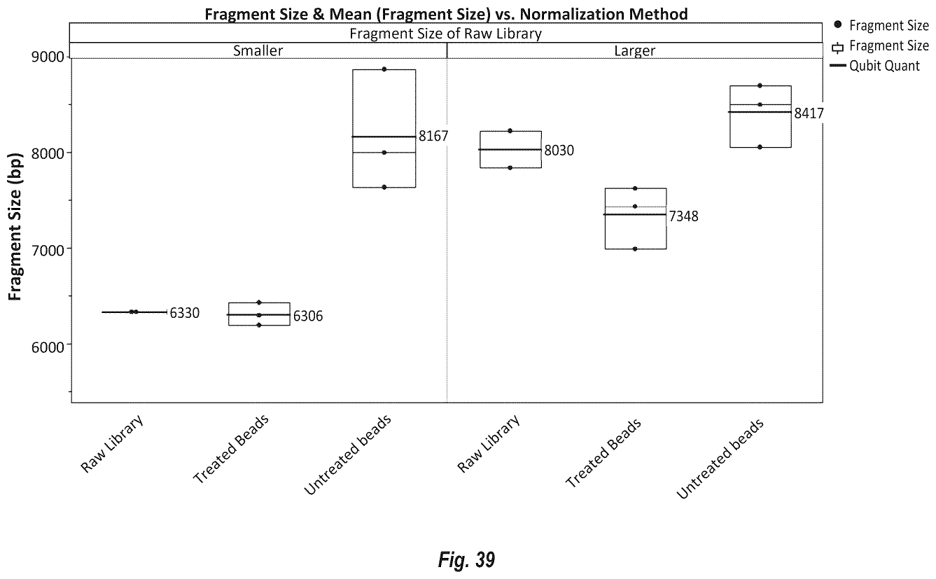

Using methods of normalization described herein, a user could either choose to prepare a normalized library comprising library fragments of a similar size as those fragments comprised the input library or choose to prepare a normalized library comprising library fragments that are larger than those fragments comprised in the input library. FIGS. 39-45 provide data on how untreated beads may lead to larger fragment size of normalized libraries, while NaOH-treated beads may lead to fragment size that is relatively unchanged from the library without normalization (i.e., a raw library).

For certain methods, a user may want the normalization to not affect fragment size. In some embodiments, the user wants the normalization process to not introduce any size bias.

In some methods, a normalized library comprising larger fragments may be preferred. For example, if a user is performing de novo sequencing with a metagenomic sample, it may be helpful to sequence a library comprising larger fragments to help identify an unknown pathogen from fragment sequences. In some embodiments, a user may want the normalization to produce a normalized library comprising a larger fragment size.

Methods of determining fragment size in a library would be well-known to those in the art, such as by using a Bioanalyzer. In some embodiments, the average fragment size comprised in the normalized library is the same or similar to the average fragment size comprised in the library before the normalizing, when the normalizing is performed with cellulose-coated beads prepared by a treatment with NaOH. In some embodiments, the average fragment size comprised in the normalized library is larger than the average fragment size comprised in the library before the normalizing, when the normalizing is performed with untreated cellulose-coated beads.

In some embodiments, the size difference between the same input library normalized with NaOH-treated beads versus normalized with untreated cellulose-coated beads is greater when the input library comprises fragments with a smaller average fragment size. In some embodiments, the size difference between the same input library normalized with NaOH-treated beads versus normalized with untreated cellulose-coated beads is less when the input library comprises fragments with a larger average fragment size. In other words, the choice of the bead used for normalization may be more important for libraries comprising small fragments. In some embodiments, a user may choose to normalize a library with untreated cellulose-coated beads to reduce the presence of small fragments in the normalized library.

In some embodiments, a library normalized with NaOH-treated cellulose-coated beads retains the maximum peak size and/or size distribution of the input library. In some embodiments, NaOH-treated beads normalize an input library without altering the maximum peak size and peak distribution of the library. In other words, the fragment size of a library normalized with NaOH-treated beads is generally similar to that of the input library. In some embodiments, the average fragment size of a library normalized using NaOH-treated beads differs by 15% or less, 10% or less, 5% or less, 4% or less, 3% or less, 2% or less, or 1% or less from the average fragment size of the input library before normalization.

In some embodiments, a library normalized with untreated cellulose-coated beads has a shifted maximum peak size and/or size distribution as compared to the input library. In some embodiments, a library normalized with untreated cellulose-coated beads has a larger maximum peak size as compared to the input library. In some embodiments, a library normalized with untreated cellulose-coated beads has a larger size distribution as compared to the input library. In some embodiments, a library normalized with untreated cellulose-coated beads comprises fragments with an average size that is greater than the average size of fragments comprised in the input library. In some embodiments, the average fragment size of a library normalized with untreated beads is 5% or more, 10% or more, 15% or more, 20% or more, 25% or more, or 30% or more greater than the average fragment size of the input library before normalization. In some embodiments, the maximum fragment size of a library normalized with untreated beads is 5% or more, 10% or more, 15% or more, 20% or more, 25% or more, or 30% or more greater than the maximum fragment size of the input library before normalization.

### B. GC Bias of Normalization

The distribution of AT-rich and GC-rich regions of nucleic acids may be referred to as GC content, and this distribution is uneven across the genomes of many organisms, including humans. Regions of high GC content are often poorly represented during sequencing, and this disproportionate coverage may be referred to as “GC bias” (see, for example, Gunasekera et al., PloS ONE 16 (6): e0253440 (2021)). GC bias may be introduced during library preparation (such as during PCR workflows), during hybrid capture, or during a sequencing run itself. GC bias in a sequencing run can reduce the amount of sequence data generated for analysis and can thus result in loss of important loci within the assembled genomes.

Approximately 95% of genome sequences have GC bias of 62% or less, and such sequences have generally been well-characterized in genome sequencing for diagnostics. In contrast, approximately 5% of genome sequences have GC bias of 62% or more, which may be referred to as “high GC regions” or “GC-heavy regions.” Such high GC bias regions have been poorly characterized by current genome sequencing methods.

In some embodiments, normalization with cellulose-coated beads (either untreated or treated with NaOH) may be used to prepare a GC-biased library. In some embodiments, cellulose-coated beads may be used to enrich for library fragments that have a GC content of 62% or more or a GC content of 60%-70%. In some embodiments, a normalized library has a greater percentage of library fragments that have a GC content of 62% or more or a GC content of 60%-70% as compared to the raw library (i.e., the same library before normalization).

In some embodiments, the sequencing results from a normalized library comprises more sequences from library fragments that have a GC content of 62% or more or a GC content of 60%-70% in comparison to the raw library. In some embodiments, sequencing results for certain fragments with a GC content of 62% or more or a GC content of 60%-70% are only obtained with a normalized library, and sequencing results are not obtained for these fragments with a raw library.

In some embodiments, library normalization as described herein may be used with a library preparation protocol without amplification prior to normalization.

In some embodiments, the relative sequencing coverage of fragments with a GC bias of 62% or more or with a GC bias of 60%-70% is higher for the normalized library as compared to the same nucleic acid library before the method of normalizing.

In some embodiments, the normalized library has 10% or more, 20% or more, 30% or more, 40% or more, 50% or more, 60% or more, 70% or more, or 80% or more greater sequencing coverage of fragments with a GC bias of 62% or more or with a GC bias of 60%-70% as compared to the same nucleic acid library before the method of normalizing.

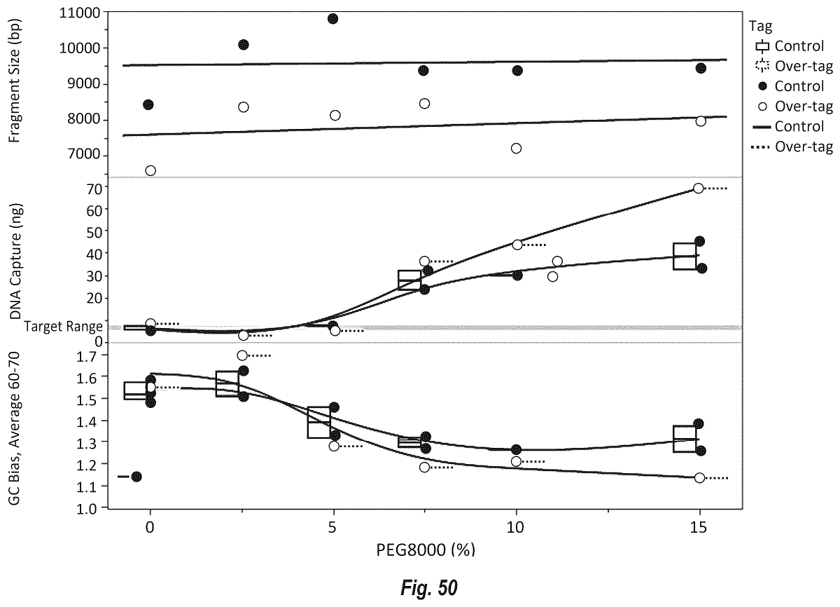

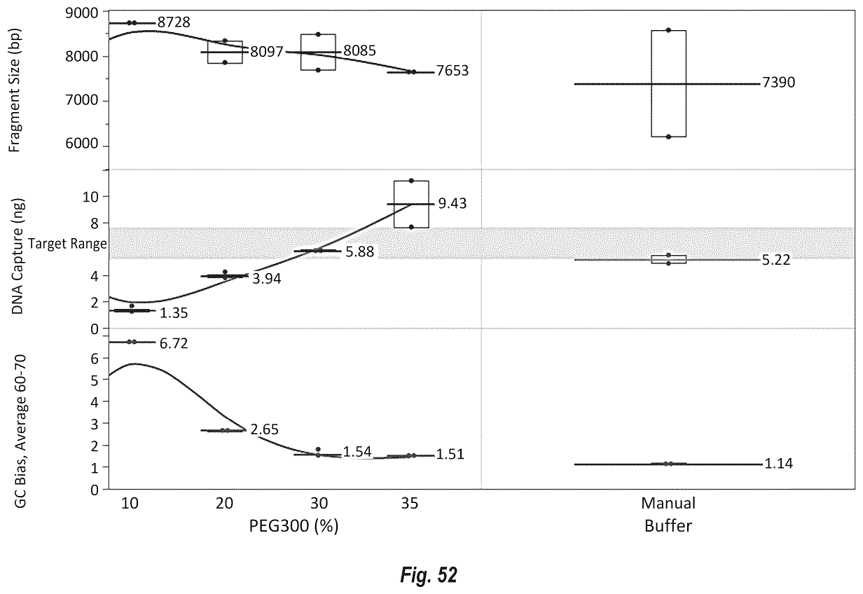

In some embodiments, a binding buffer with lower PEG concentration produces a normalized library with more GC bias, as shown in FIGS. 50 and 52. In some embodiments, the PEG concentration is 30% or less (weight/volume). In some embodiments, the PEG concentration is 10%-30% (weight/volume). In some embodiments, a higher concentration of GuSCN in a binding buffer promotes GC bias in a normalized library.

Certain binding buffers may promote GC bias when normalizing a nucleic acid library. In some embodiments, a binding buffer comprises 30% or less (weight/volume) of PEG200, PEG300, and/or PEG400; and 2-5M GuSCN. In some embodiments, the binding buffer comprises 10%-30% (weight/volume) of PEG200, PEG300, and/or PEG400; and 2-5M GuSCN. In some embodiments, the binding buffer comprises 3M or more GuSCN.

In some embodiments, a binding buffer comprising 30% or less or 10%-30% PEG is used for normalizing a nucleic acid library and the normalized library has a greater number of fragments with a GC bias of 62% or more or with a GC bias of 60%-70% in comparison to a normalized library wherein the binding buffer comprises more than 30% (weight/volume) of PEG200, PEG300, and/or PEG400.

In some embodiments, a binding buffer comprising 30% or less or 10%-30% PEG is used for normalizing a nucleic acid library and the normalized library has 10% or more, 20% or more, 30% or more, 40% or more, 50% or more, 60% or more, 70% or more, or 80% or more fragments having a GC bias of 62% or more or with a GC bias of 60%-70% in comparison to a normalized library wherein the binding buffer comprises more than 30% (weight/volume) of PEG200, PEG300, and/or PEG400.

### C. Tunable Normalization

As described above for extraction methods, the fragment size bound (and released) by treated cellulose-coated beads can be modified by varying the NaOH treatment (such as the time of treatment or the concentration of NaOH). In this way, a user can tune a method of normalizing a library by using treated beads that bind the desired ranges of fragment sizes.

In some embodiments, the treatment with NaOH used for the beads is selected to produce a desired level of yield of eluted nucleic acid and/or a desired fragment size of the normalized library.

In some embodiments, the desired characteristics of normalization are seen with untreated cellulose-coated beads. In other words, a user may choose to use treated or untreated cellulose-coated beads for normalization based on factors such as the concentration of libraries to be normalized and the desired normalization yield. For example, as shown in FIG. 29, normalization with untreated cellulose-coated beads led to significantly lower amounts of DNA recovery (i.e., lower yield of normalized libraries) as compared to normalization with NaOH-treated cellulose-coated beads. For applications where there is a small amount of DNA in starting libraries, a user may prefer to use untreated beads as a greater number of beads could be used for the normalization to achieve the desired yield of normalized library. In this way, the number of beads used for normalization may be large enough that the user can visualize the bead pellet. In other words, NaOH treatment may cause bead swelling and an increase in surface area that promotes higher DNA capture by each bead, the user may not require or desire this increased capture. For example, a user may want to normalize a library or other sample with only a small amount of DNA and thus prefers to use untreated beads.

In some embodiments, the desired level of yield of eluted nucleic acid is 40% or more, 50% or more, 60% or more, 70% or more, or 80% or more of the nucleic acid library. In some embodiments, treatment of the beads allows for an increase in the yield of the normalized library. In some embodiments, an increased yield of the normalized library is not needed and untreated beads may be preferred.

In some embodiments, normalizing increases the proportion of library fragments of a desired size. For example, if a user is normalizing a library prepared from cfDNA, the user may want to increase the proportion of small library fragments with the normalizing.

Alternatively, the user may normalize a library and want to retain large library fragments. For example, an exemplary library may be prepared from a metagenomics sample that comprises unpredicted or unknown species. In such a situation, normalization that retains large fragments may be desired to aid in analysis.

In some embodiments, the desired fragment size is 50-10000 base pairs. In some embodiments, the desired fragment size is 150-1000 base pairs.

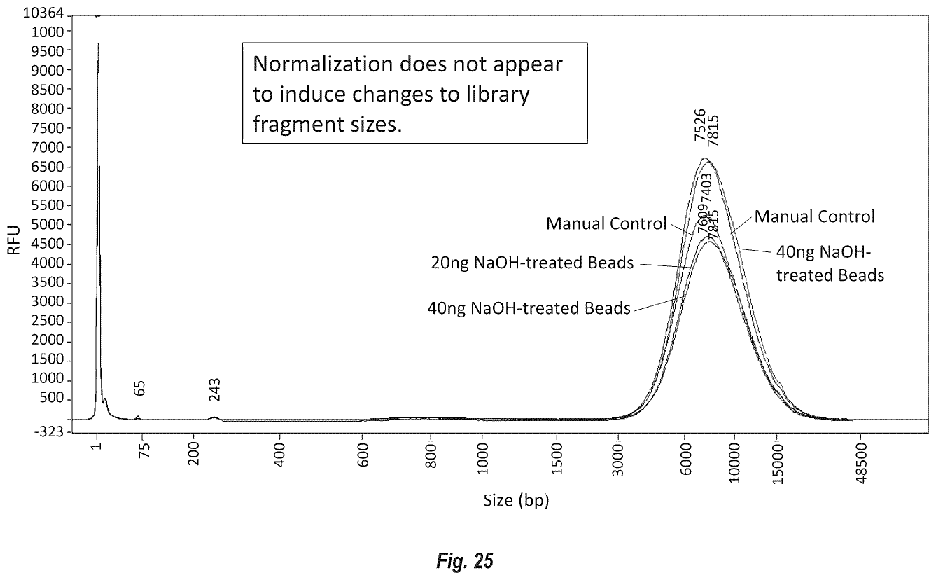

In some embodiments, present bead-based methods of normalization can yield normalized libraries of similar size to those prepared with standard manual normalization, as shown in FIG. 25.

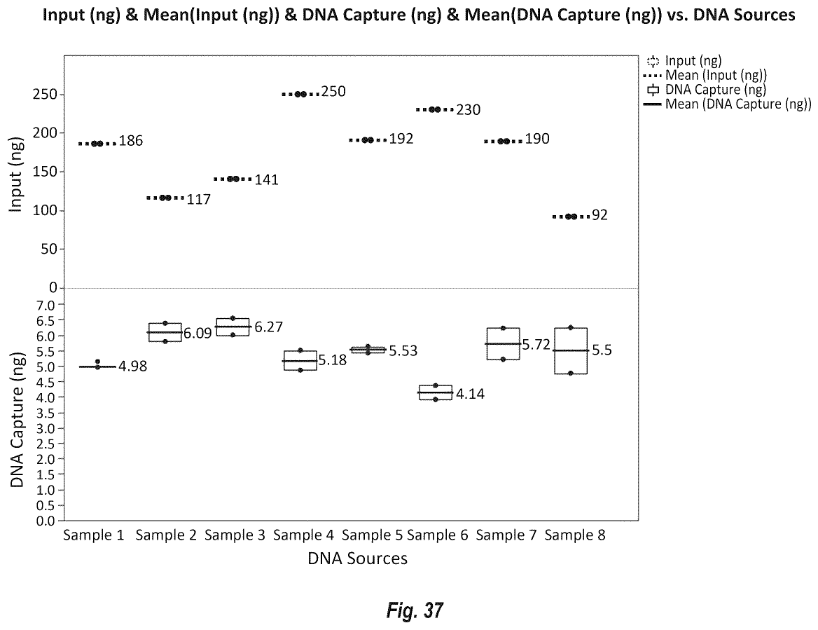

In some embodiments, normalization results with cellulose-coated beads were similar for libraries with DNA target size of 5 kb-10 kb or 25 kb-50 kb. FIG. 37 shows results on DNA capture during normalization with untreated cellulose-coated beads for sample sources with size ranging 25 kb-60 kb.

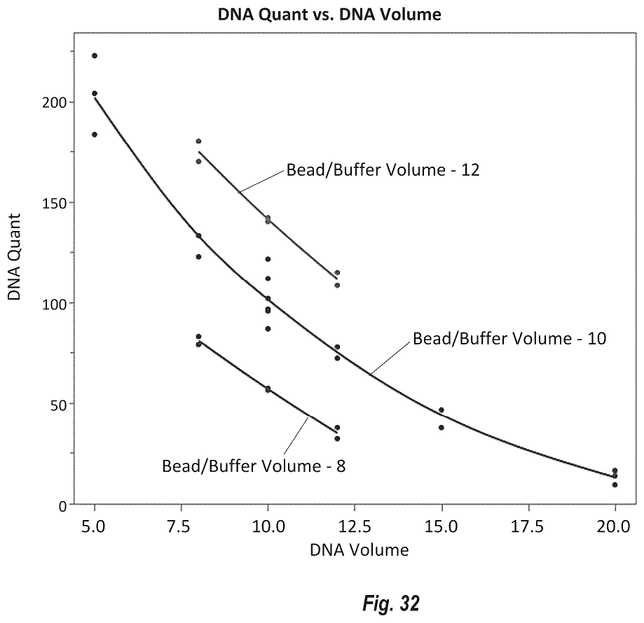

In some embodiments, pipetting volumes are controlled to avoid variation in final normalization yield. In some embodiments, even a 20% variation in any component can result in a significant change in the amount of DNA captured. FIG. 32 provides data on the fact that there may be a successful ratio of volumes for mixing the DNA with bead/buffer. In some embodiments, the ratio of bead/buffer components in the final mixture for binding of DNA has a sensitive apex spot (i.e., the relationship between concentration of DNA and bead/buffer than can achieve a desired binding reaction condition). In some embodiments, uncontrolled volume changes (such as +/−20% changes in volume of DNA or volume of bead/buffer) may upset this apex and result in an undesired change in DNA binding and normalized library yield.

The amount of PEG in a binding buffer can also be used to tune the binding to have the desired amount of DNA capture and library normalization efficiency. As shown in FIG. 33, a different concentration of the same PEG in a binding buffer can impact the capture efficiency (for example, a binding buffer with 30% PEG300 substantially increased the amount of DNA capture as compared to a binding buffer with 10% PEG300).

In some embodiments, methods of tuning the normalization method to a desired level of DNA capture (such as by altering the PEG concentration of the binding buffer) can allow the user to standardize the normalization yield between different batches of cellulose-coated beads. For example in FIG. 33, the capture of DNA is lower for Lot B beads in comparison to Lot A, but the level of capture with Lot B beads could be increased to the levels produced by Lot A by using a relatively higher concentration of PEG 300.

In other words, a normalization method may be tuned to a level that produces the desired results in downstream reactions (such as amplification and sequencing), and this level can be maintained in future experiments with different batches of cellulose-coated beads (that may have different intrinsic properties) by modifying treatment of the beads and/or altering the binding buffer characteristics.

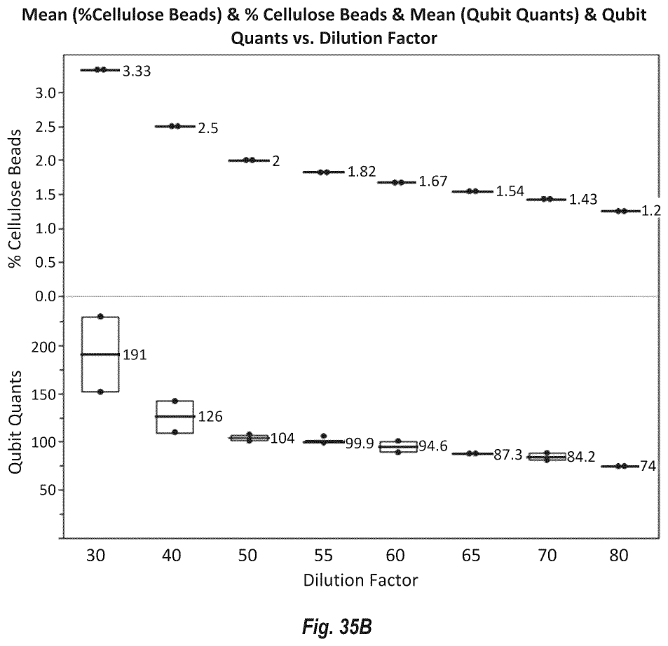

Further, the user can tune the amount of DNA capture during normalization based on a relatively linear relationship between the number of beads (i.e., the bead dilution). For example, FIGS. 35A and 35B show the relatively linear association between the amount of DNA captured and the percentage of cellulose-coated beads in the binding reaction.

### D. Wash Buffers for Normalization

In some embodiments, washing in normalization protocols helps to achieve a desired normalization target output. In some embodiments, the wash buffers for normalization are those described above for cfDNA extraction.

In some embodiments, washing after DNA binding to cellulose-coated beads (whether treated or untreated beads) helps reduce unwanted DNA carryover. In other words, washing helps to remove DNA that is not bound to beads before the elution of bound DNA. In some embodiments, increased washing reduces unwanted DNA carryover (i.e., carryover of unbound DNA).

In some embodiments, sufficient washing of the library products bound to cellulose-coated beads is achieved by using a relatively large volume of wash buffer. For example, a single wash with wash buffer may be performed for library products bound to cellulose-coated beads using 200 μL of wash buffer in a 96-well plate (i.e., a maximum volume wash). In some embodiments, washing with a large volume of wash buffer serves to reduce unwanted DNA carryover compared to washing with a smaller volume of wash buffer. In some embodiments, multiple wash steps serve to reduce unwanted DNA compared to a single wash step.

In some embodiments, increased washing (such as with multiple washes or a relatively larger volume of wash buffer) improves results when normalization is performed with untreated beads. In some embodiments, results for normalization with untreated cellulose-coated beads are improved by increased washing.

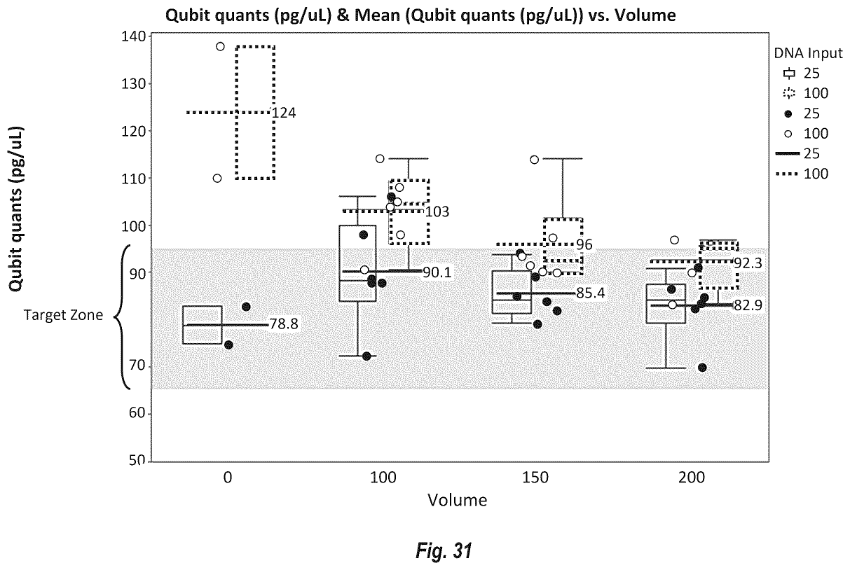

FIG. 30 shows representative data with 2 washes versus 1 wash for normalization with untreated cellulose-coated beads. FIG. 31 shows that potential carryover may be more pronounced for normalization when the library input is larger (i.e., when 100 ng of library DNA is used as input as compared to 25 ng of library DNA).

In some embodiments, a user may use multiple washes with a relatively large volume of wash buffer to reduce carryover as much as possible. In some embodiments, a user may choose multiple washes or a single high-volume wash for normalizing a large-input library.

### E. Normalization of Libraries Comprising Concatenated Sequencing Templates

In some embodiments, treated cellulose-coated beads are used to normalize libraries comprising concatenated sequencing templates. As used herein, “concatenated sequencing templates” refer to sequencing templates that comprise more than one insert sequence from a target nucleic acid (i.e., sequencing templates comprising multiple inserts).

A number of different methods have been developed as potential means of improving preparation of sequencing templates with multiple inserts, such as Duplex Sequencing (Schmitt, et al. Proc. Natl. Acad. Sci. U.S.A 109:14508-14513 (2012), Duplex Proximity Sequencing (Pro-Seq, as described in Pel et al. PLOS One 13:1-19 (2018)), CypherSeq (Gregory et al. Nucleic Acids Res. 44: e22 (2016)), o2n-seq (Wang et al. Nat. Commun. 8, 15335 (2017)), Circle Sequencing (Lou et al., Proc. Natl. Acad. Sci. U.S.A 110:19872-19877 (2013)), and Bot Sequencing (Hoang et al. Proc. Natl. Acad. Sci. U.S.A 113:9846-9851 (2016) and Abascal et al. Nature 593, 405-410 (2021)), each of which is incorporated herein in its entirety.

The Concatenating Original Duplex for Error Correction (CODEC) method recently described in Bae et al., bioRxiv, 10.1101/2021.06.11.448110, posted Jun. 12, 2021, which is incorporated herein by reference, involves preparing concatenated sequencing templates by physically linking both strands of double-stranded DNA for sequencing of a single duplex with a single read pair using specialized CODEC adapter complexes. The CODEC method can be used to identify non-canonical base-pairing that may be due to nucleobase damage or to a change comprised only in one strand of a double-stranded nucleic acid, as well as errors that may have been introduced during PCR amplification or sequencing.

Further, PCT Application No. PCT/US2021/055878, incorporated by reference herein in its entirety, describes a wide variety of different means to prepare concatenated sequencing templates via ligation of adapters or tagmentation.

While concatenated sequencing templates have a wide range of applications, such as to increase sequencing throughput (when templates comprise different inserts) or to allow for error correction (when templates comprise an insert sequence and a copy of the insert sequence), these templates have inherently high risk for being too large to be successfully amplified and/or sequenced. In some embodiments, use of normalization methods described herein improves sequencing results for concatenated sequencing templates by normalizing template size before amplifying and/or sequencing steps.

F. Normalization of Extracted cfDNA

In some embodiments, extracted cfDNA is normalized using cellulose-coated beads. As cfDNA can be highly variable in size, this normalizing can improve consistency of fragment size and yield of cfDNA samples.

In some embodiments, a method of normalizing extracted cfDNA comprises:
a. combining a solution comprising cfDNA with cellulose-coated beads, optionally wherein the cellulose-coated beads are prepared by a treatment with NaOH;
b. binding the cfDNA to the cellulose-coated beads in a binding buffer;
c. washing the bound cfDNA with a wash buffer; and
d. eluting the bound cfDNA with a resuspension buffer to prepare normalized cfDNA.

In some embodiments, the normalizing is performed with end-to-end automation. In some embodiments, the normalizing produces a desired level of yield of eluted cfDNA and/or a desired fragment size. In some embodiments, the desired fragment size is 75-300 base pairs. In some embodiments, the desired yield of eluted cfDNA is 5-50 ng.

In some embodiments, a nucleic acid library is prepared from the eluted cfDNA. In some embodiments, a nucleic acid like cfDNA is normalized and then a library is prepared. In some embodiments, the resulting library is then normalized.

G. Library Purification, Size Selection, and Normalization with Cellulose-Coated Beads

Sequencing data from cell-free DNA (cfDNA) have a number of important uses, such as evaluation circulating tumor DNA (ctDNA) and assessing fetal DNA using cfDNA prepared from a pregnant mother. However, such approaches often use shotgun library preparation, which can generate libraries requiring normalization and size selection for reliable sequencing data. As used herein, a “shotgun library” refers to a library prepared from a collection of random-sheared DNA molecules. Such shotgun libraries are generally characterized as having a broad range of insert sizes. This variability in insert sizes means that size selection for appropriate library fragment size can dramatically improve sequencing results.

For example, noninvasive diagnosis of fetal aneuploidies can be performed using shotgun libraries from cfDNA prepared from maternal blood. The detection includes the fetal trisomy and genome-wide fetal anomalies including chromosome number variant, partial duplications/deletions of all autosomes, and sex chromosome aneuploidy (Fan et al., PNAS 105 (42): 16266-16271 (2008), Pertile et al., Clin Chem 67 (9): 1210-1219 (2021).

However, shotgun libraries may need multiple steps of optimization to improve sequencing results, as shown in FIG. 60. For example, a purification step may be needed to separate the library from reagents used for preparing the library, such as library preparation enzymes and buffers, as these reagents may interfere with downstream sequencing reactions.

Further, size selection may be needed to improve sequencing results by enrichment of properly sized fragments for sequencing. This size selection can exclude very large fragments that will not properly sequence as well as short, unligated adapters that may be used in library preparation but that the user would not want to sequence.

In addition, normalization of libraries may be required to improve consistency in concentrations of different libraries in pooled samples. In the absence of normalization, libraries from some samples may overwhelm sequencing results while other samples may not be represented.

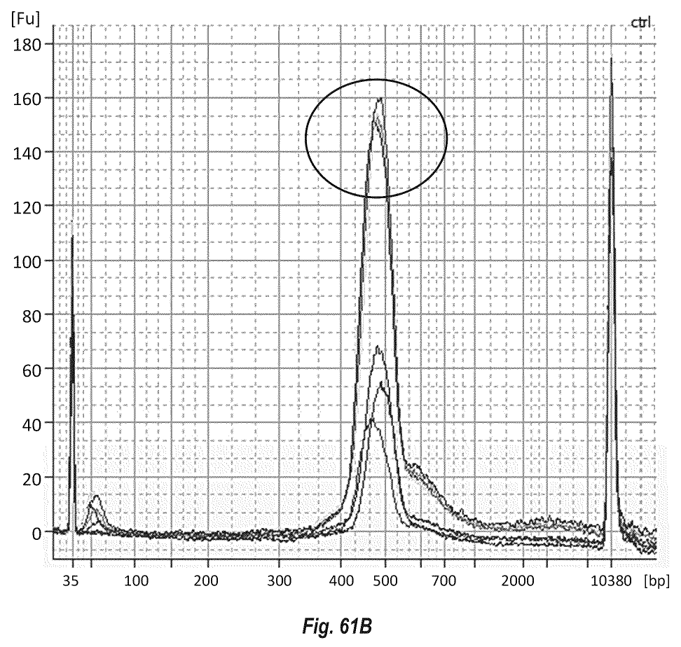

Cellulose-coated beads allow for a single bead to be used for purification, size selection, and normalization (process #2 in FIG. 60). Current protocols for shotgun libraries now use SPRI beads for purification/size selection followed by normalization with streptavidin pre-attached biotinylated beads (LNB1 beads, as shown in process #1 in FIG. 60), which requires extra time and cost for steps with two different bead types. Other beads, such as carboxylated beads, do not allow for purification, size selection, and normalization to be performed with a single type of bead. FIGS. 61A-65B confirm that sequencing data were similar for methods with cellulose-coated beads as compared to the 2-bead protocol with SPRI beads and LNB 1 beads. Accordingly, the presently described cellulose-coated beads other significant time and cost advantages for preparing libraries for sequencings, with particular advantages for shotgun libraries that generally have greater variability in fragment size and concentrations than other types of libraries.

In some embodiments, the normalizing separates reagents used for library preparation from the library. As such, normalizing can purify library fragments. In some embodiments, the normalizing produces a desired level of yield of eluted cfDNA and/or a desired fragment size. In some embodiments, the desired fragment size is 75-300 base pairs. In some embodiments, the desired yield of eluted cfDNA is 5-50 ng.

In some embodiments, a method of library purification, size selection, and normalization only uses cellulose-coated beads. In some embodiments, the method does not use carboxylate or streptavidin beads.

In some embodiments, the library is prepared from cfDNA. In some embodiments, the cfDNA comprises fetal cfDNA and/or circulating tumor DNA. In some embodiments, the library is a shotgun library.

In some embodiments, the nucleic acid library is prepared using one or more methods described herein. For example. cfDNA may be extracted from blood or plasma using cellulose-coated beads, a library prepared, and then the library is normalized using cellulose-coated beads.

IV. Kits for Extracting cfDNA and Normalizing Libraries

In some embodiments, kits comprising treated or untreated beads may be used for cfDNA extraction and/or library normalization. In some embodiments, different components of kits are included based on whether the kit is for extracting cfDNA or for normalizing libraries.

In some embodiments, a kit for extracting cfDNA from a sample or normalizing a library comprises (a) cellulose-coated beads prepared by treating with NaOH; (b) a binding buffer; (c) a wash buffer; and (d) a resuspension buffer. The treatment of the beads may be any described herein.

In some embodiments, a kit for normalizing a library comprises (a) untreated cellulose-coated beads; (b) a binding buffer; (c) a wash buffer; and (d) a resuspension buffer.

a. Kits for Extracting cfDNA

In some embodiments, the kit is for extracting cfDNA from a sample. In some embodiments a kit for extracting cfDNA from a sample further comprises proteinase K and/or ethanol. In some embodiments, the proteinase K may be used to remove histones from cfDNA. In some embodiments, the ethanol may be used for washing bound nucleic acid before eluting.

### B. Kits for Normalizing Libraries

In some embodiments, the kit is for normalizing a library. In some embodiments, a kit for normalizing a library may comprise treated cellulose-coated beads, such as beads treated with NaOH. In some embodiments, a kit for normalizing a library may comprise untreated cellulose-coated beads.

In some embodiments, a kit for normalizing a library further comprises ethanol and/or reagents for preparing a library for normalizing. In some embodiments, the ethanol may be used for washing bound nucleic acid before eluting.

The kit for normalizing a library may comprise any reagents for methods of library preparation. For example, the reagents for preparing a library may comprise tagmentation or ligation reagents. In some embodiments, the tagmentation reagents comprise bead-linked transposomes. The bead-linked transposomes may be those included in Nextera™ XT and Illumina® DNA Prep, (M) Tagmentation (formerly known as Nextera DNA Flex) kits.

### EXAMPLES

### Example 1. Extraction of cfDNA

Extraction of cfDNA from plasma was evaluated using a variety of different beads. Samples used for experiments consisted of pooled and individual plasma. Pooled plasma was generated using unaffected XX or XY plasma. Individual maternal plasma samples were NIPT residual aliquots. Results were evaluated using both manual extraction workflow and automation extraction coupled with sequencing.

All extractions were performed in 96-well lo-bind deep-well plate using 16 μL proteinase K, 14 μL magnetic beads, 900 μL plasma, and 800 μL of binding buffer. Upon incubation and removing supernatant on a plate magnet, the beads are subsequently washed with 1.5 mL of wash buffer 1 and 1.5 mL of wash buffer 2 (75% ethanol). The cfDNA is finally eluted off the beads with 60-70 μL of resuspension buffer (RSB). Extracted cfDNA was then quantified using the Qubit™ dsDNA HS Assay according to manufacturer protocol, using 20 μL sample for each measurement. The cfDNA was further analyzed using Agilent 2100 Bioanalyzer (BA) DNA High Sense following manufacturer's protocols to monitor fragment size and distribution.

Table 1 presents a summary of the present extraction workflow using treated cellulose-coated beads. Sample input types included pooled plasma, individual plasma, and artificial plasma. Upon completion of sequencing, the data were analyzed to evaluate performance equivalency with different reagents.

TABLE 1
Description of cfDNA extraction workflow

| Step | Description |
|---|---|
| Plate Proteinase K (for plasma) | Add 16 μL in Deep Well plate. |
| Plate magnetic beads | Add 14 μL in Deep Well plate. |
| Transfer DNA fragment | Add 900 μL DNA fragment to Deep Well plate. |
| Mix magnetic beads | Pipet mix DNA solution with magnetic beads |
| Transfer Binding Solution | Add 800 μL Binding Solution to Deep Well plate. |
| Digestion incubation | Mix reaction and incubate at RT for 10 minutes. |
| Magnet incubation | Transfer plate to magnet. Incubate for 10 min. |
| | Displace supernatant, incubate an additional 5 min. |
| Remove supernatant | Remove supernatant and dispense in liquid waste container. |
| Transfer Wash 1 | Add 1.5 mL Wash 1 to plate. |
| Magnet incubation | Transfer plate to magnet. Incubate for 5 min. |
| Remove Wash 1 | Remove Wash 1 and dispense in liquid waste container. |
| Transfer Wash 2 | Add 1.5 mL Wash 2 to plate. |
| Magnet incubation | Transfer plate to magnet. Incubate for 5 min. |
| Remove Wash 2 | Remove Wash 2 and dispense in liquid waste container. |
| Transfer Wash 2 | Add 1.5 mL Wash 2 to the plate. |
| Magnet incubation | Transfer plate to magnet. Incubate for 5 min. |
| Remove Wash 2 | Remove Wash 2 and dispense in liquid waste container 3x. |
| Transfer resuspension buffer | Add 70 μL RSB to the plate and mix. |
| (RSB) | |
| Elution incubation | Incubate off magnet for 5 min. |
| Magnet incubation | Transfer plate to magnet. Incubate for 2 min. |
| Transfer cfDNA | Transfer 60 μL supernatant to new PCR plate. |

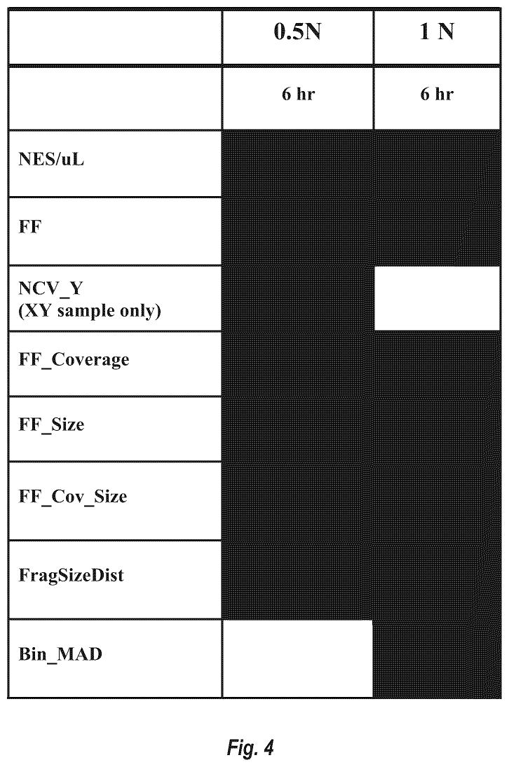

Values for performance equivalency (such as shown in FIGS. 3 and 4) were measured, including:
Usable sequencing reads per volume of library input (NES/μL TransferVol);
Normalized Chromosome_Y (Y sequencing count, NCV_Y);
Extracted DNA fragment size distribution=FragSizeDist; and
Fetal fraction (FF).

A variety of commercially available magnetic beads were evaluated, such as silica magnetic beads from GE Healthcare, AMO Lifescience, and Apostle Bio. The tested silica magnetic beads did not show acceptable cfDNA extraction performance (for example, no target DNA fragments were available or fragments were generally unwanted large DNA fragments based on Bioanalyzer results, data not shown).

Next, magnetic beads comprising cellulose were evaluated. Extraction from plasma yielded desired fragment sizes with minimal undesirable large fragments. However, the magnetic beads comprising cellulose had approximately 30% lower extraction yield as compared to comparator beads (Bioo Scientific beads comprised in NextPrep-Mag Kit 3825-01), as shown in FIG. 1A. In addition, the fragment size distribution for the magnetic beads comprising cellulose consistently extracted larger fragments as compared to the comparator beads (FIG. 2A). Inconsistencies in results from those with the comparator beads are not desired, especially for clinical or diagnostic assays since current methods of analysis might have to be redesigned for extraction with different yield or fragment size.

A wide range of different treatments were performed to attempt to adjust the physical and/or chemical properties of the cellulose beads and improve their profile. In other words, different treatments were assessed to determine a method of treating cellulose-coated beads that could be “tuned” to produce similar cfDNA extraction results to those seen for cfDNA extraction with the comparator beads.

0.01M HCl was first used to treat the magnetic beads comprising cellulose, but this treatment negatively impacted assay performance in term of cfDNA yield (data now shown). Treatments for 30 minutes with high concentration of acid (1M HCl), base (4M NaOH), or hot water all showed negative impact on the bead performance. Incubations of cellulose beads with 2M NaOH could also lead to dissolution of the cellulose matrix, which would eliminate cfDNA extraction onto the beads (data not shown). These results indicated that different treatments outside the scope of the claims recited herein decreased the yield of cfDNA extracted using magnetic beads comprising cellulose and were not appropriate.

After lack of success with other conditions, a lower concentration of 1M NaOH treatment for 60 hours followed by 3 rounds of wash with 1M Tris (pH 7.5) was evaluated. Beads treated with 1M NaOH for 60 hours specifically showed promising results. After this treatment and washing steps, the treated magnetic beads comprising cellulose showed slightly better yield than comparator beads (FIG. 1B). Further, these treated beads had significant extraction yield improvement compared to the untreated magnetic beads comprising cellulose (FIG. 1A). Consistent results with the 1M NaOH treatment were seen with three different users on different days (data not shown), indicating reproducible improvement in cfDNA extraction after treatment of beads comprising cellulose.

In addition, the fragment size distribution was similar for cellulose-coated magnetic beads treated with 1M NaOH for 60 hours as compared to the comparator beads (FIGS. 2A and 2B). These results indicated that the fragment distribution was similar for the treated cellulose-coated beads as compared to the comparator beads.

To test if the effects of this NaOH treatment is reversible by lowering the pH, acid treatment was performed after treatment of magnetic cellulose-coated beads with 1M NaOH. No drop in yield was seen after treatment with 0.01M HCl after initial treatment with 1M NaOH (data not shown), which confirmed that the NaOH treatment process is not reversible and that the cellulose resin comprised in the magnetic bead is stable after NaOH treatment.

Using 1M NaOH for 60 hours as a starting point, further evaluation was performed by varying both the NaOH concentration and treatment duration to evaluate yield in comparison to comparator beads. As shown in FIG. 8, a gradual decrease in yield was seen at concentrations above 0.8M NaOH. These data indicate minimal damage to the cellulose of treated beads at NaOH concentrations below 0.8M NaOH. The data suggested that a useful NaOH concentration could be 0.5M. Comparison of different times of 0.5M NaOH treatment indicated that similar results were seen with treatments between 12 hours and 24 hours (FIG. 9).

Treatment with 0.5M NaOH for 6 hours produced beads for efficient cfDNA yield with a manufacturing-friendly process. Results with artificial plasma (FIG. 3) showed that treatment with 0.5M NaOH or 1M NaOH for treatments of 16 hours showed acceptable results. Overall results in FIG. 3 with artificial plasma indicated that the 0.5M NaOH treatment is more robust than 1M NaOH treatment, as it has a larger time window for treatment and has more metrics that align with comparator beads. The fragment size distribution with artificial plasma did remain distinct from comparator beads for both treatment concentrations over time.

Further, results from plasma using beads with 6-hour treatments with 0.5M NaOH or 1M NaOH also showed acceptable results (FIG. 4). Overall results with real plasma indicated that both 0.5M and 1M concentration treatment of magnetic cellulose-coated beads for 6 hours or 8 hours is effective in providing the similar results to comparator beads. Further, the 0.5M NaOH-treated beads showed similar results in Y sequencing count (NCV_Y) metric to the comparator beads, which would be a measure necessarily assessed in fetal cfDNA diagnostic assays.

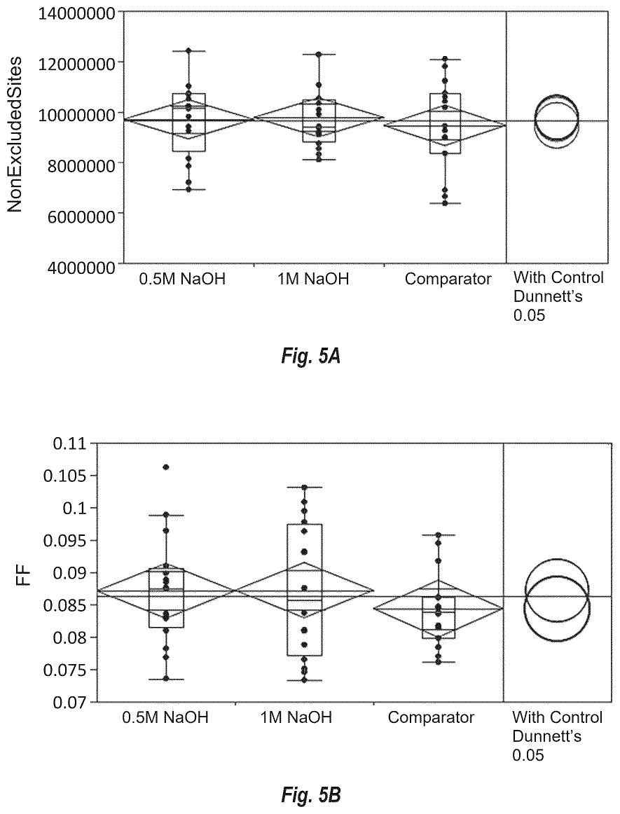

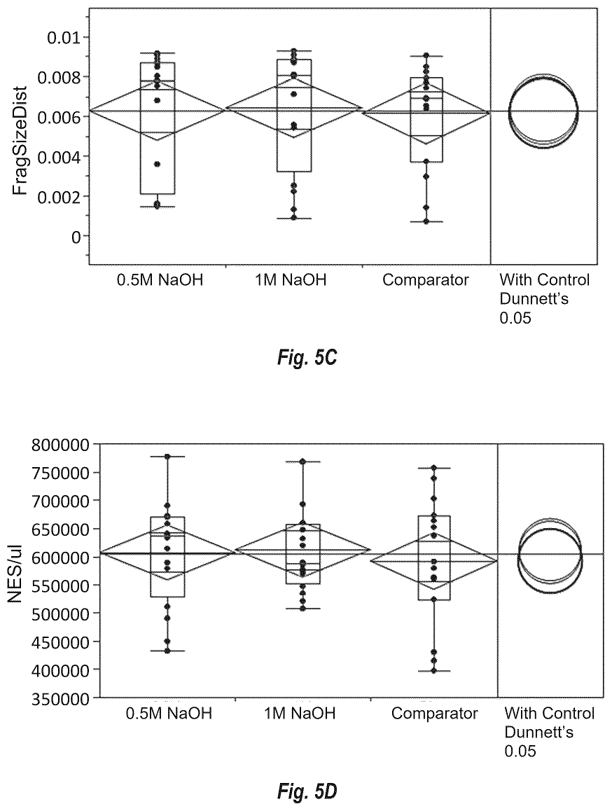

Four different lots of magnetic beads comprising cellulose were each treated with 0.5M NaOH for 6 hours, and results showed consistent sequencing results from real plasma. Further, across experiments and different users, cellulose beads treated with 0.5M NaOH for 6 hours or 8 hours showed equivalent performance to comparator beads for tested sequencing metrics. For example, non-excluded sites (NES, i.e., sequencing results that were associated with cfDNA), fetal fraction (FF), extracted DNA fragment size distribution (FragSizeDist), and usable sequencing reads per volume of library input (NES/μL TransferVol) were all comparable for comparator beads or cellulose-coated beads treated with 1M or 0.5M NaOH (FIGS. 5A-5D). Measures of NES are important comparators for different means of extracting cfDNA, as all means of extracting cfDNA can also extract other nucleic acids (such as genomic DNA) and measures of NES allow for direct comparison between different methods of extracting cfDNA.

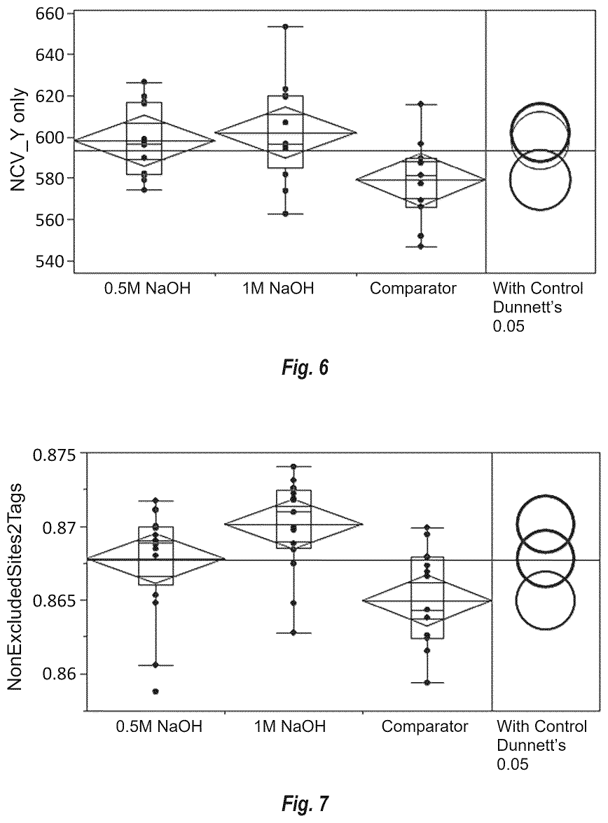

Measures of normalized chromosome Y (i.e., Y sequencing count, NCV_Y, FIG. 6) and non-excluded sites2Tags (a measure of reads useful for NIPT analysis per the total reads, FIG. 7) are other important measure for comparing different means of extracting cfDNA, especially for NIPT analysis. For both of these measures, results with the 0.5M NaOH treatment were slightly more consistent with the comparator beads than 1M NaOH treatment.

While both 0.5M NaOH-treated and 1M NaOH-treated beads can be used for cfDNA extraction, further experiments were done with 0.5M NaOH-treated beads as assays as analysis had been evaluated for results from the comparator beads. The results in FIGS. 5A-7 show that one skilled in the art can “tune” the properties of the NaOH-treated beads to their desired level of activity based on user preference and methodologies already in place for other means of cfDNA extraction. One skilled in the art could determine the desired level of extraction yield, fragment size, and other parameters, and then the user could determine what NaOH treatment is most convenient for them and gives them desired results. For example, standard shifts at manufacturing facilities may be 8 hours, and a NaOH treatment for this time-period may be convenient for commercial production.

Treated beads were stored in a simple storage buffer to avoid any impact on downstream workflows. Sodium azide was included for magnetic bead storage to prevent microbiome growth. Treated beads were stored in a buffer comprising 0.1×TE with 0.02% sodium azide.

### Example 2. Binding Buffer Evaluation

Evaluation was also performed on the binding buffer for cfDNA binding to the cellulose-coated beads. DNA can bind to certain types of particles in presence of high concentration of chaotropic agent (e.g., 5M-8M guanidinium thiocyanate (GuSCN)). Therefore, the first version of binding buffer (#1) was prepared with 7M GuSCN with 15% (w/v) PEG 8000.

The initial binding buffer (#1) yielded no fragment at target size, but large amounts of a much longer fragment. To fundamentally change the performance, low molecular weight PEG (PEG 300) was used in the buffer to replace PEG 8000. To achieve similar viscosity, the PEG 300 concentration was increased to 35%. To avoid a heating step during buffer preparation, the GuSCN concentration was also lowered to 4M. Beads treated with 0.5M NaOH for 6 hours together with a binding buffer with 35% PEG 300 and 4M GuSCN (binding buffer #2) yielded fragments with similar size to those extracted with comparator beads used with comparator buffer. Accordingly, binding buffer #2 helped to decrease unwanted contamination from large fragments of cfDNA.

Further versions of wash buffers and binding buffers were tested and showed acceptable results with Tris 20, pH 6.8, as well as Tween-20 (w/v) from 0-0.02% (data not shown). For example, 33% or greater PEG300 with 4M GuSCN and 3M or greater GuSCN with 35% PEG300 both showed acceptable results. In addition, wash buffers with lower PEG300 and GuSCN concentrations, such as 1M GuSCN and 15% PEG300 were also acceptable (data not shown). Testing of pH also showed that functional performance

differences were not seen over a range of pH 5-9, indicating that cfDNA binding is robust across a range of pH.

### Example 3. Evaluation of cfDNA Extraction from Plasma Versus Comparator

A variety of modifications and controls were performed to evaluate cfDNA extraction with treated cellulose-coated beads. Extractions were evaluated using protocols with either plasma or blood (FIG. 11) as a starting material. As outlined in FIG. 10, current methods of cfDNA extraction require plasma preparation, but methods described herein also evaluated cfDNA extraction directly from plasma, which could eliminate the requirement for plasma preparation.

For example, Bioanalyzer results were compared for cfDNA preparation from patient plasma between the present method of extraction from plasma using treated cellulose-coated beads (FIG. 12A) versus extraction using the Qiagen QIAmp Circulating Nucleic Kit (CNA) (FIG. 12B). The bead-based protocol followed the steps outlined in the protocol shown in FIG. 11 for cfDNA extraction from plasma.

Table 2 summarizes the similar results seen between the present bead-based extraction with treated cellulose-coated beads and an extraction with the Qiagen CNA kit (Qiagen Catalog No. 55114). Accordingly, the present extraction method produced similar cfDNA extraction as a standard comparator.

TABLE 2
Comparison of present bead-based extraction
versus Qiagen CNA comparator

| | Qiagen CNA % | Treated Cellulose-Coated |
|---|---|---|
| | of total | Bead % of total |
| cfDNA (100-200 bp on | 55.6% | 60.9% |
| Bioanalyzer or 75-250 on | | |
| Agilent Fragment Analyzer) | | |
| Di-nuc (300-500 bp) | 13.4% | 9.1% |
| Tri-nuc (500-700 bp) | 16.7% | 24.1% |
| HMW (700 bp+) | 8.7% | 5.9% |

### Example 4. Evaluation of cfDNA from Blood

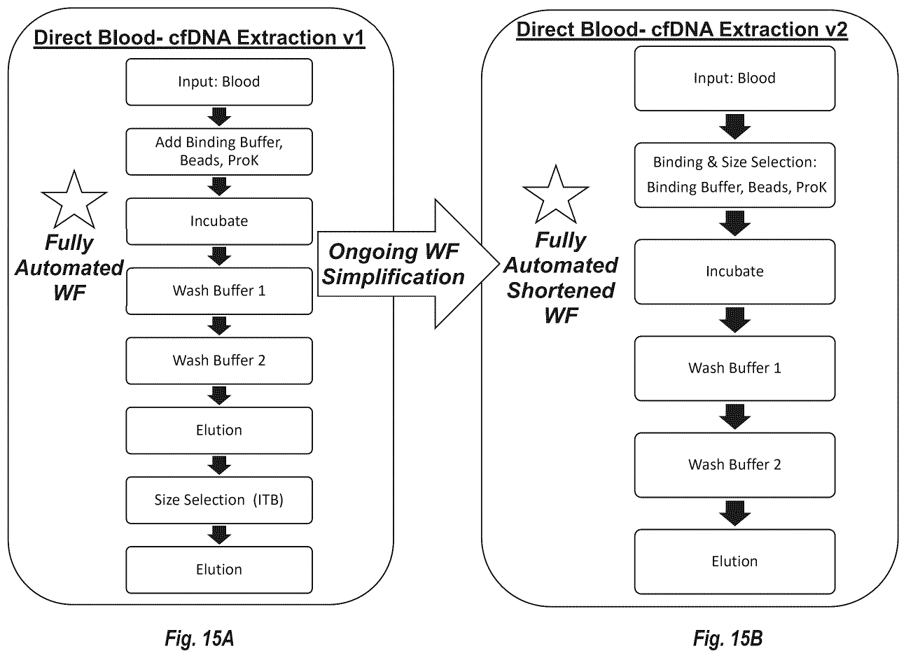

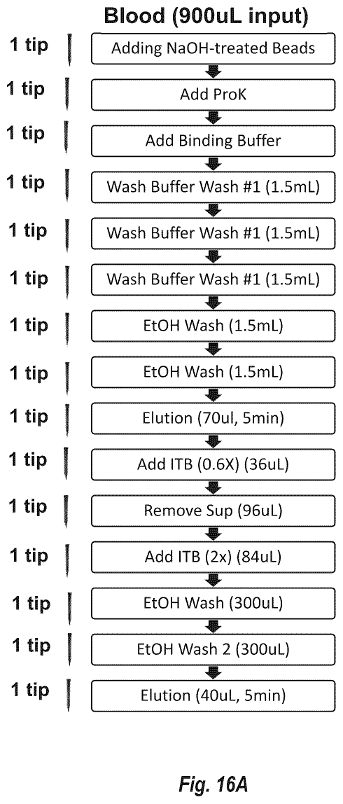

Direct extraction of cfDNA from blood has several advantages over present methods of extraction from plasma. For example, plasma preparation tubes (such as Streck tubes) are expensive and add to the costs of cfDNA analysis, but a method of extraction from blood could potentially use a tube with lyophilized beads and binding buffer. Further, removing manual steps of plasma preparation (as shown in FIG. 10) could eliminate the need for centrifugation and replace with automation-friendly magnetic bead extraction steps. A method of extraction from blood, however, would need to have high yield and deal with potential contamination from large genomic DNA (gDNA). FIG. 15 shows a fully automated extraction workflow from blood (including size-selection with Illumina Tune Beads (ITB)), as well as a shortened, fully automated workflow from blood wherein size selection can be performed during the binding step.

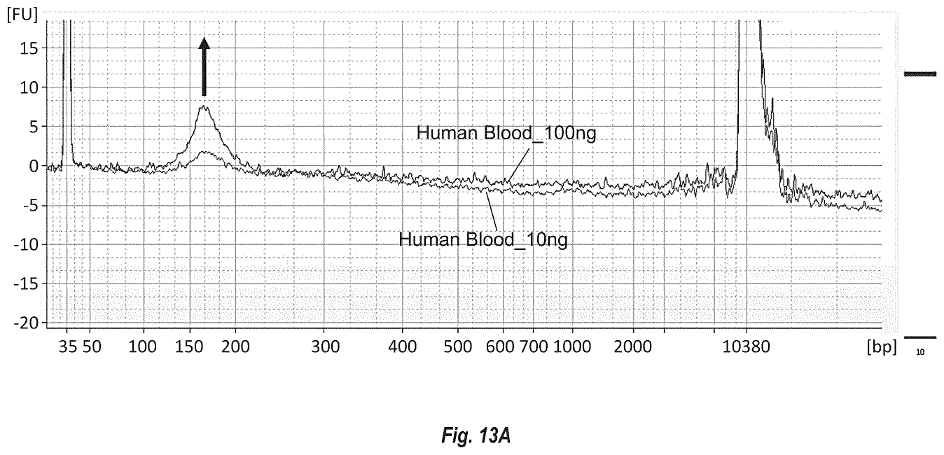

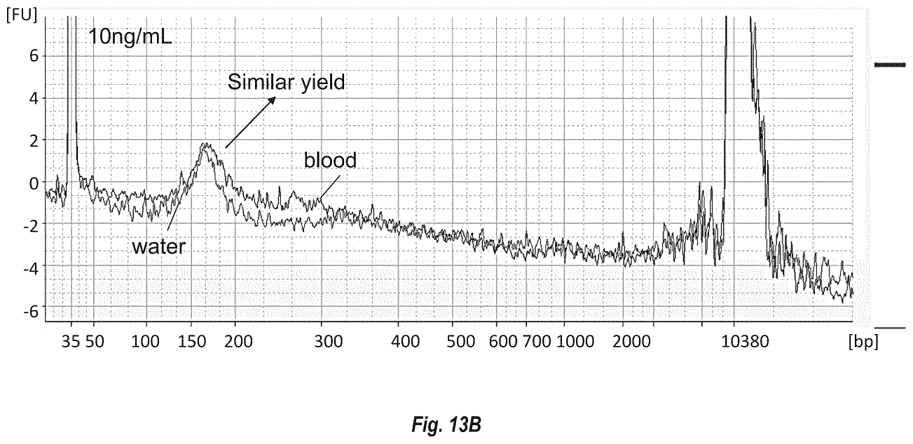

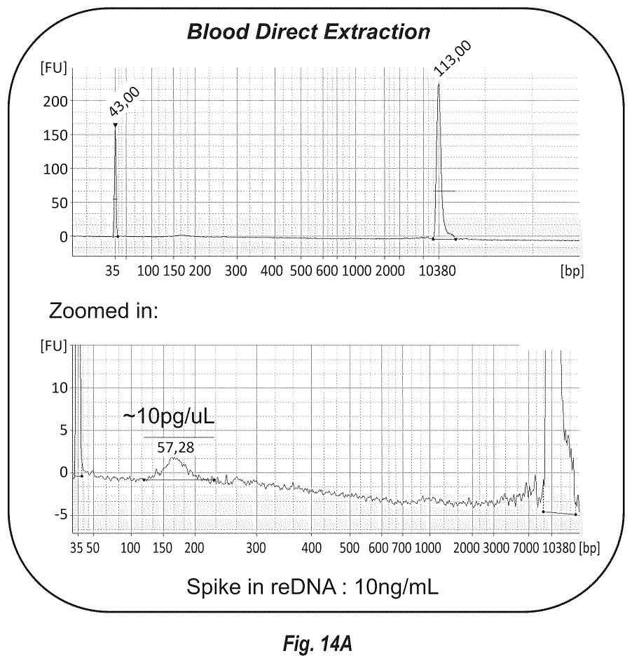

FIG. 13A shows extraction of spiked recombinant DNA (either 100 ng/ml or 10 ng/ml) from blood using treated cellulose-coated beads. The ability to extract the recombinant DNA from blood (as indicated by peaks for both recombinant DNA concentrations) indicates the possibility that issues of using blood as a starting point could be overcome. Further, FIG. 13B shows that similar yield of spiked recombinant DNA from either blood or water.

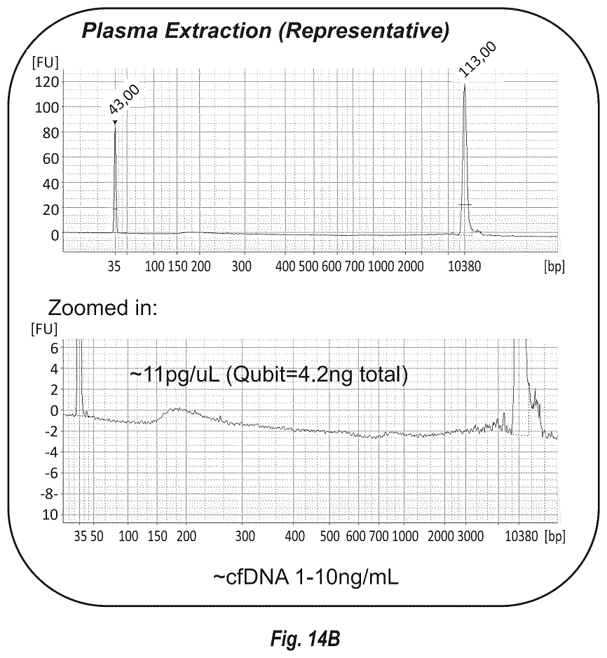

FIGS. 14A and 14B shows Bioanalyzer results indicating that the peak of spiked recombinant DNA was similar with the present extraction from blood was similar to the peak with cfDNA extraction from plasma with comparator beads. These results indicated that direct blood extraction with treated cellulose-coated beads may be a viable approach to avoid plasma preparation.

Results showing success of DNA extraction directly from blood suggest that such automated protocols may be able to replace cfDNA extraction methods using plasma. A comparison of potential workflows is shown in FIGS. 16A and 16B, with FIG. 16A showing a method with size selection using ITB. It should be noted that the plasma protocol shown in FIG. 16B begins after preparation of plasma, which is itself a time-consuming step.

FIG. 17 shows how the ITB-based size selection outlined in FIG. 16A may be performed. After elution of cfDNA from treated cellulose-coated beads, the eluate is mixed with ITB at 0.6× concentration. This step preferentially binds to high-molecular weight DNA (HMW DNA) or genomic DNA (gDNA), and the pellet comprising the bound beads is discarded. Next, ITB at 2× concentration is added to bind to remaining cfDNA (which has a fragment size of approximately 200 base pairs (bp)). The unbound supernatant is discarded to help remove proteins and other contaminants, and the beads bound to cfDNA are collected and washed. Then, the cfDNA is eluted off the beads and ready for analysis, such as sequencing. This size selection with ITB helps to remove HMW DNA and gDNA that might otherwise contaminate cfDNA extractions from blood and obscure sequencing results from cfDNA.

### Example 5. Evaluation of Parameters for Normalization with NaOH-Treated Cellulose-Coated Beads

Normalization of a library after its preparation is an important step to improve sequencing results by controlling for wide variation between library fragments for sequencing. Specifically, sequencing using high-density flowcells often requires normalization for high-quality data.

A wide variety of different parameters can impact whether certain treated cellulose-coated beads can be used to normalize small amounts of high-molecular weight DNA in a small amount of volume, and experiments were performed to determine the best conditions for normalization.

The DNA binding efficiency of NaOH-treated cellulose-coated beads treated as described above in Example 1 was assessed. Across several incubation periods and DNA inputs, the efficiency of bead capture was shown to be 40-60% of the input concentration regardless of the amount of DNA input (ng) or the resultant DNA output (pg), as shown in FIG. 18. Typically the recovery efficiency was maintained across incubation times and across DNA input concentrations. These data indicate that the present treated beads comprising cellulose allow for a robust method that normalizes upwards and throttles down depending upon the amount of starting DNA.

The effect of reaction volume was also assessed. Generally at lower amounts of beads in a reaction, the reaction volume becomes more important to overall function. When reducing beads and maintaining a larger (such as a 100 μL) reaction volume, the recovery efficiency can rapidly decrease (FIG. 19). When these same conditions have a reduced reaction volume, the efficiency is rescued. Therefore, reduction in the sensitivity of the assay to the number of beads used is important.

When there is a lower number of beads in a reaction, the reaction mixing may play a large role in DNA capture to maintain recovery efficiency during cfDNA extraction. Even with a reduced reaction volume, a large drop in beads per reaction may require more mixing to encourage DNA binding. Preliminary mixing was by pipette but mixing using a bioshaker at 1800 rpm results in better recovery efficiency (FIG. 20). The core of the presently tested cellulose-coated beads comprises 45%-55% iron oxide with a particle mass of 110-140 mg/mL, and the relative heaviness of these beads may improve mixing.

Mixing times also impacted the recovery efficiency. If mixing is reduced, the rate of DNA capture was likewise reduced. FIG. 21 shows the impact of DNA normalization when mixing is reduced from 25 to 15 minutes. Higher recovery efficiency is seen with mixing for 25 minutes or more.

The binding efficiency of NaOH-treated cellulose-coated beads was also assessed. As the number of beads per reaction is lowered, the DNA captured begins to level off at a proportionally lower amount, as shown in FIG. 22. At 0.1 μL of beads, a possible leveling-off of DNA captured was seen at approximately 17-20 ng. At 0.025 μL of beads, a possible leveling-off of DNA captured was seen at approximately 4-5 ng of DNA. These results indicate that libraries with large amounts of DNA may be better normalized with a larger amount of treated cellulose-coated beads.

According to a design of experiments (DOE) analysis, an even stronger impact of DNA capture is the final binding buffer content. Buffer content evaluation is important, and the binding buffer described in Example 2 can improve results over other buffers.

A mastermixing strategy was determined based on the results of FIGS. 18-23. All components should be mixed, preferably with a bioshaker. A binding buffer (as described in Example 2) should be used. The number of beads used should be increased (increase up to 0.04 μL per reaction).

Further, results with the mastermixing strategy showed that DNA saturates earlier and more evenly when reaction volume is kept low, 20 μL total rather than 30 μL total (FIG. 23). These results indicate that smaller reaction volumes may improve capture and DNA recovery from samples with small amounts of DNA when normalizing using NaOH-treated cellulose-coated beads.

### Example 6. Protocol for Bead-Based Normalization with NaOH-Treated Cellulose Beads

A protocol was developed for bead-based normalization of cfDNA using NaOH-treated magnetic cellulose-coated beads and compared to a standard manual method of normalization. An overview of manual normalization versus the present bead-based normalization is shown in FIG. 26. An important difference is that manual normalization requires measurement of DNA concentration, while the present bead-based normalization protocol does not. This is an important advantage to reduce hands-on time and increase the ability to automate a normalization protocol.

Prior to bead-based normalization, a preparation of 10× diluted beads and a mastermix was performed. To prepare 10× diluted beads, 50 μL of treated beads were well-mixed with 450 μL of 18% PEG300 solution was added to the bead solution. A master mix was then prepared as outlined in Table 3.

TABLE 3
Preparation of mastermix comprising beads

| | μL to combine | μL to combine for |
|---|---|---|
| Prepare Mastermix | for 1 sample | 16 samples |
| Binding Buffer | 8.8 | 140.8 |
| 10X Diluted Bead | 0.44 | 7.04 |
| RSB | 1.76 | 28.16 |
| Total (with | 11 | 176 |
| overage) | | |

RSB = resuspension buffer

FIG. 27 summarizes the steps in a representative normalization protocol. First, 10 μL of DNA, containing at least 10 ng of DNA, is added to each well of a plate. The prepared mastermix from Table 3 is vortexed until homogeneous immediately prior to use. 10 μL of prepared mastermix is pipetted into each well containing 10 μL of DNA. The plate is sealed with microseal and placed on a bioshaker for 30 minutes at 1800 rpm.

The plate is removed from the bioshaker and placed on a plate magnet for 1 minute. The supernatant is gently removed, taking care not to disturb beads. The plate is removed from the plate magnet and 150 μL of wash buffer 1 is immediately added to each well (i.e., Wash Step #1). Using a p200 pipette set to 140 μL, pipette mixing is performed for each well at least 10 times. The plate is then placed on a plate magnet for 1 minute.

After Wash Step #1, the supernatant is gently removed, taking care not to disturb beads. The plate is removed from the plate magnet. Immediately, 150 μL of 80% EtOH is added to each well (i.e., Wash Step #2). Using a p200 pipette set to 140 μL, each well is pipette mixed at least 10 times. The plate is placed on plate magnet for 1 minute. The supernatant is gently removed, taking care not to disturb beads. The plate is briefly spun down and a p20 pipettor is used to remove excess liquid. The plate is allowed to dry for 2 minutes.

Final elution into RSB is then performed with 80 μL of RSB added to each sample well. Using a pipette set to 50 μL, each sample is pipette mixed at least 10 times and allowed to incubate for 10 minutes. The plate is placed on a plate magnet for 1 minute, and 70 μL of supernatant is then transferred to a new well. The library can then be amplified and sequenced.

FIG. 26 provides an overview of the differences between this present bead-based normalization versus a manual method of normalization. In particular, the manual normalization protocol introduces variability based on steps that require manual pipetting or other user inventions (such as calculations of dilutions). In contrast, the present bead-based method avoids a number of user touchpoints, which can both improve consistency and allow for greater automation of the method.

Table 4 provides a set of steps for manual normalization of 16 samples, and Table 5 provides a summary of the manual normalization steps used. As shown in Table 5, a standard manual normalization protocol requires 60 minutes of hands-on time, specialized equipment (such as a DNA quantification device), and the time for normalization scales with the number of samples.

TABLE 4
Manual normalization protocol for 16 samples

| Manual | | |
|---|---|---|
| Normalization | | |
| Step | Description | Time (min) |
| Prepare Mastermix | Prepare Qubit mastermix | 3 |
| Prepare | Add DNA into Qubit mastermix | 5 |
| Measurement | | |
| Measure | Perform Qubit | 5 |
| | quants on samples | |
| | Calculate dilution ratio | 5 |
| Dilute | Dilute sample down to concentration | 20 |
| | specified in workflow (<100 pg/μL) | |
| | Add diluted DNA into Qubit | 5 |
| | mastermix, triplicate | |
| Measure in | Perform triplicate Qubits | 20 |
| Triplicate | quants on sample to verify | |
| | within specific range | |
| Transfer | Transfer between 1.5- | 10 |
| | 2.5 uL to final | |
| | workflow depending | |
| | on triplicate quant | |
| Repeat | If triplicate measurements | 40 |
| (if needed) | are outside of range, repeat | |
| | dilution and remeasure in | |
| | triplicate. | |
| Total | Assuming no repeat sample preps. | ~60 minutes |
| | | per 16 |
| | | samples |

TABLE 5
Summary of manual normalization

| | |
|---|---|
| Hands-On Time | 60 minutes |
| Hands-Off Time | 0 minutes |
| Touch points | ≥8 |
| Quantification | Yes |
| Needed? | |
| Equipment Needed | Qubit or other DNA quantification |
| | device, pipettes |
| Does time scale with | Yes |
| sample number? | |

Table 6 provides a set of steps for bead-based normalization of 16 samples with NaOH-treated beads, and Table 7 provides a summary of the bead-based normalization steps used. As shown in Table 7, a bead-based normalization requires 60 minutes but only 20 minutes of hands-on time, and there is minimal time scale-up with an increase in the number of samples.

TABLE 6
Bead-based normalization protocol for 16 samples

| Bead Normalization | | |
|---|---|---|
| Step | Description | Time (min) |
| Prepare | Combine normalization | 2-3 |
| Mastermix | components | |
| Combine with DNA | Combine DNA and mastermix | 2-3 |
| Incubate on | Shake at 1800 rpm | 30 |
| Bioshake | for 30 minutes | |
| Wash | Wash excess DNA and | 10 |
| | clean with ethanol | |
| Elute | Elute in RSB | 10 |
| Transfer | Transfer 2 uL to | 5 |
| | final workflow | |
| Total | | ~60 minutes |
| | | for 16 samples |

TABLE 7
Summary of bead-based normalization

| | |
|---|---|
| Hands-On Time | 20 minutes |
| Hands-Off Time | 40 minutes |
| Touch points | 7 |
| Quantification | No |
| Needed? | |
| Equipment Needed | Bioshaker or device for |
| | mixing, pipettes |
| Does time scale with | Minimally |
| sample number? | |

Results using the bead-based protocol showed a significant decrease in coefficient of variance (CV) of final content compared to input DNA content after normalization. In this experiment, DNA input was only at or above predicted saturation. While the initial DNA had a CV of approximately 50%, the output DNA had a CV of approximately 12% (FIG. 24). These results indicate a robust normalization of the sample is seen using the present protocol with treated cellulose-coated beads.

Further, the size of library fragments was assessed using PCR after normalization. As shown in FIG. 25, the fragment sizes were similar whether manual normalization (i.e., manual control) or the present method with treated cellulose-coated beads was used. These results indicate that the bead-based normalization can produce libraries with similar fragment size as manual normalization.

A head-to-head evaluation was done using either the present bead-based normalization or manual normalization, and the results were compared to determine utility and user dependance. Data from samples prepared by the present normalization with treated beads were compared to samples using manual normalization (FIG. 28) with generally similar results.

In summary, the present data show robust normalization of library fragments using NaOH-treated cellulose-coated beads.

### Example 7. Evaluation of Parameters for Normalization with Untreated Cellulose Beads

Library normalization with untreated cellulose-coated beads was also evaluated. The normalization method was the same for the treated and untreated cellulose-coated beads, with a representative protocol for performing normalization with untreated cellulose-coated beads shown in FIG. 27.

When compared to NaOH-treated cellulose-coated beads, untreated cellulose-coated beads showed significantly less capture of DNA (FIG. 29, showing a representative 80%-120% lower binding capacity of untreated beads versus treated beads). HTC beads served as a comparator type of untreated cellulose bead that also showed significantly lower DNA capture than the present NaOH-treated cellulose-coated beads. While HTC beads showed an increase in DNA binding after NaOH treatment, overall yield was higher with the present cellulose-coated beads (data not shown).

This decreased capture could be offset by doubling the concentration of untreated beads in relation to the treated beads. Since the number of beads used for normalization may be very small (for example if the DNA libraries to be normalized have a small amount of DNA), performing a normalization method with a relatively greater number of untreated beads can help the user visualize the bead pellet during a normalization method. With visualization of the bead pellet, wash steps can be easier to perform.

Experiments on different wash protocols indicated that 2 washes decreased DNA binding to beads and allowed for the normalization yield to align within a “target zone” for producing the best sequencing data (FIG. 30). Alternatively, a single high-volume wash (such as a 200 μL wash in a 96-well plate) can improve results as compared to smaller-volume washes (FIG. 31). These data on washes suggest that carryover of excess DNA (i.e., DNA not bound to untreated beads) is the cause of undesired excess library yield after normalization. Two washes with wash buffer (or alternatively a single high-volume wash) may be included in a standard normalization protocol using untreated beads to reduce this carryover of excess DNA. Such increased washing measures may be especially relevant when normalizing a greater amount of DNA library.

Experiments with normalization using untreated beads showed that control of pipetting volumes is important. For example, FIG. 32 shows that uncontrolled volume changes can alter the yield from normalization. Accordingly, a user will likely want to evaluate conditions and then avoid volume changes from that protocol.

The effect of the percentage of PEG comprised in the binding buffer was also evaluated for normalization using untreated beads. For example, FIG. 33 shows that increasing the percentage of PEG300 in a binding buffer increases the amount of DNA captured. These data indicate that a user could evaluate normalization with different bead lots, wherein the different bead lots may have intrinsically different binding capacities, by modifying the PEG composition of the binding buffer. Such ability to tune the normalization with untreated based on the composition of the binding buffer is an advantage to allow for consistent results with different batches of cellulose-coated beads.

Similarly, the type of PEG included in the binding buffer influences normalization results with untreated beads, with binding buffer comprising PEG8000 showing much greater DNA capture than binding buffer comprising PEG300 or PEG400 (FIG. 34). Thus, while PEG8000 is commonly comprised in buffers for bead capture, the large amount of DNA capture may be undesired for bead-based normalization with cellulose-coated beads.

With the present steps of binding buffer, washing, and other conditions, a generally linear association of DNA capture to bead amount was seen (FIGS. 35A and 35B). Thus, users could estimate an appropriate number of untreated beads to use for a desired level of capture during normalization once they have evaluated their conditions.

Further, when a bioshaker is used, 15 minutes of incubation/mixing was sufficient for binding of DNA to untreated beads (FIG. 36). In other words, the reaction kinetics for binding of DNA to untreated beads with a bioshaker was high, and long incubations were not necessary. When manual mixing was used, manual mixing for up to 30 minutes did not achieve levels seen with bioshaker mixing for samples with a large DNA input (for example 100 ng). Accordingly, users may choose to use a bioshaker and to limit mixing to 15 minutes, thus shortening the time for the normalization protocol.

Results showed sufficient DNA capture for a range of different DNA sources with sizes ranging from 25 kb-60 kb (FIG. 37). Thus a variety of different DNA sources can be used with the present normalization methods with untreated cellulose-coated beads. Further, elution of DNA from untreated beads into resuspension buffer was very efficient, and an incubation of 1-5 minutes is sufficient for elution (FIG. 38). This efficient elution can allow for shortened protocol times.

### Example 8. Protocol for Bead-Based Normalization with Untreated Cellulose-Coated Beads

Normalization protocols can be developed with untreated cellulose-coated beads (see, for example, FIG. 27).

A first step is preparation of a stock of 50× diluted beads. Untreated cellulose-coated beads are mixed well and then 20 μL of beads is added to 980 μL of a 20% PEG300 solution. The mixture is vortexed, and these 50× diluted beads are used to prepare a mastermix according to Table 8.

TABLE 8
Preparation of mastermix comprising beads

| | μL to combine | μL to combine for |
|---|---|---|
| Prepare Mastermix | for 1 sample | 16 samples |
| Binding Buffer | 5.5 | 88 |
| 10X Diluted Bead | 5.5 | 88 |
| Total (with | 11 | 176 |
| loverage) | | |

After preparing the mastermix, 10 μL of DNA (containing at least 10 ng of DNA) is added to a well of a plate (such as a PCR plate). The mastermix is vortexed until homogeneous, and then 10 μL of prepared mastermix is immediately added to each well containing 10 μL of DNA. The plate is sealed with a microseal and put on a bioshaker for up to 30 minutes at 1800 rpm or alternatively for a shorter incubation time of 15 minutes.

The plate is removed from the bioshaker and placed on a plate magnet for 1 minute. The supernatant is carefully removed while taking care not to disturb the bead pellet. This step is potentially easier with untreated beads (in comparison to NaOH-treated beads) as the number of beads would be higher as each bead captures less DNA, and the user will be better able to visualize the bead pellet.

The plate is removed from the plate magnet, and 150 μL of wash buffer (i.e., wash buffer 1) is added immediately to each well. Using a p200 pipette set to 140 μL, each well is mixed at least 10 times. The plate is then placed on a plate magnet, and this wash step is then repeated. Alternatively, a single high-volume wash (e.g., a single 200 μL wash) may be performed.

The supernatant is gently removed, taking care not to disturb the beads. The plate is removed from the plate magnet, and 150 μL of 80% EtOH (i.e., wash buffer 2) is immediately added to each well. Using a p200 pipette set to 140 μL, each well is mixed at least 10 times. The plate is then placed on a plate magnet for 1 minute, and the supernatant is gently removed, taking care not to disturb the beads. The plate is briefly spun down, and a p20 pipetter is used to remove excess liquid. The plate is allowed to dry for 2 minutes.

Then, 80 μL of RSB is added to each sample well. Using a pipette set to 50 μL, each sample is mixed at least 10 times. The plate is allowed to incubate for 5-10 minutes and placed on a plate magnet for 1 minute. A total of 70 μL of supernatant is pipetted into a new well for downstream steps (such as sequencing) with the normalized library.

### Example 9. Comparison of Normalization with Treated and Untreated Beads

Experiments were performed to directly compare normalization of the same library with either untreated cellulose-coated beads or NaOH-treated cellulose-coated beads. While both types of beads can normalize libraries, results were evaluated to determine whether the different approaches impacted the normalized library.

For these experiments, normalization was performed after library preparation and before PCR amplification. This approach may be preferred when libraries comprise relatively large fragments (e.g., 6000-8000 base pair fragments for long-read sequencing). For libraries comprising relatively small fragments (e.g., 300-600 base pairs fragments for short read sequencing), normalization may be performed after library preparation and PCR amplification.

As shown in FIG. 39, the fragment size of the same library before any normalization (i.e., “raw library”) or after normalization with treated or untreated beads was evaluated. Fragment size was determined using a Bioanalyzer.

Two different libraries were evaluated, one library had a smaller average fragment size of 6330 base pairs without normalization and other library had a larger average fragment size of 8030 base pairs without normalization. Normalization with NaOH-treated beads yielded normalized libraries with average fragments sizes that were similar or smaller to the raw library. In contrast, the average fragment size of the normalized library with untreated beads was larger than the raw library.

These results indicate that normalization with NaOH-treated cellulose-coated beads may have relatively little impact on fragment size, with little change in the fragment size of the normalized library versus the raw library. In contrast, normalization with untreated cellulose-coated beads may shift the library to a larger maximum peak size and size distribution. For example, the data in FIG. 39 indicate that normalization with untreated cellulose-coated beads after PCR amplification resulted in a normalized library with fragments sizes that are approximately 5%-29% larger than the raw library.

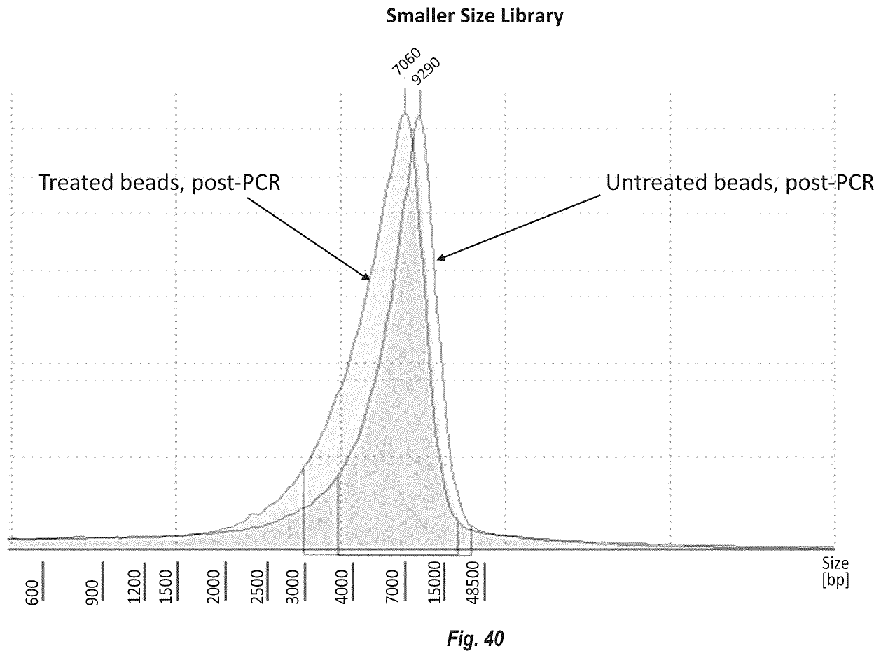

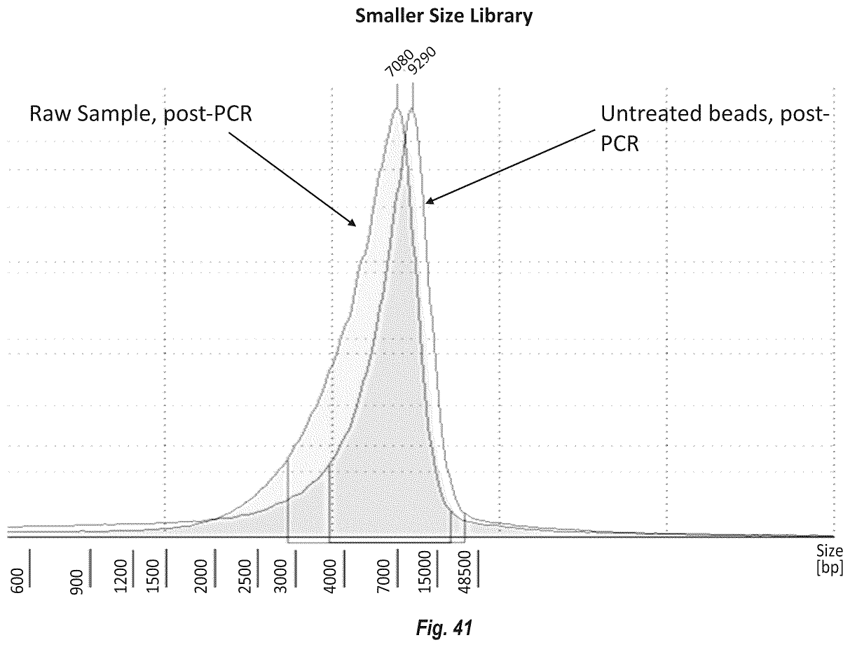

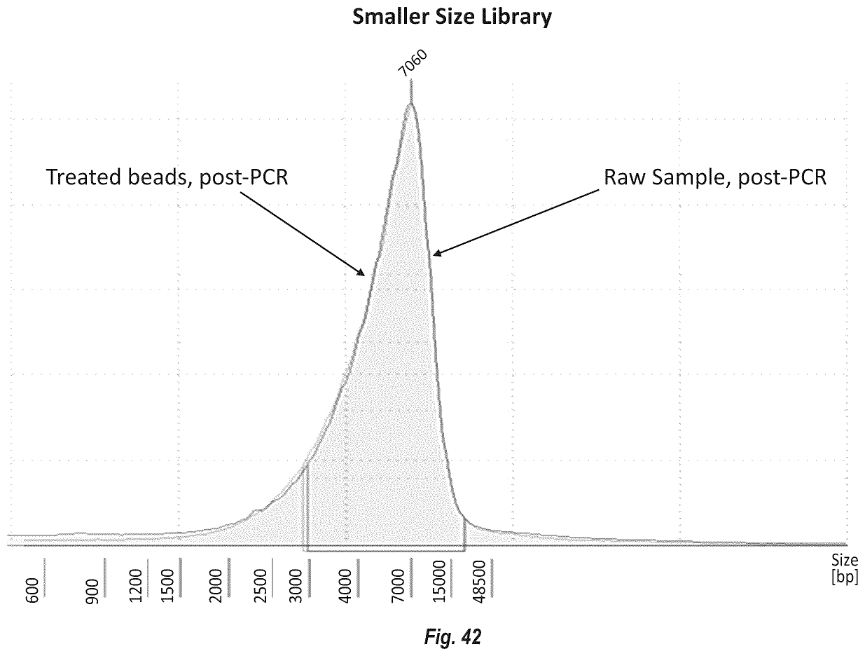

FIGS. 40-42 provide additional data on normalized library sizes after PCR amplification for the library with relatively smaller size (with raw library fragments of approximately 7060 base pairs after amplification). Normalized library fragment size after PCR amplification was larger for normalization with untreated beads versus normalization with NaOH-treated beads (FIG. 40). In addition, the fragment size was larger after normalization with untreated beads in comparison to the raw library (FIG. 41). In contrast, the fragment size of the library after normalization with NaOH-treated beads was the same as the raw library (FIG. 42).

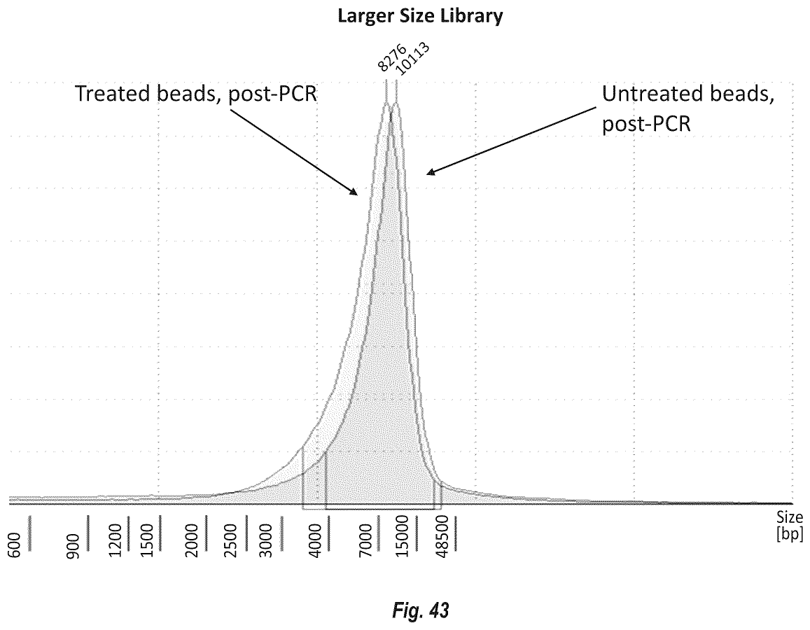

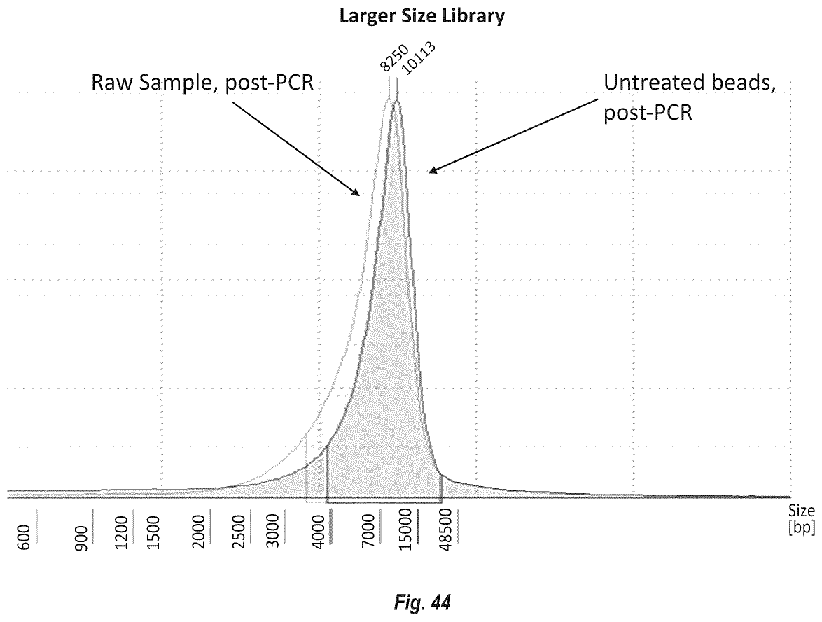

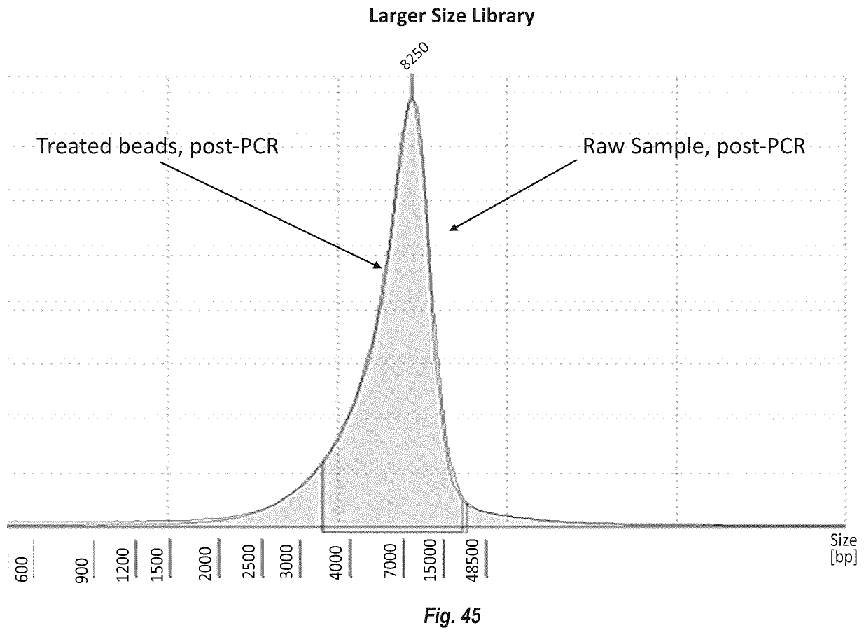

FIGS. 43-45 provide additional data on normalized library sizes after PCR amplification for the library with relatively larger size (with raw library fragments of approximately 8250 base pairs after amplification). Normalized library fragment size after PCR amplification was larger for normalization with untreated beads versus normalization with NaOH-treated beads (FIG. 43). Further, the fragment size was larger after normalization with untreated beads in comparison to the raw library (FIG. 44). In contrast, the fragment size after PCR amplification of the library after normalization with NaOH-treated beads was the same as the raw library (FIG. 45).

These results indicate that normalization with NaOH-treated cellulose-coated beads may have minimal effect on fragment size, while normalization with untreated cellulose-coated beads may lead to a normalized library comprising larger library fragments than the raw library.

This ability to control fragment size of the library may be preferred for different methods. If a user prefers to have a smaller fragment size or to avoid altering the fragment size during normalization, they may prefer to use NaOH-treated beads. For example, there may be libraries wherein longer fragment sizes are detrimental to sequencing results, such as causing less effective read 2 sequencing.

For other methods, such as long-read sequencing methods, the user may prefer to use untreated beads and generate a normalized library with generally larger-sized fragments. The larger average fragment size may improve the ability to perform de novo sequencing by preferentially capturing sequencing data from longer fragments. A size shift to larger fragment size of the normalized library can thus provide an additional benefit for the sequencing of longer inserts. Also, untreated beads may be easier to work with for normalization because they require more beads than NaOH-treated beads, resulting in a pellet that is visible and easier to handle.

Accordingly, the user can choose normalization with either treated or untreated cellulose-coated beads based on their experimental objectives and the downstream sequencing platform used.

### Example 10: Evaluation of GC Bias for Bead-Based Normalization

While approximately 5% of genome sequences have GC bias of 62% or greater, these sequences have not been well-characterized and generally show low coverage.

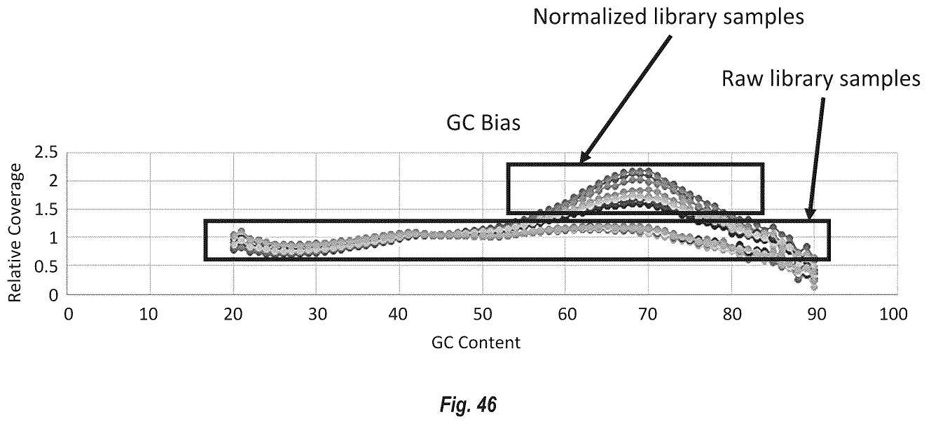

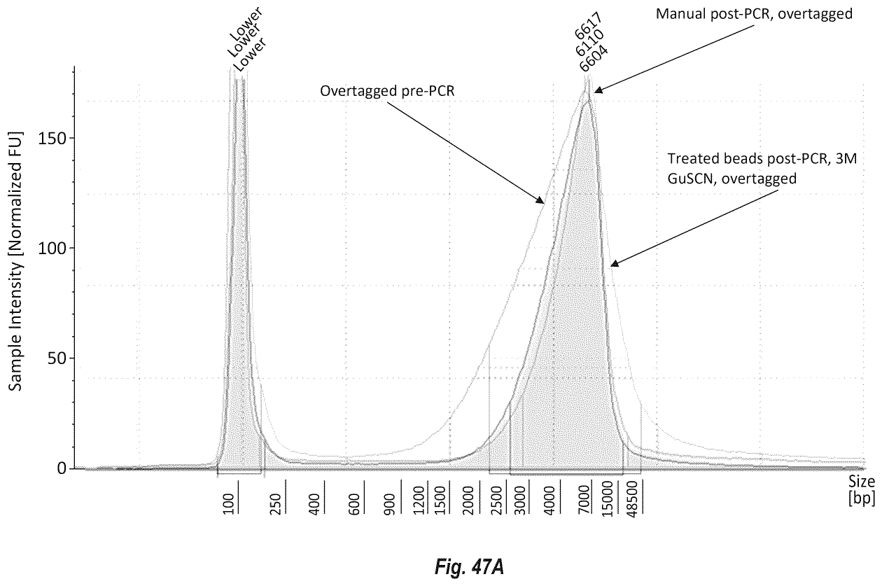

As shown in FIG. 46, normalization with untreated or NaOH-treated beads unexpectedly showed a GC bias with relatively high coverage of samples with 60%-80% GC content. In contrast, the same raw library (i.e., unnormalized) was not GC-biased and had a relatively flat slope of coverage across GC content. These data indicated that normalization with cellulose-coated beads may induce GC bias in samples and could be used to exploit analysis of such high-GC regions. Further experiments were performed to better characterize this GC bias.

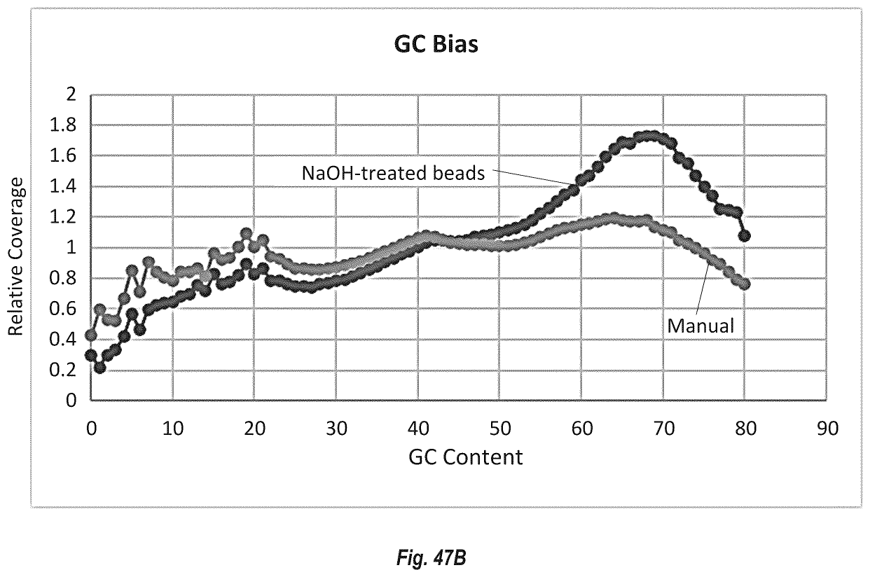

FIGS. 47A and 47B show results for normalization with NaOH-treated beads and a binding buffer comprising 3M GuSCN. These results showed high overlap with manually normalized fragments for fragment size after PCR, but GC bias was higher for the library normalized with NaOH-treated beads in comparison to the library manually normalized. Thus, the present normalization with cellulose-coated beads can allow for greater sequencing coverage of fragments with 62% or greater GC content.

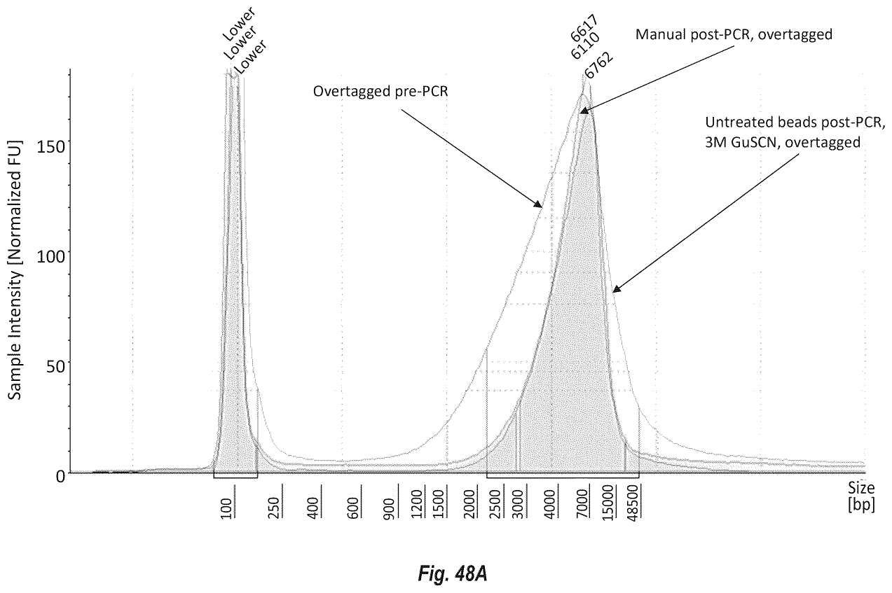

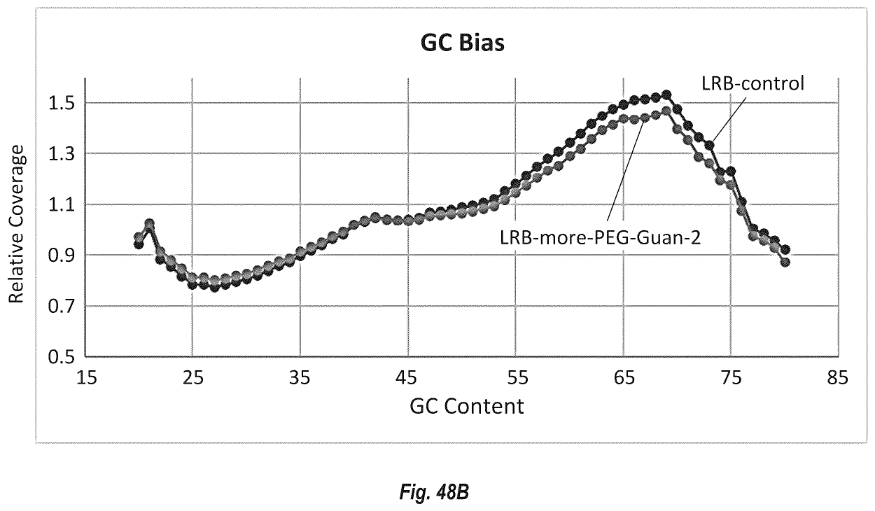

FIG. 48A shows results for normalization with untreated cellulose-coated beads and a binding buffer comprising 3M GuSCN. These results showed high overlap with manually normalized fragments for fragment size after PCR, indicating that the higher GuSCN content led to library fragment sizes more similar to manual normalization. The GC bias was similar for the library normalized with NaOH-treated beads in comparison to the library normalized with untreated beads (FIG. 48B). Thus, the present normalization with cellulose-coated beads (both untreated and NaOH-treated) allows for greater sequencing coverage of fragments with 62% or greater GC content.

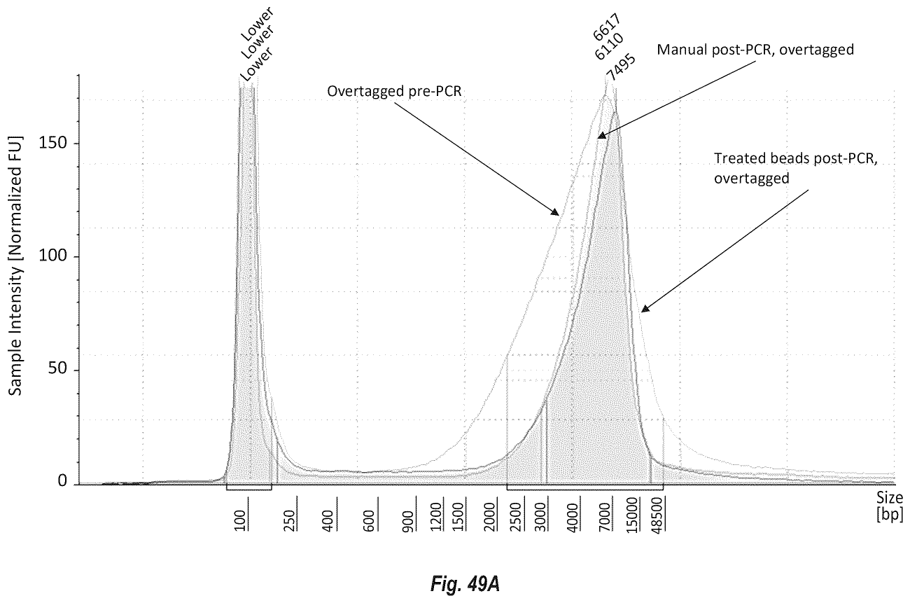

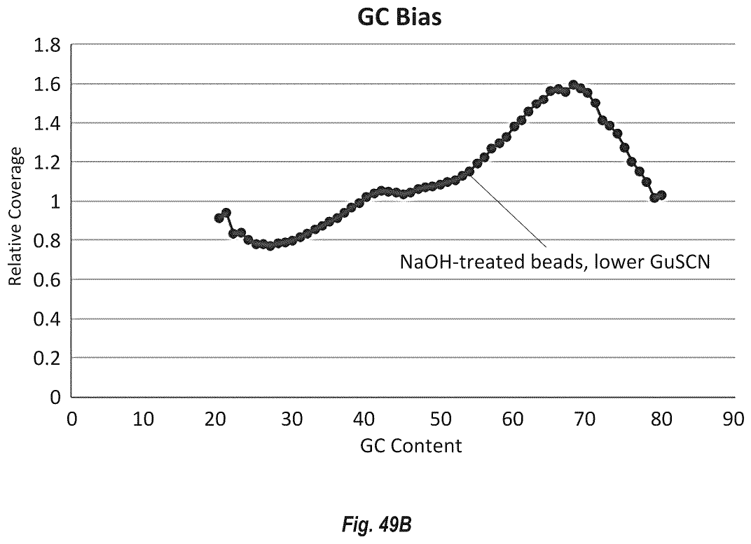

FIG. 49A shows that high overlap of the library normalized with NaOH-treated beads and standard binding buffer (less than 3M GuSCN) with the manually normalized library. FIG. 49B shows that GC bias was maintained in these conditions.

Thus, both untreated and NaOH-treated cellulose-coated beads showed GC bias that was maintained in different binding buffers.

The impact of PEG content of the binding buffer on GC bias was also evaluated. FIG. 50 shows a range of different PEG8000 percentages (weight/volume) and the impact on the resulting library and sequencing results. Results are presented for both a control library (with fragment size of approximately 8 Kb) and an overtagged library (with fragment size of approximately 6 Kb). These results compared results from a library comprising long fragments compared to a library with relatively shorter libraries and how both libraries react to different PEG8000 content. While fragment size was not affected by PEG8000 content for either library type, DNA capture increased with higher PEG8000 content in the binding buffer. In contrast, GC bias decreased with higher PEG8000 content in the binding buffer for both libraries.

These results indicate that a user could tune the normalization to desired conditions. For example, if the user prefers to have more GC bias (i.e., sequence more fragments with relatively high GC content), they could use a buffer with a lower PEG8000 content for normalization. If instead a user wanted to maximize library yield with increasing GC bias, they could use a binding buffer with PEG8000 content of 7.5% or greater.

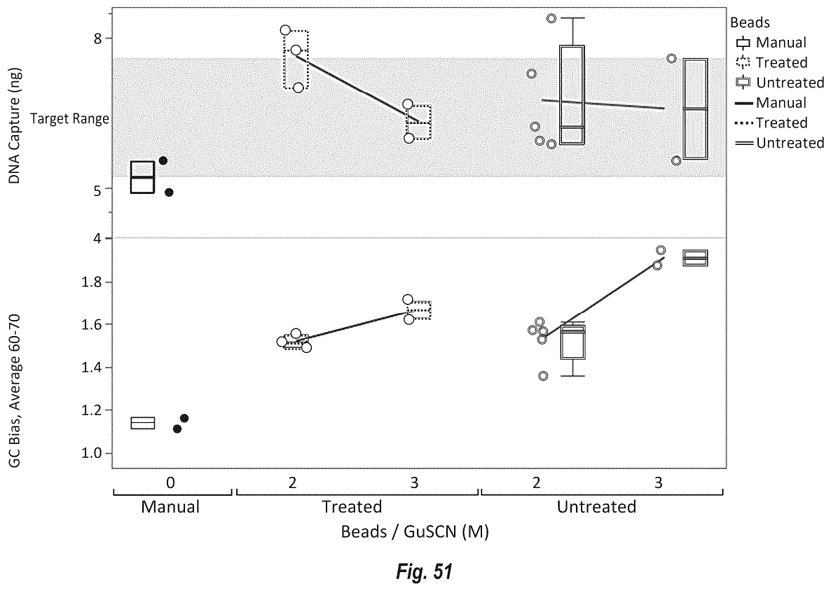

Higher GuSCN content of the binding buffer could also increase GC bias of the normalized library with either untreated or NaOH-treated beads (FIG. 51). The GuSCN concentration did not significantly impact DNA capture.

Results with varying PEG300 content in the binding buffer for normalization paralleled those with varying PEG8000 content (FIG. 52). PEG300 content in the binding buffer had a positive correlation with DNA capture and a negative correlation with GC bias. Most GC bias differences were seen from 10%-30% PEG300 conditions with much less being seen between 30-35% PEG300. Similar results were seen with untreated and NaOH-treated cellulose-coated beads.

Based on these results, a user could choose their preferred PEG (such as PEG300) and whether or not they wanted to induce GC bias and prepare a binding buffer for bead-based normalization with untreated or NaOH-treated cellulose-coated beads accordingly.

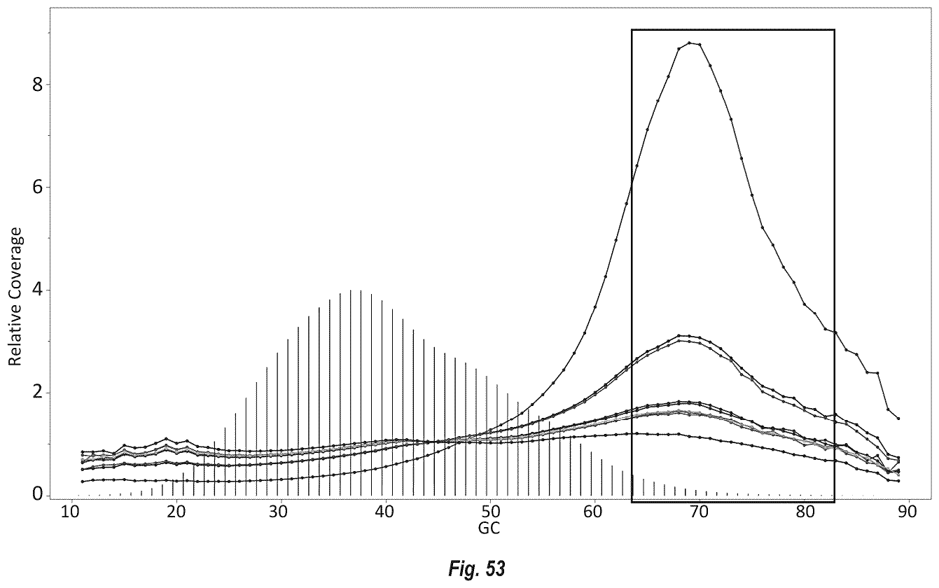

FIG. 53 shows a summary of results from a variety of conditions for normalization with cellulose-coated beads. These results indicate a consistent increase in coverage of GC-heavy regions in bead-normalized libraries as compared to manually normalized libraries, which is likely in part to the low coverage normally seen for these GC-heavy sequences using manual normalization.

In sum, data on the normalization with cellulose-coated beads consistently showed the ability to increase coverage of GC-heavy regions.

### Example 11: Comparison of Binding Characteristics of Various Bead Types

A variety of different beads with DNA-binding characteristics were tested to see if results with cellulose-coated beads were generalizable.

FIG. 55 shows results with carboxylated magnetic beads (which may be referred to in the literature as SPRI beads or ITB beads). The carboxylated beads were mixed with silica magnetic beads to increase the bead amount for better visualization of the bead pellet. The ratio was 1:24 for carboxylated beads to silica beads. A range of 50, 200, 500, and 900 ng library input was tested. Across a range of DNA inputs, a consistent 12% recovery rate of the DNA was seen. The results showed a linear relationship between yield (i.e., DNA output) and DNA input, without ever reaching plateau. Thus, the mixture with carboxylated beads is not appropriate for input titration and normalization, because the output is a function of the DNA input without allowing for a plateau of yield at a desired level.

Next, silica magnetic beads were evaluated using Apostle and AMO beads and a library input of 50, 200, 500, 750, or 1000 ng. As shown in FIG. 56, DNA binding to 10 μL AMO beads or Apostle beads plateaued, but the DNA yield was much higher than the desired target output range of 60 ng of DNA. Further, diluting the AMO beads or Apostle beads (to 2.5 μL for Apostle beads and 5 μL for AMO beads) produced a bead pellet that was still easily workable, but the plateau for DNA binding was still above the desired upper limit of 60 ng. For example, the DNA yield was approximately 70 ng for diluted Apostle beads and 93 ng for diluted AMO beads. Thus, with their high DNA-binding capacity and small size, the silica magnetic beads were not a good choice for normalizing a library.

The presently described cellulose-coated beads were used for normalization of cfDNA, as shown in FIGS. 57A and 57B. The relatively large size (approximately 10 μm) and low DNA-binding capacity allowed for a yield of about 65% with a plateau within the target range (37 ng in the representative example in FIG. 57A). Further, the insert size was in the desired range (FIG. 57B). Thus, cellulose-coated beads are an optimal bead type for normalizing cfDNA.

### Example 12: Automated Protocol for Bead-Based Normalization for Extracted ctDNA

Evaluation of circulating tumor DNA (ctDNA) is important for a number of cancer diagnostics, but normalization before library preparation is critical due to inherent sample variation. The cellulose-coated beads described herein can be used for a bead-based normalization (BBN) protocol for extracted ctDNA or other types of cfDNA.

The basic steps of a BBN protocol are (1) cfDNA binding, (2) washing, and (3) elution. The cfDNA binding can be performed with 150 μl cfDNA plus 150 μl binding buffer. Then, 5 μl (1:10 diluted) beads at room temperature can be added and incubated for 20 minutes. After the incubation, the beads can be washed with 250 μl wash buffer 1, and then with 250 μl 80% ethanol with air drying for 5 minutes. The normalized ctDNA can then be eluted with 50 μl RSB, with 15-minute mixing and 10-minute shaking.

Table 9 below shows a representative protocol with a turnaround time (TAT) of approximately 1 hour for 8 samples.

TABLE 9
Steps of protocol for normalization of cfDNA

| | |
|---|---|
| Step | Starting material: 4 ml of plasma |
| Extraction Elution | 150 μL of cfDNA |
| Add diluted bead | Adding 5 μL 1:10 diluted cellulose-coated beads |
| Transfer Binding Solution | 150 μL Binding Buffer |
| Digestion incubation | Mix reaction and incubate at RT for 20 min. |
| Magnet Incubation | Transfer plate to magnet. Incubate for 3 min. |
| Remove supernatant | Remove supernatant and dispense in liquid waste container. |
| Transfer Wash 1 | 250 μL Wash 1 |
| Magnet Incubation | Transfer plate to magnet. Incubate for 3 min. |
| Remove Wash 1 | Remove Wash 1 and dispense in liquid waste container. |
| Transfer Wash 2 | 250 μL EtOH (80%) |
| Magnet Incubation | Transfer plate to magnet. Incubate for 2 min. |
| Remove Wash 2 | Remove Wash 2 and dispense in liquid waste container. |
| Airdry | Airdry at RT for 5 min. |
| Transfer resuspension buffer | Add 50 μL RSB to plate and pipetting mix for 15 cycles. |
| (RSB) | |
| Elution incubation | Incubate off magnet with shaking (1800 rpm) for 10 min. |
| Magnet incubation | Transfer plate to magnet. Incubate for 2 min. |
| Transfer cfDNA | Transfer 45 μL supernatant to new plate “cfDNA” plate. |

Results with automated BBN are shown in FIGS. 58A-58C, which summarize the desired yield (FIG. 58A) and fragment size (FIG. 58B) are maintained with the automated process.

FIG. 58C shows the impact of cfDNA extraction in a model of high molecular-weight DNA impurities, which can commonly be found in plasma. The first two lanes in FIG. 58C, B1 and C1, are duplicate gel runs of a control sample made by spiking in high molecular-weight DNA as “impurities.” High molecular-weight DNA is a known impurity type always present in plasma samples that often is hard to eliminate and that may interfere with downstream cfDNA quantitation using a DNA intercalation dye. High molecular-weight DNA spike-ins were performed to generate a final cfDNA sample that contains 55% cfDNA (as shown in FIG. 58C) with a significant amount of high molecular-weight DNA (shown in circles for samples B1 and C1).

While the first two lanes of FIG. 58C (B1 and C1 samples) did not see any extraction, the rest of 8 lanes (8 repeats in separate wells) are samples subjected to extraction with cellulose-coated beads described herein, and their resulting cfDNA % all increased based on removal of most high molecular-weight DNA “impurities”. This is a distinct unique feature of the present cellulose-coated bead extraction, when comparing to other extraction products on the market such as MagMax™ from Thermo Fisher, NeoGeneStar™ from NeoGeneStar, and QiaSymphony® extraction from Qiagen, to name a few (data not shown).

MagMax™, NeoGeneStar™, and QiaSymphony® all require quantitation step for manual cfDNA “normalization” in their corresponding workflows, and high molecular-weight DNA impurities may falsely interfere with this manual quantitation and result in over-dilution of cfDNA samples for downstream library preparation. In contrast, the present cellulose-coated beads can remove the high molecular-weight DNA impurities, which allows for more accurate manual quantitation cfDNA normalization in addition to allowing for automated normalization, if desired.

This feature of removing high molecular-weight DNA impurities during extraction can improve certain clinical products/processes. For example, a clinical provider can incorporate extraction with cellulose-coated beads into a protocol to replace their existing extraction modality (such as MagMax™, NeoGeneStar™, or QiaSymphony®) while keeping other workflows unchanged. In this way, the improved extraction with cellulose-coated beads with reduction of high molecular-weight DNA can be incorporated into an existing clinical production process. Thus, a clinical workflow with a step of manual normalization can be maintained (without requiring any hardware upgrade for automation) while allowing for the benefits of cfDNA extraction with cellulose-coated bead to remove high molecular-weight impurities.

Thus, present cellulose-coated beads may have distinct advantages for cfDNA extraction as compared to silica-based beads, such as MagMax™, NeoGeneStar™, and QiaSymphony®. This benefit can be incorporated together with an automated BBN protocol, as described in Table 9, but the advantages of extraction with cellulose-coated beads can also be seen with manual normalization protocols.

### Example 13: Combined Purification, Size Selection, and Normalization

In order to prepare and normalize a shotgun library, such as a ctDNA library, (1) purification, (2) size selection, and (3) concentration normalization must all be performed before sequencing. FIG. 59 summarizes that concentration normalization allows for more robust sequencing across a range of samples.

FIG. 60 at the top arrows (process 1) shows a standard workflow for preparing a ctDNA library, with purification and size selection with SPRI beads followed by a separate normalization step. Such normalization after purification and size selection can be performed with streptavidin beads with pre-attached biotinylated DNA (library normalization beads, LNB1) together with appropriate buffers and washes (such as library normalization buffer (LNA1) and library normalization wash (LNW1)). A representative normalization protocol with LNB1 beads is described in Best practices for LNB1 handling in bead-based normalization for TruSight Oncology 500 workflows, Illumina, Aug. 22, 2022.

Current normalization workflows for shotgun libraries have a number of limitations. For example, streptavidin beads with pre-loaded DNA for use in normalization are expensive. Further, use of streptavidin beads with pre-loaded DNA may lead to challenging quality issues including over-normalization or under-normalization that may result in a user loading too much sample or too little sample onto sequencers, both of which can cause sequencing failures. Another failure type known to potentially occur with current methods of library normalization is non-specific binding, meaning streptavidin beads with pre-loaded DNA can non-specifically absorb undesired impurities during the normalization process, resulting in undesired/borderline sequencing results or even failures.

Any sequencing failure in a clinical environment is highly undesirable. For example, a patient's plasma samples may only be sufficient for one normalization/sequencing reaction. In this case, sample recollection may require a long time if there is a sequencing failure. In some cases, medical insurance companies may not reimburse or reimburse enough for additional re-sampling and re-sequencing, so any sequencing failure can have severe consequences for clinical providers and patients.

In summary, the current method with shotgun libraries uses SPRI beads for purification and size selection and then uses streptavidin magnetic beads, pre-attached with biotinylated single-stranded DNA that is complementary to shotgun libraries, for normalization. The normalization is performed with denaturation using 0.1 M NaOH to generate normalized library samples. Thus, current ctDNA library preparation protocols ask that the user perform normalization as a separate step with separate beads from the purification and size selection; however, these separate steps add user time and reagent costs to the workflow. Accordingly, current methods of size selection/purification followed by normalization are complex, require expensive bead materials, and are challenging to automate.

While carboxylated beads can be used for size selection, they do not work for normalization (due to their linear DNA-binding characteristics as shown in FIG. 55). This finding is summarized in process 3 of FIG. 60. Thus, while SPRI beads are useful for purification/size selection of libraries, normalization was not successful with this bead type.

In contrast, process 2 of FIG. 60 shows that the presently described cellulose-coated beads can be used for purification, size selection, and normalization of shotgun libraries with a single type of bead. This process can greatly simplify the steps between library preparation and sequencing. The benefits of using the present cellulose-coated beads are that the process is simpler, has lower cost, and is easy to automate with a significantly slower turnaround time. Thus, the presently described cellulose-coated beads allow for a protocol of purification of a library from reagents used to prepare the library, size selection, and normalization with a single type of beads.

FIG. 61A shows a representative cfDNA library yields with normalization across a range of input DNA amounts. Further, FIG. 61B shows similar post-normalization output peak fragment sizes for 50 ng-350 ng DNA inputs, indicating uniform normalization performance over a range of DNA inputs. Thus, the presently described cellulose-coated beads allow for normalization of small amounts of library inputs (such as 10 ng-50 ng), as well as larger amounts of library inputs.

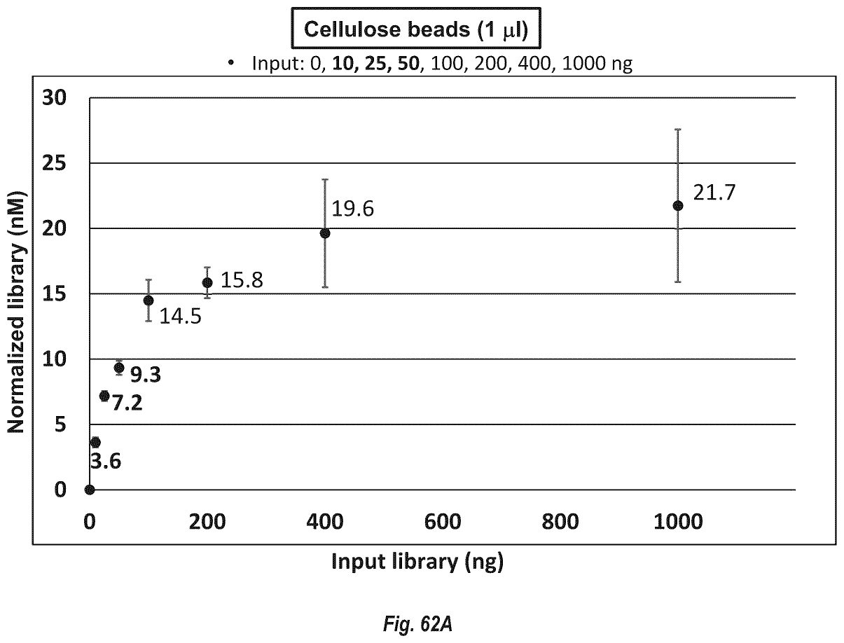

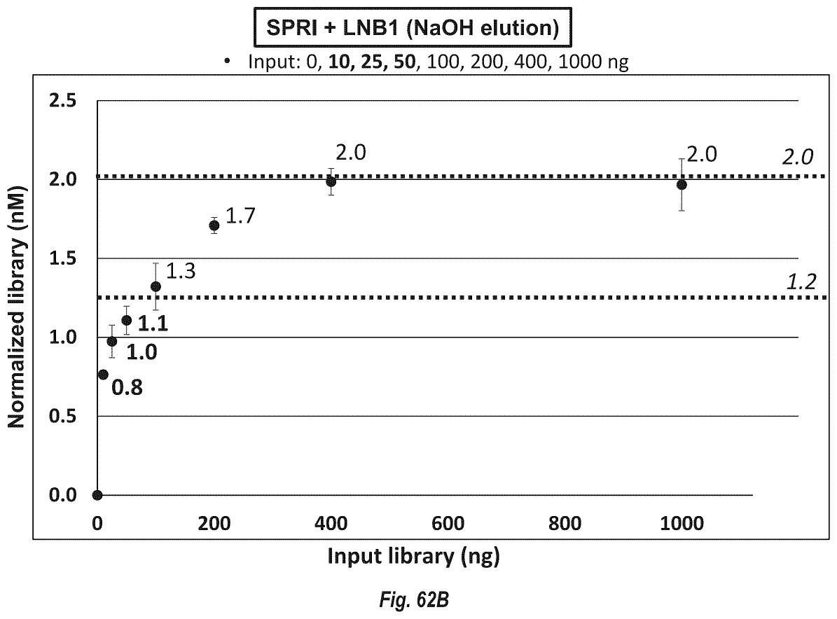

Purification/size selection and normalization with cellulose-coated beads (as shown in FIG. 62A) was compared to a 2-bead method of purification/size selection with SPRI beads followed by normalization with LNB1 beads (streptavidin magnetic beads with pre-attached biotinylated DNA, as shown in FIG. 62B). These data indicate that the significantly faster and easier protocol with cellulose-coated beads reached a plateau of normalized library in a similar fashion as the 2-bead method.

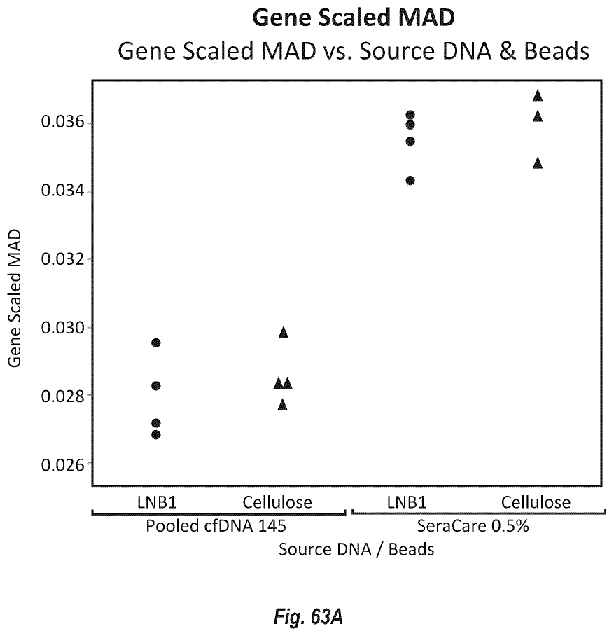

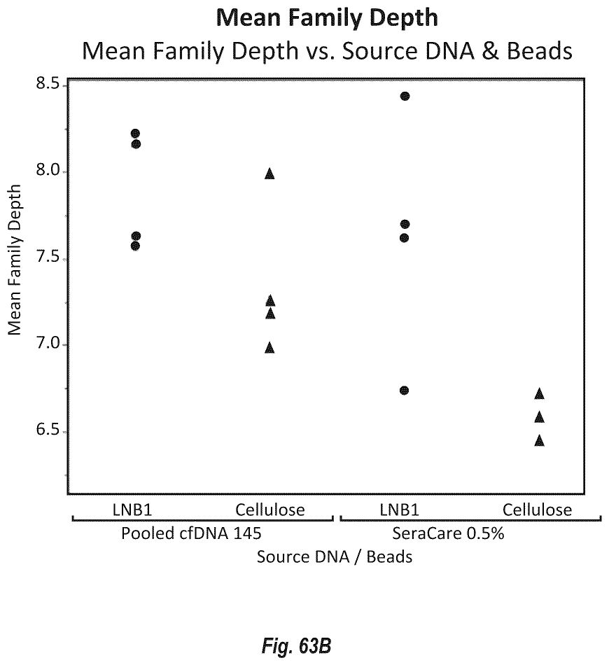

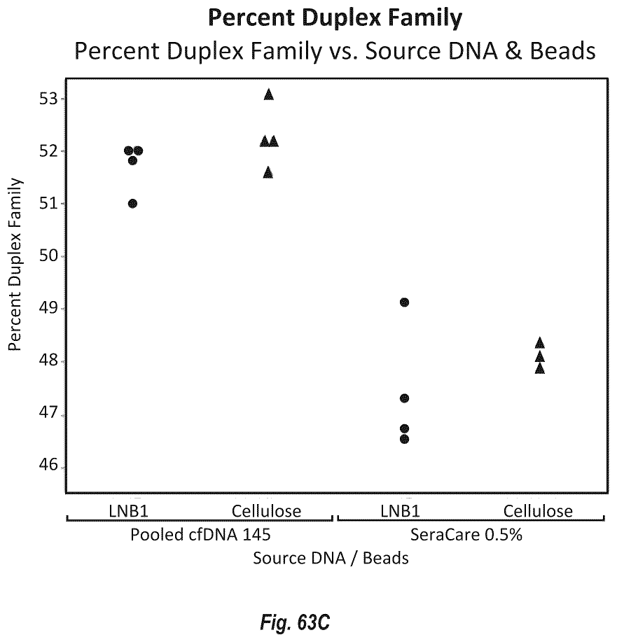

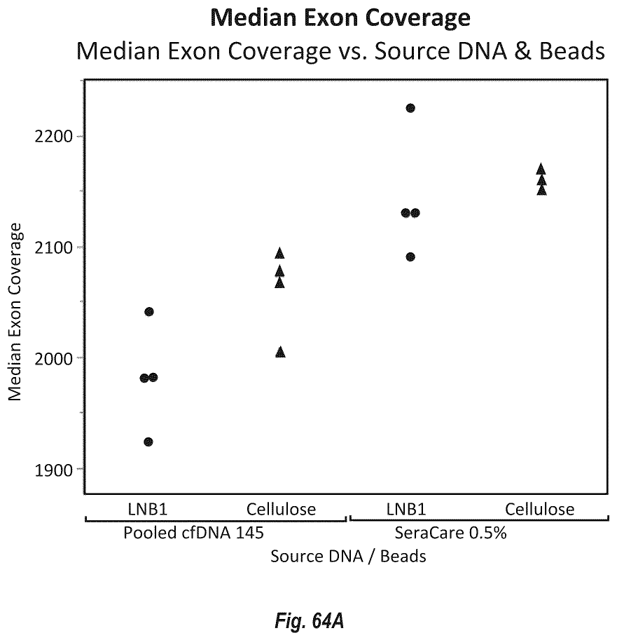

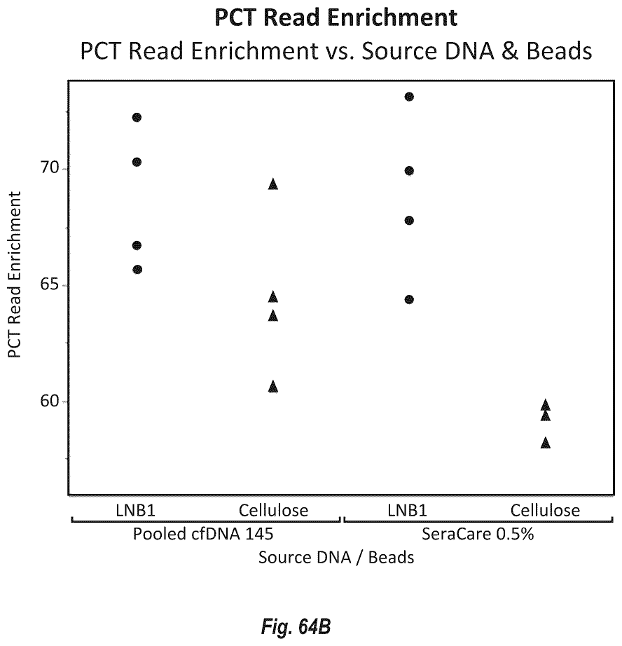

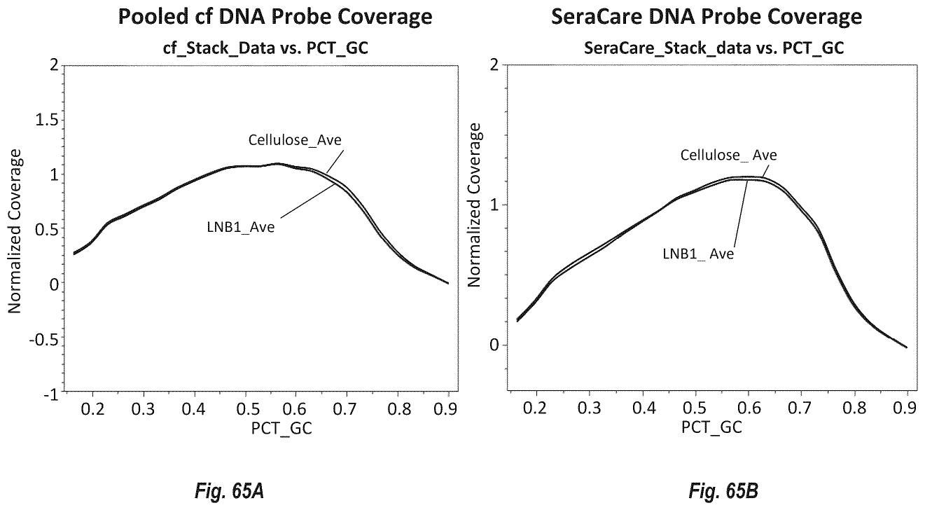

FIGS. 63A-63C shown that sequencing metrics were similar for purification/size selection and normalization with cellulose-coated beads as compared to a 2-bead method of purification/size selection with SPRI beads followed by normalization with LNB1 beads for gene scaled MAD (FIG. 63A), mean family depth (FIG. 63B), and percent duplex family measurements (FIG. 63C). In addition, median exon coverage (FIG. 64A) and PCT read enrichment (FIG. 64B) showed only minor differences before the method with cellulose-coated beads as compared to the 2-bead method of purification/size selection with SPRI beads followed by normalization with LNB1 beads. In addition, no GC bias difference was observed for sequencing data from cellulose-coated beads (FIG. 65A) as compared to the 2-bead method of purification/size selection with SPRI beads followed by normalization with LNB1 beads (FIG. 65B).

Table 10 provides brief descriptions of the different metrics and materials used to characterize sequencing data with different normalization protocols.

TABLE 10
Sequencing metrics and experimental materials

| | |
|---|---|
| Gene Scaled MAD | Median of absolute deviations (MAD) from the median |
| | of the normalized count, following a correction for the |
| | median normalized count for each gene of each copy |
| | number variation (CNV) target region after excluding |
| | genes with a potential deletion. This metric is a measure |
| | of the variability of a sample. |
| Mean Family Depth | Family means a group of reads that originate from copies |
| (Count) | of the same DNA sequence. Mean family depth is the |
| | average number of paired reads across all families before |
| | read collapsing. This metric evaluates PCR duplication |
| | level of libraries. |
| Percent Duplex Family | The percentage of read families that are duplex families, |
| (%) | meaning reads from both sense and antisense strands. |
| | Duplex family helps error correction of the analysis. |
| Median Exon Coverage | Median exon read coverage across all exon bases. |
| (Count) | |
| PCT Read Enrichment | Percentage of all aligned reads that are aligned to the |
| (%) | enrichment target. |
| Probe Coverage | Normalized probe coverage across all probes with |
| | various GC content. This metric is used to evaluate any |
| | relative sequencing GC bias between different |
| | normalization methods. |
| Pooled cfDNA 145 | cfDNA extracted from human plasma pool designated |
| | #145. |
| SeraCare 0.5% | Standard purchased from SeraCare Life Sciences |
| | (material number 0710-0531, Seraseq ctDNA Complete |
| | Mutation Mix AF 0.5%). This ctDNA standard contains |
| | 0.5% allelic fraction mutation mix. |

Thus, purification/size selection and normalization with cellulose-coated beads (process 2 of FIG. 60) showed similar sequencing results in comparison to a 2-bead method of purification/size selection with SPRI beads followed by normalization with LNB1 beads (process 1 of FIG. 60, which is a current method). However, the method with cellulose-coated beads has advantages in quicker turnaround, ease of use, and cost-saving since only a single bead type is needed.

### EQUIVALENTS

The foregoing written specification is considered to be sufficient to enable one skilled in the art to practice the embodiments. The foregoing description and Examples detail certain embodiments and describes the best mode contemplated by the inventors. It will be appreciated, however, that no matter how detailed the foregoing may appear in text, the embodiment may be practiced in many ways and should be construed in accordance with the appended claims and any equivalents thereof.

As used herein, the term about refers to a numeric value, including, for example, whole numbers, fractions, and percentages, whether or not explicitly indicated. The term about generally refers to a range of numerical values (e.g., +/−5-10% of the recited range) that one of ordinary skill in the art would consider equivalent to the recited value (e.g., having the same function or result). When terms such as at least and about precede a list of numerical values or ranges, the terms modify all of the values or ranges provided in the list. In some instances, the term about may include numerical values that are rounded to the nearest significant figure.

1.-3. (canceled)

4. A method of normalizing extracted cfDNA comprising:
a. combining a solution comprising cfDNA with cellulose-coated beads, optionally wherein the cellulose-coated beads are prepared by a treatment with NaOH;
b. binding the cfDNA to the cellulose-coated beads in a binding buffer;
c. washing the bound cfDNA with a wash buffer; and
d. eluting the bound cfDNA with a resuspension buffer to prepare normalized cfDNA.

5. The method of claim 4, wherein the normalizing is performed with end-to-end automation.

6. The method of claim 5, further comprising preparing a nucleic acid library from the eluted cfDNA.

7. A method of normalizing a nucleic acid library comprising the steps of:
a. combining the library with cellulose-coated beads, optionally wherein the cellulose-coated beads are prepared by a treatment with NaOH;
b. binding the nucleic acid to the cellulose-coated beads in a binding buffer;
c. washing the bound nucleic acid with a wash buffer; and
d. eluting the bound nucleic acid with a resuspension buffer to prepare a normalized library.

8. The method of claim 7, wherein the method does not use carboxylate or streptavidin beads or uses only cellulose-coated beads.

9. (canceled)

10. The method of claim 7, wherein the normalizing is performed with treated cellulose-coated beads and the average fragment size comprised in the normalized library differs from the average fragment size comprised in the nucleic acid library before the normalizing by 15% or less, 10% or less, 5% or less, 4% or less, 3% or less, 2% or less, or 1% or less.

11. The method of claim 7, wherein the normalizing is performed with untreated cellulose-coated beads and the average fragment size comprised in the normalized library is 5% or more, 10% or more, 15% or more, 20% or more, 25% or more, or 30% or more larger than the average fragment size comprised in the nucleic acid library before the normalizing.

12. The method of claim 7, further comprising sequencing the normalized library.

13. The method of claim 12, wherein the relative sequencing coverage of fragments with a GC bias of 62% or more or with a GC bias of 60%-70% is higher for the normalized library as compared to the same nucleic acid library before the method of normalizing.

14. The method of claim 12, wherein the normalized library has 10% or more, 20% or more, 30% or more, 40% or more, 50% or more, 60% or more, 70% or more, or 80% or more greater sequencing coverage of fragments with a GC bias of 62% or more or with a GC bias of 60%-70% as compared to the same nucleic acid library before the method of normalizing.

15. The method of claim 7, wherein the library is a shotgun library.

16. The method of claim 7, wherein the nucleic acid library is prepared via the method of claim 6.

17. A kit for extracting cfDNA from a sample or normalizing a nucleic acid library comprising:
a. cellulose-coated beads, optionally wherein the cellulose-coated beads are prepared by treating with NaOH;
b. a binding buffer;
c. a wash buffer; and
d. a resuspension buffer.

18. The kit of claim 17, wherein the treatment with NaOH is treatment with 0.2 to 1.0M NaOH for 6 to 48 hours.

19. The kit of claim 17, wherein the binding buffer comprises PEG, Tris, GuSCN, and Tween-20.

20. The kit of claim 19, wherein the binding buffer comprises:
a. 30%-40% (weight/volume) of PEG200, PEG300, and/or PEG400;
b. 15-25 mM Tris pH 6.5 to pH 7;
c. 2-5M GuSCN; and
d. 0.1%-0.2% (weight/volume) Tween-20.

21. The kit of claim 17, wherein the wash buffer comprises PEG, Tris, GuSCN, and Tween-20.

22. The kit of claim 21, wherein the wash buffer comprises:
a. 15%-20% (weight/volume) PEG200, PEG300, and/or PEG400;
b. 8-12 mM Tris;
c. 1-3M GuSCN; and
d. 0.05%-0.1% (weight/volume) Tween-20.

23.-24. (canceled)

25. The kit of claim 17, wherein the cellulose-coated beads have a diameter of 1 μm to 10 μm.
